(19) United States
(12) Patent Application Publication
(10) Pub. No.: US 2008/0219978 A1
(43) Pub. Date: Sep. 11, 2008

(54) Soluble FcgammaRIA and related methods

(76) Inventor: Jeff L. Ellsworth, Seattle, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/011,190

(22) Filed: Jan. 23, 2008

Related U.S. Application Data
(60) Provisional application No. 60/886,211, filed on Jan. 23, 2007.
(60) Provisional application No. 60/886,367, filed on Jan. 24, 2007.

(51) Int. Cl. A61K 39/00

(52) U.S. Cl. 424/134.1

(57) ABSTRACT

Disclosed are soluble FcγRIA polypeptide compositions and related methods of using such polypeptides to treat IgG-mediated and immune complex-mediated inflammation. Also disclosed are related compositions and methods for producing the soluble FcγRIA polypeptides.

### CROSS-REFERENCE TO RELATED APPLICATIONS

The present application claims the benefit of U.S. Patent Application Ser. No. 60/886,211, filed Jan. 23, 2007 and U.S. Patent Application Ser. No. 60/886,367, filed Jan. 24, 2007, both of which are herein incorporated by reference in their entirety.

### BACKGROUND OF THE INVENTION

Immune system diseases are significant health-care problems that are growing at epidemic proportions. As such, they require novel, aggressive approaches to the development of new therapeutic agents. Standard therapy for autoimmune disease has been high dose, long-term systemic corticosteroids and immunosuppressive agents. The drugs used fall into three major categories: (1) glucocorticoids, such as prednisone and prednisolone; (2) calcineurin inhibitors, such as cyclosporine and tacrolimus; and (3) antiproliferative/antimetabolic agents such as azathioprine, sirolimus, and mycophenolate mofetil. Although these drugs have met with high clinical success in treating a number of autoimmune conditions, such therapies require lifelong use and act nonspecifically to suppress the entire immune system. The patients are thus exposed to significantly higher risks of infection and cancer. The calcineurin inhibitors and steroids are also nephrotoxic and diabetogenic, which has limited their clinical utility (Haynes and Fauci in Harrison's Principles of Internal Medicine, 16^{th} edition, Kasper et al., eds (2005), pp 1907-2066).

In addition to the conventional therapies for autoimmune disease, monoclonal antibodies and soluble receptors that target cytokines and their receptors have shown efficacy in a variety of autoimmune and inflammation diseases such as rheumatoid arthritis, organ transplantation, and Crohn's disease. Some of the agents include infliximab (REMICADE®) and etanercept (ENBREL®) that target tumor necrosis factor (TNF), muromonab-CD3 (ORTHOCLONE OKT3) that targets the T cell antigen CD3, and daclizumab (ZENAPAX®) that binds to CD25 on activated T cells, inhibiting signaling through this pathway. While efficacious in treating certain inflammatory conditions, use of these drugs has been limited by side effects including the “cytokine release syndrome” and an increased risk of infection (Krensky et al., in Goodman and Gilman's The Pharmacological Basis of Therapeutics, 10^{th} edition, Hardman and Limbird, eds, (2001), pp 1463-1484).

Passive immunization with intravenous immunoglobulin (IVIG) was licensed in the United States in 1981 for replacement therapy in patients with primary antibody deficiencies. Subsequent investigation showed that IVIG was also effective in ameliorating autoimmune symptoms in Kawasaki's disease and immune thrombocytopenia purpura (Lemieux et al., Mol. Immunol, 42:839-848, 2005; Ibanez and Montoro-Ronsano Curr. Pharm. Biotech., 4:239-247, 2003; Clynes, J. Clin. Invest., 115:25-27, 2005). IVIG has also been shown to reduce inflammation in adult dermatomyositis, Guillian-Barre syndrome, chronic inflammatory demyelinating polyneuropathies, multiple sclerosis, vasculitis, uveitis, myasthenia gravis, and in the Lambert-Eaton syndrome (Lemieux et al., supra; Ibanez and Montoro-Ronsano, supra).

IVIG is obtained from the plasma of large numbers (10,000-20,000) of healthy donors by cold ethanol fractionation. Commonly used IVIG preparations include Sandoglobulin, Flebogamma, Gammagard, Octagam, and Vigam S. In general, efficacy is seen when only large amounts of IVIG are infused into a patient, with an average dose of 2 g/kg/month used in autoimmune disease. The common (1-10% of patients) side effects of IVIG treatment include flushing, fever, myalgia, back pain, headache, nausea, vomiting, arthralgia, and dizziness. Uncommon (0.1-1% of patients) side effects include anaphylaxis, aseptic meningitis, acute renal failure, haemolytic anemia, and eczema. Although IVIG is generally considered safe, the pooled human plasma source is considered to be a risk factor for transfer of infectious agents. Thus, the use of IVIG is limited by its availability, high cost ($100/gm, including infusion cost), and the potential for severe adverse reactions (Lemieux et al., supra; Ibanez and Montoro-Ronsano, supra; Clynes, J. Clin. Invest., 115:25-27, 2005).

Numerous mechanisms have been proposed to explain the mode of action of IVIG, including regulation of Fc gamma receptor expression, increased clearance of pathogenic antibodies due to saturation of the neonatal Fc receptor FcRn, attenuation of complement-mediated damage, and modulation of T and B cells or the reticuloendothelial system (Clynes, supra). Since Fc domains purified from IVIG are as active as intact IgG in a number of in vitro and in vivo models of inflammation, it is well accepted that the anti-inflammatory properties of IVIG reside in the Fc domain of the IgG (Debre et al., Lancet, 342:945-949, 1993) or a sialylated subfraction (Kaneko et al., Science, 313:670-673, 2006).

Fc receptors for IgG (FcγR) play a unique role in mammalian biology by acting as a bridge between the innate and the acquired immune systems (Dijstelbloem et al., Trends Immunol. 22:510-516, 2001; Takai, Nature 2: 580-592, 2002; Nimmerjahn and Ravetch, Immunity 24: 19-28, 2006). By virtue of their binding to the Fc region of IgG (Woof and Burton, Nature Rev. Immunol., 4:1-11, 2004), FcγR regulate a variety of effector functions in ADCC, complement-mediated cell lysis, type III hypersensitivity reactions, tolerance, phagocytosis, antigen presentation, and the processing and clearance of immune complexes (Dijstelbloem et al., supra; Takai, supra; Nimmerjahn and Ravetch, supra).

The FcγR comprise three major gene families in humans including FcγRI (CD64), FcγRII (CD32), and FcγRIII (CD16) (Dijstelbloem et al., supra; Takai, supra). FcγRI is a high affinity receptor for monomeric IgG (10⁸-10⁹ M⁻¹) where FcγRII and FcγRIII exhibit low affinities for monomeric IgG (10⁷ M⁻¹) but bind to IgG immune complexes with greatly increased avidities. The FcγRII subfamily is composed of two major classes of genes, FcγRIIa and FcγRIIb, which after binding IgG transmit opposing signals to the cell interior. FcγRIIa contains an immunoreceptor tyrosine-activating motif (ITAM) within its short cytoplasmic tail, while FcγRIIb transmits inhibitory signals through an immunoreceptor tyrosine inhibitory motif (ITIM) within its cytoplasmic domain. FcγRIII subfamily also contains two distinct receptor genes, FcγRIIIa and FcγRIIIb. FcγRIIIa is a heterodimeric signaling receptor that after binding IgG immune complexes transmits activating signals through its associated ITAM-containing common γ chain. FcγRIIIb is bound to the cell membrane through a GPI linker and lacks intrinsic signaling capacity. FcγRI also lacks an intrinsic signaling capacity but similar to FcγRIIIa, associates with the common γ chain to transmit activating signals upon Fc binding. Signaling through FcγR involves kinase mediated phosphorylation/dephosphorylation events within the ITAM/ITIM sequences (Daeron, Intern. Rev. Immunol., 16: 1-27, 1997).

Consistent with their reported roles in immune biology, the human FcγR exhibit different affinities for subclasses of monomeric IgG: FcγRI binds IgG1≧IgG3>IgG4>>IgG2; FcγRIIa binds IgG3≧IgG1, IgG2>>IgG4; FcγRIIb binds IgG3≧IgG1>IgG4>IgG2; FcγRIIIa and FcγRIIIb bind IgG1, IgG3>>IgG2, IgG4 (Dijstelbloem et al., supra; Takai, supra).

In addition to differences in structure and signaling capacities, the FcγR also exhibit differences in cellular expression patterns. In humans, FcγRI is expressed predominantly on macrophages, monocytes, and neutrophils but can also be found on eosinophils and dendritic cells. FcγRIIa is the most widely expressed FcγR in humans and is expressed on platelets, macrophages, neutrophils, eosinophils, dendritic cells and Langerhans cells. FcγRIIb is the only FcγR expressed on B cells but is also expressed by mast cells, basophils, macrophages, eosinophils, neutrophils, dendritic and langerhan cells. FcγRIIIa is the only FcγR expressed on human NK cells and is widely expressed, found on macrophages, monocytes, mast cells, eosinophils, dendritic and langerhan cells. The expression of FcγRIIIb, on the other hand is largely restricted to neutrophils and eosinophils (Dijstelbloem et al., supra; Takai, supra).

Mice express FcγR that function similarly to the receptors in humans such as the orthologs of human high affinity FcγRI and the inhibitory receptor FcγRIIb (Nimmerjahn and Ravetch, Immunity, 24:19-28, 2006). The murine orthologs of human FcγRIIa and IIIa are thought to be FcγRIII and FcγRIV, respectively. Mice do not appear to express FcγRIIIb (Nimmerjahn and Ravetch, supra). Although some differences in cellular expression patterns have been noted, FcγR gene expression in humans and their orthologs in mice are generally similar.

Gene targeting in mice has suggested the importance of FcγR in the mammalian immune system (see generally Dijstelbloem et al., supra; Takai, supra; Nimmerjahn and Ravetch, supra). Deletion of the common γ chain, the signaling subunit of FcγRI, FcγRIII, and FcγRIV, abolishes signaling through all activating FcγR and renders mice resistant to a variety of autoimmune and inflammatory conditions. Mice deficient in the γ-chain exhibit attenuated immune complex-alveolitis, vasculitis, glomerulonephritis, Arthus reaction, and autoimmune hemolytic anemia. Similar data have been described for deletion of the α-chains of FcγRIII and FcγRI. FcγRIII −/− mice exhibit reduced immune complex-induced alveolitis, reduced sensitivity to autoimmune hemolytic anemia and an attenuated Arthus reaction. FcγRI −/− mice show impaired phagocytic function of macrophages, decreased cytokine release, attenuated ADCC and antigen presentation, reduced arthritis, enhanced antibody responses, and impaired hypersensitivity. Deletion of the inhibitory receptor, FcγRIIb, in contrast, results in augmented inflammation and autoimmune responses. FcγRIIb −/− mice show enhanced collagen-induced arthritis, spontaneous development of glomerulonephritis on a C57BL/6 background, enhanced Arthus reaction, enhanced alveolitis, enhanced IgG-induced systemic anaphylaxis, and enhanced anti-GBM induced glomerulonephritis. Thus, the FcγR play key roles in immune system homeostasis.

There is a need for Fc receptor antagonists, including FcγRI antagonists, useful in treating a variety of autoimmune diseases. Specifically, such antagonists would function to regulate the immune and hematopoietic systems, since disturbances of such regulation may be involved in disorders relating to inflammation, hemostasis, arthritis, immunodeficiency, and other immune and hematopoietic system anomalies. Therefore, there is a need for identification and characterization of such antagonists that can be used to prevent, ameliorate, or correct such disorders.

### BRIEF SUMMARY OF THE INVENTION

In one aspect, the present invention provides methods of reducing IgG-mediated inflammation in a subject. Such methods generally include administering to a subject with IgG-mediated inflammation an effective amount of a soluble FcγRIA polypeptide. In some embodiments of the method, the IgG-mediated inflammation is immune complex-mediated.

Typically, the soluble FcγRIA polypeptide comprises an amino acid sequence having at least 90% or at least 95% sequence identity with amino acid residues 16-282 of SEQ ID NO:2 and is capable of specifically binding the Fc region of IgG. For example, in some embodiments, the soluble FcγRIA polypeptide comprises amino acid residues 16-282 or 16-292 of SEQ ID NO:2. In other variations, the soluble FcγRIA polypeptide consists of amino acid residues 16-X of SEQ ID NO:2, where X is an integer from 282 to 292, inclusive.

In certain embodiments, the soluble FcγRIA polypeptide is a polypeptide produced by a recombinant production method in a host cell. Typically, such a production method includes culturing a cell into which has been introduced an expression vector having the following operably linked elements: (i) a transcription promoter; (ii) a DNA segment encoding a soluble polypeptide comprising an amino acid sequence having at least 90% or at least 95% sequence identity with amino acid residues 16-282 of SEQ ID NO:2, where the encoded polypeptide is capable of specifically binding the Fc region of IgG; and (iii) a transcription terminator. The cell is cultured under conditions whereby the cell expresses the polypeptide encoded by the DNA segment; and the expressed polypeptide is subsequently recovered. In some variations, the encoded polypeptide comprises amino acid residues 16-282 or 16-292 of SEQ ID NO:2. Particularly suitable polypeptides include, for example, polypeptides consisting of amino acid residues 16-X of SEQ ID NO:2, where X is an integer from 282 to 292, inclusive.

In other embodiments, the soluble FcγRIA polypeptide comprises an amino acid sequence having at least 90% or at least 95% sequence identity with amino acid residues 1-267 of SEQ ID NO:47, where the polypeptide is capable of specifically binding the Fc region of IgG. For example, in some embodiments, the soluble FcγRIA polypeptide comprises amino acid residues 1-267 or 1-277 of SEQ ID NO:47. In other variations, the soluble FcγRIA polypeptide consists of amino acid residues 1-X of SEQ ID NO:47, where X is an integer from 267 to 277, inclusive.

In certain aspects, methods of treating an IgG-mediated inflammatory disease are provided. Such methods generally include administering an effective amount of a soluble FcγRIA polypeptide as summarized above to a subject having the IgG-mediated inflammatory disease. The IgG-mediated inflammatory disease can be, for example, an autoimmune or immune complex-mediated disease. In particular variations, the IgG-mediated inflammatory disease is selected from the group consisting of rheumatoid arthritis (RA); systemic lupus erythematosus (SLE); mixed cryoglobulinemia; mixed connective tissue disease; a disease associated with an exonegous antigen; idiopathic thrombocytopenia purpura (ITP); Sjogren's syndrome; anti-phospholipid antibody syndrome; dermatomyositis; Guillain-Barre syndrome; and Goodpasture's syndrome.

Other aspects and variations of the present invention are further described herein.

### BRIEF DESCRIPTION OF THE DRAWINGS

FIG. 1 depicts blocking of immune complex precipitation in vitro with FcγRIA-CH₆. Anti-OVA/OVA immune complex precipitation assays were carried out as described in Example 15, infra. Each point represents the mean values of three separate experiments performed in duplicate. Circles: anti-OVA+OVA; triangles: anti-OVA+OVA+500 nM FcγRIA-CH6; squares: anti-OVA+OVA+1500 nM FcγRIA-CH6.

FIG. 2 depicts inhibition of immune complex-mediated production of inflammatory cytokines in mast cells with FcγRIA-CH6. Murine MC/9 mast cells were incubated with anti-OVA/OVA immune complexes in the presence of increasing amounts of FcγRIA-CH6 (“PFCGR1A CH6”) and secretion of inflammatory cytokines were determined as described in Example 15, infra. Each point represents the mean value of duplicate determinations and is representative of two separate experiments.

FIG. 3 depicts inhibition of immune complex-mediated edema and neutrophil infiltration in the murine Arthus reaction with FcγRIA-CH6. The cutaneous reversed passive Arthus reaction was established in mice using intradermal delivery of rabbit anti-ovalbumin and tail vein injection of ovalbumin. (See Example 15, infra.) Animals received either anti-OVA alone or anti-OVA together with the indicated amount of FcγRIA-CH6 (“pFCGR1A CH6”), and the effects of FcγRIA-CH6 on immune complex-mediated edema and neutrophil infiltration were assessed. (See id.) Each bar represents the mean ±SD for n=8 animals per group. 0.1x, 1.0x, and 7.0x pFCGRIA-CH6 represents the molar excess of FcγRIA-CH6 added relative to the amount of anti-OVA injected and is equivalent to 1.3 μg, 13.0 μg, and 91.0 μg of with FcγRIA-CH6, respectively.

FIG. 4 depicts inhibition of inflammation in the Arthus reaction in mice with systemic delivery of FcγRIA-CH6. Mice were injected with the indicated amounts of either vehicle alone or vehicle containing the indicated amount of FcγRIA-CH6 (“pFCGR1A CH6”) 1 hour prior to initiating the Arthus reaction. (See Example 15, infra.) Systemic administration of FcγRIA-CH6 was performed by intravenous injection, and the cutaneous reversed passive Arthus reaction was carried out using intradermal delivery of rabbit anti-ovalbumin, as described in Example 15. Edema was measured by anti-OVA induced extravasation of Evan's Blue dye. Each bar represents the mean ±SD for n=8 mice (intravenous injection of FcγRIA-CH6) or n=4 mice (intradermal injection of FcγRIA-CH6). The abbreviations used are: iv=intravenous; id=intradermal.

FIG. 5 depicts inhibition of edema in the Arthus reaction in mice with systemic delivery of FcγRIA-CH6. Mice were injected with the indicated amounts of either vehicle alone or vehicle containing the indicated amount of FcγRIA-CH6 (“pFCGR1A CH6”) 1 hour prior to initiating the Arthus reaction. (See Example 15, infra.) Systemic administration of FcγRIA-CH6 was performed by intravenous injection, and the cutaneous reversed passive Arthus reaction was carried out using intradermal delivery of rabbit anti-ovalbumin, as described in Example 15. Edema was measured by anti-OVA induced increases in tissue weights of the lesion sites. Each bar represents the mean ±SD for n=8 mice (intravenous injection of FcγRIA-CH6) or n=4 mice (intradermal injection of FcγRIA-CH6). The data are expressed relative to injection of nonimmune IgG. The abbreviations used are: iv=intravenous; id=intradermal.

FIGS. 6A-6D depicts FcγR1 sequences. FIG. 6A shows a polynucleotide sequence encoding FcγRIA (FcγR1 isoform a) (SEQ ID NO:1). FIG. 6B shows the polypeptide sequence of FcγRIA (SEQ ID NO:2). FIG. 6C shows the polypeptide sequence of the extracellular domain of FcγRIA (SEQ ID NO:3). FIG. 6D shows a comparison of FcγRIA polypeptide sequence with FcγR1 isoforms b1 (SEQ ID NO:4) and c (SEQ ID NO:5) polypeptide sequences. The vertical lines in FIG. 6D indicate where the introns are located in the corresponding gene; the triangle indicates the C-terminal amino acid of a particular embodiment of soluble FcγRIA or, alternatively, a C-terminal fusion site for certain tagged variations of soluble FcγRIA (e.g., His6-tagged FcγRIA). “16” above glutamine (Q) at amino acid position 16 in FIG. 6D indicates the amino terminal start site for the mature FcγRIA protein.

FIG. 7 depicts reduction of paw scores in the collagen antibody-induced arthritis mouse model with FcγRIA-CH6. Collagen antibody-induced arthritis was established in mice by treatment with the Arthrogen-CIA® antibody cocktail, as described in Example 17, infra. Mice also received either sub-cutaneous injections of either vehicle alone (PBS) or vehicle containing the indicated concentration of FcγRIA-CH6 (“PFCGR1A CH6”), every other day for a total of five doses. Each point represents the mean ±SEM for n=8 mice per group. Differences between groups were significant by repeated measures ANOVA.

FIG. 8 depicts reduction of paw thickness in the collagen antibody-induced arthritis mouse model with FcγRIA-CH6. Collagen antibody-induced arthritis was established in mice by treatment with the Arthrogen-CIA® antibody cocktail, as described in Example 17, infra. Mice also received either sub-cutaneous injections of either vehicle alone (PBS) or vehicle containing the indicated concentration of FcγRIA-CH6 (“PFCGR1A CH6”), every other day for a total of five doses. Each point represents the mean ±SEM for n=8 mice per group. Differences between groups were significant by repeated measures ANOVA.

FIGS. 9A-9C depict reduction in inflammation in the Arthus reaction by FcγRIA-CH6 but by neither FcγRIIA-CH6 nor FcγRIIIA-CH6. Experiments were carried out as described in Examples 15 and 16, infra. The data are expressed relative to that observed in the presence of anti-OVA alone after subtracting the values for non-immune IgG from each point. Each point, FcγRIA (), FcγRIIA (▴), FcγRIIIA (▪), represents the mean ±SEM for n=8-16 lesion sites (FIGS. 9A and 9B) and for n=5-13 lesion sites (FIG. 9C) from six separate experiments. Differences were significant, *p<0.0001 across all dose groups by ANOVA.

FIG. 10 depicts reduction in arthritis disease scores by treatment with FcγRIA. Collagen-induced arthritis (CIA) was established in mice as described in Example 19, infra. Once established disease was present, mice were treated with vehicle alone (PBS) (∘), or vehicle containing 0.22 mg or 2.0 mg FcγRIA (“FCGR1A”). (See Example 19, infra.) Each point represents the mean ±SE for 7-13 animals per group. Differences were significant, *p=0.001 by repeated measures ANOVA.

FIG. 11 depicts reduction in arthritis scores with an extended FcγRIA dose regimen. Collagen-induced arthritis (CIA) was established in mice as described in Example 19, infra. Mice were treated with vehicle alone (∘) or vehicle containing 2.0 mg FcγRIA dosed either every other day (▪) or every fourth day (▴). (See Example 19, infra.) Each point represents the mean ±SE for 7-13 animals per group. Differences were significant, *p=0.0125, **p=0.001 by repeated measures ANOVA. Evert fourth day dosing was for 11 days total.

FIG. 12 depicts reduction in the number of arthritic paws with FcγRIA treatment. Collagen-induced arthritis (CIA) was established in mice as described in Example 19, infra. Mice were treated every other day with vehicle alone (∘) or vehicle containing 0.22 mg FcγRIA (▴) or 2.0 mg (▪) of FcγRIA dosed either every other day. (See Example 19, infra.) Each point represents the mean of 7-13 mice per group.

### DESCRIPTION OF THE INVENTION

### I. Overview of the Invention

The present invention fills a need for novel therapeutics for treating IgG- and immune complex-mediated disease by providing Fc receptor antagonists, such as soluble FcγRIA. It was discovered that soluble FcγRIA, but not soluble FcγRIIA or FcγRIIIA, blocked inflammation in the cutaneous Arthus reaction (see Examples 15 and 16). Additionally, it was discovered that soluble FcγRIA also blocked the binding and signaling of immune complexes (described in detail in the Examples below) through cellular FcγR. The findings that soluble FcγRIA blocked inflammation in the cutaneous Arthus reaction, in the collagen antibody-induced model of arthritis, and in collagen-induced arthritis in mice were surprising, since FcγRIA, as a high affinity receptor for IgG Fc, is expected to be saturated with monomeric IgG in the circulation and hence generally less available for binding to immune complexes. These findings show that soluble FcγRIA is a potent therapeutic that can be used to treat autoimmune disease and inflammation.

Moreover, the soluble FcγRIA polypeptides described herein are useful to antagonize or block signaling of IgG and immune complexes in immune cells (e.g., lymphocytes, monocytes, leukocytes, macrohages and NK cells) for the treatment of IgG- and immune complex-mediated diseases such as, for example, autoimmune diabetes, multiple sclerosis (MS), systemic Lupus erythematosus (SLE), myasthenia gravis, Wegener's granulomatosis, Churg-Strauss syndrome, hepatitis-B-associated polyarteritis nodosa, microscopic polyangiitis, Henoch-Schonlein purpura, rheumatoid arthritis (RA), Lambert-Eaton syndrome, inflammatory bowel disease (IBD), essential mixed cryoglobulinemia, hepatitis-C-associated cryoglobulinemia, mixed connective tissue disease, autoimmune thrombocytopenias (ITP and TTP), adult dermatomyositis, Guillian-Barre syndrome, Sjogren's syndrome, Goodpasture's syndrome, chronic inflammatory demyelinating polyneuropathies, anti-phospholipid antibody syndrome, vasculitis, uveitis, serum sickness, pemphigus (e.g., pemphigus vulgaris), and diseases associated with exogenous antigens, such as viral and bacterial infections. Asthma, allergy, and other atopic disease may also be treated with the soluble FcγRIA polypeptides of the invention to inhibit the immune response or to deplete offending cells. Blocking or inhibiting signaling of IgG and immune complexes via Fcγ receptors, by using the soluble FcγRIA polypeptides of the present invention, may also benefit diseases of the pancreas, kidney, pituitary, and neuronal cells. The soluble FcγRIA polypeptides of the present invention are useful as antagonists of IgG and immune complexes. Such antagonistic effects can be achieved by direct neutralization or binding of the Fc domains IgG and immune complexes.

An illustrative nucleotide sequence that encodes human FcγRIA (isoform a of FcγRI) is provided by SEQ ID NO:1; the encoded polypeptide is shown in SEQ ID NO:2. FcγRIA is a receptor for the Fc domain of IgG. Analysis of a human cDNA clone encoding FcγRIA (SEQ ID NO:1) revealed an open reading frame encoding 374 amino acids (SEQ ID NO:2) comprising an extracellular ligand-binding domain of approximately 277 amino acid residues (residues 16-292 of SEQ ID NO:2; SEQ ID NO:3). Thus, in certain embodiments, polypeptides of the present invention include an IgG-binding domain comprising amino acids residues 16-292 of SEQ ID NO:2. In other variations, polypeptides of the present invention include an IgG-binding domain comprising amino acid residues 16-282 of SEQ ID NO:2

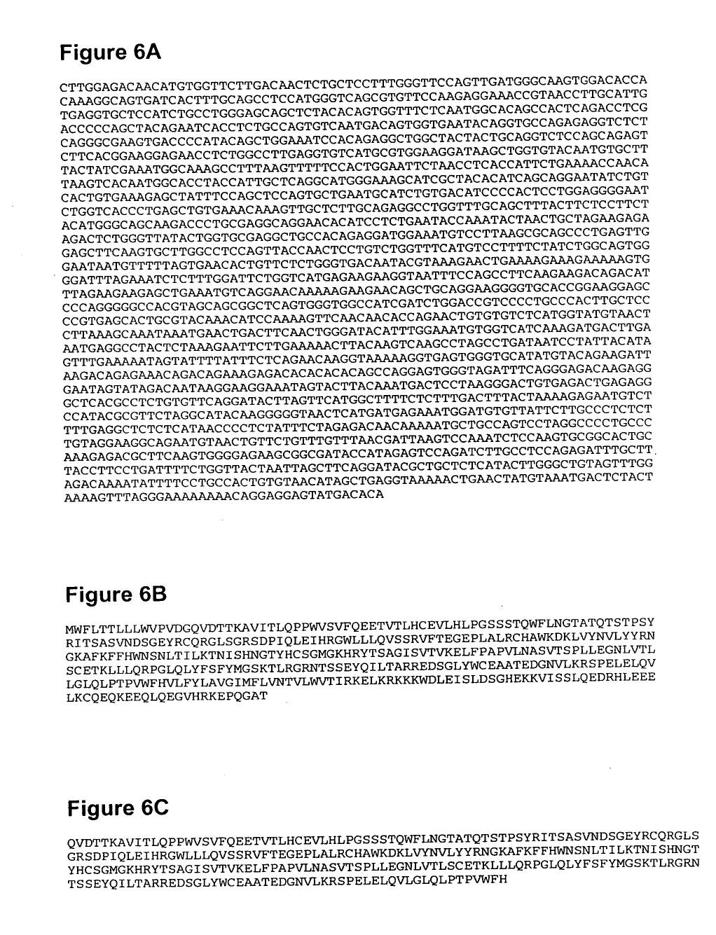

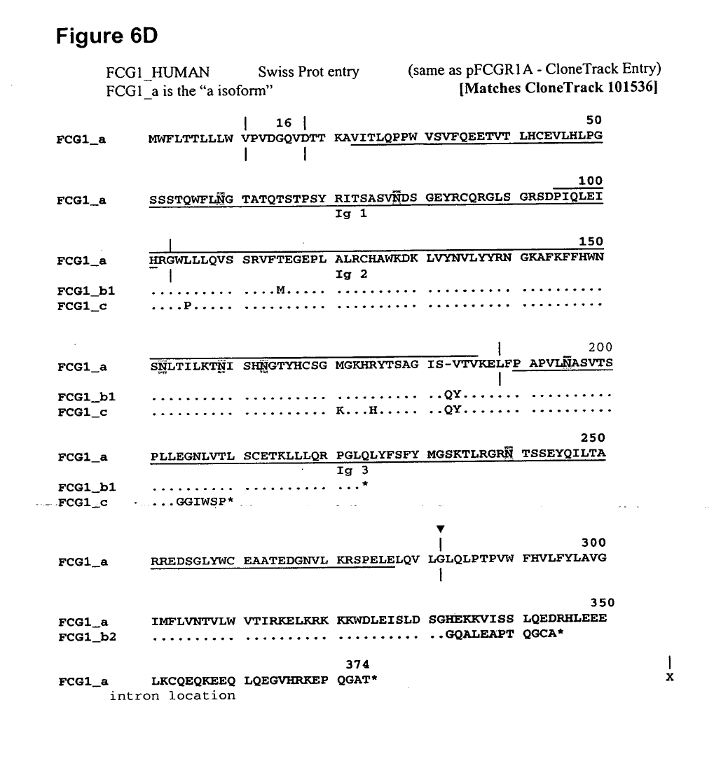

FcγRI also includes isoforms b1 and c, both of which are depicted in FIG. 6D as compared to FcγRIA (SEQ ID NO:2). Isoforms b1 and c comprise only two Ig domains, as opposed to isoform a, which comprises three Ig domains. Thus, a soluble FcγR1 polypeptide of the invention may comprise the extracellular domain of any of isoforms a, b1, or c as depicted in FIG. 6D. Additionally, if the soluble FcγR1 polypeptide is based on isoform b1 or c, the soluble FcγR1 polypeptide may also comprise the third Ig domain of isoform a.

Accordingly, in one aspect, the present invention provides isolated, soluble FcγRIA polypeptides capable of neutralizing IgG- or immune complex-mediated signaling in immune cells. Generally, a soluble FcγRIA polypeptide of the invention comprises an amino acid sequence that is at least 70%, at least 80%, at least 90%, at least 95%, at least 96%, at least 97%, at least 98%, or at least 99% or more identical to amino acid residues 16-282 or 16-292 of SEQ ID NO:2, wherein the isolated polypeptide is capable of specifically binding to the Fc domain of IgG (e.g., human IgG such as, for example, human IgG1). A soluble FcγRIA polypeptide of the invention specifically binds if it binds to monomeric human IgG (e.g., human IgG1) with a binding affinity (Kₐ) of at least 10⁶ M⁻¹, preferably at least 10⁷ M⁻¹, more preferably at least 10⁸ M⁻¹, and most preferably at least 10⁹ M⁻¹. In certain embodiments, a soluble FcγRIA polypeptide of the invention binds to monomeric human IgG with a binding affinity (Kₐ) of between 10⁸M⁻¹ and 10⁹ M⁻¹. The binding affinity of a soluble FcγRIA polypeptide can be readily determined by one of ordinary skill in the art, for example, by Scatchard analysis (Scatchard, Ann. NY Acad. Sci. 51:660, 1949). In addition to determining an affinity constant (Kₐ), an alternative means of measuring affinity is the equilibrium constant (K_{d}), where a decrease would be observed with the improvement in affinity. In certain embodiments, a soluble FcγRIA polypeptide of the invention binds to human IgG1 with an equilibrium dissociation constant (K_{d}) of less than 10⁻⁸ M, preferably less than 10⁻⁹ M, and more preferably less than 10⁻¹⁰ M. In a specific variation, a soluble FcγRIA polypeptide of the invention binds to human IgG1 with an equilibrium dissociation constant (K_{d}) of about 1.7×10⁻¹⁰ M. In some embodiments, a soluble FcγRIA polypeptide of the invention comprises amino acid residues 16-282 or 16-292 of SEQ ID NO:2. In specific variations, the soluble FcγRIA polypeptide consists of amino acid residues 16-X of SEQ ID NO:2, wherein X is an integer from 282 to 292, inclusive. Accordingly, in certain embodiments, a soluble FcγRIA polypeptide consists of amino acid residues 16-282, 16-283, 16-284, 16-285, 16-286, 16-287, 16-288, 16-289, 16-290, 16-291, or 16-292 of SEQ ID NO:2.

The present invention also provides isolated polypeptides and epitopes comprising at least 15 contiguous amino acid residues of an amino acid sequence of SEQ ID NO:3 (residues 16-292 of SEQ ID NO:2). Illustrative polypeptides include polypeptides that either comprise or consist of residues 16-282 or 16-292 of SEQ ID NO:2, or a functional IgG binding fragment thereof. Moreover, the present invention also provides isolated polypeptides as disclosed above that bind to, block, inhibit, reduce, antagonize or neutralize the activity of IgG, present in a monomeric form or as a multimeric immune complex.

The present invention also includes variant soluble FcγRIA receptor polypeptides, wherein the amino acid sequence of the variant soluble FcγRIA receptor polypeptide shares at least 70%, at least 80%, at least 90%, at least 95%, at least 96%, at least 97%, at least 98%, or at least 99% or more identity with the amino acid residues 16-282 or 16-292 of SEQ ID NO:2, and wherein any difference between the amino acid sequence of the variant polypeptide and the corresponding amino acid sequence of SEQ ID NO:2 is due to one or more conservative amino acid substitutions. Such conservative amino acid substitutions are described herein. Such variant soluble FcγRIA receptor polypeptides as provided by the present invention also bind to, block, inhibit, reduce, antagonize or neutralize the activity of IgG, present in a monomeric form or as a multimeric immune complex.

In another aspect, the present invention provides an isolated polynucleotide that encodes a soluble FcγRIA polypeptide as described herein. Generally, an isolated polynucleotide of the invention encodes a soluble FcγRIA polypeptide comprising an amino acid sequence that is at least 70%, at least 80%, at least 90%, at least 95%, at least 96%, at least 97%, at least 98%, or at least 99% or more identical to amino acid residues 16-282 or 16-292 of SEQ ID NO:2, wherein the encoded polypeptide is capable of specifically binding to the Fc domain of IgG (e.g., human IgG such as, for example, human IgG1). As described herein, the encoded soluble FcγRIA polypeptide is capable of neutralizing IgG- or immune complex-mediated signaling in immune cells. In specific variations, the encoded polypeptide comprises amino acid residues 16-282 or 16-292 of SEQ ID NO:2.

Within another aspect, the present invention provides an expression vector comprising the following operably linked elements: (a) a transcription promoter; a first DNA segment encoding a soluble FcγRIA polypeptide comprising an amino acid sequence that is at least 70%, at least 80%, at least 90%, at least 95%, at least 96%, at least 97%, at least 98%, or at least 99% or more identical to amino acid residues 16-282 or 16-292 of SEQ ID NO:2, wherein the encoded polypeptide is capable of specifically binding to the Fc domain of IgG (e.g., human IgG such as, for example, human IgG1); and a transcription terminator. In one embodiment, the expression vector disclosed above further comprises a secretory signal sequence operably linked to the first DNA segment (e.g., a DNA sequence encoding amino acid residues 1-15 of SEQ ID NO:2). In specific variations, the encoded polypeptide comprises amino acid residues 1-282, 16-282, 1-292, or 16-292 of SEQ ID NO:2.

Within another aspect, the present invention provides a cultured cell comprising an expression vector as disclosed above, wherein the cell expresses the soluble FcγRIA polypeptide encoded by the DNA segments. In another embodiment, the cultured cell is as disclosed above, wherein the cell secretes a soluble FcγRIA polypeptide. In another embodiment, the cultured cell is as disclosed above, wherein the cell secretes a soluble FcγRIA polypeptide that binds IgG or antagonizes IgG activity, where the IgG is present in a monomeric form or as a multimeric immune complex. In particular variations, the cultured cell is a mammalian cell such as, for example, a Chinese Hamster ovary (CHO) cell.

Within another aspect, the present invention provides an isolated soluble FcγRIA polypeptide comprising a sequence of amino acid residues that is at least 90% or at least 95% identical to amino acid residues 16-282 or 16-292 of SEQ ID NO:2, and wherein the soluble polypeptide binds IgG or antagonizes IgG activity, where the IgG is present in a monomeric form or as a multimeric immune complex.

Within another aspect, the present invention provides a method of producing a soluble FcγRIA polypeptide comprising culturing a cell as disclosed above; and isolating the soluble FcγRIA polypeptide produced by the cell.

Within another aspect, the present invention provides pharmaceutical compositions comprising a pharmaceutically acceptable carrier and a soluble FcγRIA polypeptide of the invention.

Within another aspect, the present invention also provides fusion proteins comprising an FcγRIA polypeptide and a heterologous polypeptide segment. Particularly suitable heterologous polypeptide segments include immunoglobulin moieties. In certain variations, the immunoglobulin moiety is an immunoglobulin heavy chain constant region, such as a human Fc fragment. The present invention further includes isolated nucleic acid molecules that encode such fusion proteins.

Within another aspect, the present invention provides a method for inhibiting IgG- or immune complex-induced proliferation of hematopoietic cells and hematopoietic cell progenitors comprising culturing bone marrow or peripheral blood cells with a composition comprising an amount of soluble FcγRIA sufficient to reduce proliferation of the hematopoietic cells in the bone marrow or peripheral blood cells as compared to bone marrow or peripheral blood cells cultured in the absence of soluble receptor. In one embodiment, the method is as disclosed above, wherein the hematopoietic cells and hematopoietic progenitor cells are lymphoid cells. In one embodiment, the method is as disclosed above, wherein the lymphoid cells are macrophages, B cells, or T cells. Within another aspect, the present invention provides a method for inhibiting antigen presentation by cells of the myeloid lineage such as macrophages or monocytes with a composition comprising an amount of soluble FcγRIA sufficient to reduce antigen presentation by myeloid-derived cells. In another embodiment, the method is as disclosed wherein the cells are B cells.

Within another aspect, the present invention provides a method of reducing IgG-mediated or immune-complex-mediated inflammation comprising administering to a mammal with inflammation an amount of a composition of a soluble FcγRIA sufficient to reduce inflammation.

Within another aspect, the present invention provides a method of suppressing an immune response in a mammal comprising administering a composition comprising a soluble FcγRIA polypeptide in an acceptable pharmaceutical vehicle.

Moreover, blocking the interaction between cell surface FcγR and the IgG Fc domains of immune complexes would attenuate the cellular response to the immune complexes and thus reduce inflammation. As such, the soluble FcγRIA polypeptides of the present invention, which as shown herein are effective in blocking IgG- and immune complex-mediated immune responses, are useful in therapeutic treatment of inflammatory diseases such as, for example, arthritis (e.g., rheumatoid arthritis or psoriatic arthritis), adult respiratory disease (ARD), endotoxemia, septic shock, multiple organ failure, inflammatory lung injury (e.g., asthma or bronchitis), bacterial pneumonia, psoriasis, eczema, atopic and contact dermatitis, inflammatory bowel disease (IBD) (e.g., ulcerative colitis or Crohn's disease), and aberrant immune responses to bacterial or viral infection.

These and other aspects of the invention will become evident upon reference to the following detailed description. In addition, various references are identified herein are incorporated by reference in their entirety.

### II. Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “complement of a nucleic acid molecule” refers to a nucleic acid molecule having a complementary nucleotide sequence and reverse orientation as compared to a reference nucleotide sequence.

The term “degenerate nucleotide sequence” denotes a sequence of nucleotides that includes one or more degenerate codons as compared to a reference nucleic acid molecule that encodes a polypeptide. Degenerate codons contain different triplets of nucleotides, but encode the same amino acid residue (i.e., GAU and GAC triplets each encode Asp).

The term “structural gene” refers to a nucleic acid molecule that is transcribed into messenger RNA (mRNA), which is then translated into a sequence of amino acids characteristic of a specific polypeptide.

An “isolated nucleic acid molecule” is a nucleic acid molecule that is not integrated in the genomic DNA of an organism. For example, a DNA molecule that encodes a growth factor that has been separated from the genomic DNA of a cell is an isolated DNA molecule. Another example of an isolated nucleic acid molecule is a chemically-synthesized nucleic acid molecule that is not integrated in the genome of an organism. A nucleic acid molecule that has been isolated from a particular species is smaller than the complete DNA molecule of a chromosome from that species.

A “nucleic acid molecule construct” is a nucleic acid molecule, either single- or double-stranded, that has been modified through human intervention to contain segments of nucleic acid combined and juxtaposed in an arrangement not existing in nature.

“Linear DNA” denotes non-circular DNA molecules having free 5′ and 3′ ends. Linear DNA can be prepared from closed circular DNA molecules, such as plasmids, by enzymatic digestion or physical disruption.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

A “promoter” is a nucleotide sequence that directs the transcription of a structural gene. Typically, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). If a promoter is an inducible promoter, then the rate of transcription increases in response to an inducing agent. In contrast, the rate of transcription is not regulated by an inducing agent if the promoter is a constitutive promoter. Repressible promoters are also known.

A “core promoter” contains essential nucleotide sequences for promoter function, including the TATA box and start of transcription. By this definition, a core promoter may or may not have detectable activity in the absence of specific sequences that may enhance the activity or confer tissue specific activity.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner.

An “enhancer” is a type of regulatory element that can increase the efficiency of transcription, regardless of the distance or orientation of the enhancer relative to the start site of transcription.

“Heterologous DNA” refers to a DNA molecule, or a population of DNA molecules, that does not exist naturally within a given host cell. DNA molecules heterologous to a particular host cell may contain DNA derived from the host cell species (i.e., endogenous DNA) so long as that host DNA is combined with non-host DNA (i.e., exogenous DNA). For example, a DNA molecule containing a non-host DNA segment encoding a polypeptide operably linked to a host DNA segment comprising a transcription promoter is considered to be a heterologous DNA molecule. Conversely, a heterologous DNA molecule can comprise an endogenous gene operably linked with an exogenous promoter. As another illustration, a DNA molecule comprising a gene derived from a wild-type cell is considered to be heterologous DNA if that DNA molecule is introduced into a mutant cell that lacks the wild-type gene.

A “polypeptide” is a polymer of amino acid residues joined by peptide bonds, whether produced naturally or synthetically. Polypeptides of less than about 10 amino acid residues are commonly referred to as “peptides.”

In the polypeptide context, the term “fragment” refers to a portion of a polypeptide typically having at least 20 contiguous or at least 50 contiguous amino acids of the polypeptide. A “variant” includes a polypeptide or fragment thereof having amino acid substitutions (e.g., conservative amino acid substitutions) relative to a second polypeptide; or a polypeptide or fragment thereof that is modified by covalent attachment of a second molecule such as, e.g., by attachment of a heterologous polypeptide, or by glycosylation, acetylation, phosphorylation, and the like. Further included within the definition of “polypeptide” is, for example, polypeptides containing one or more analogs of an amino acid (e.g., unnatural amino acids and the like), polypeptides with unsubstituted linkages, as well as other modifications known in the art, both naturally and non-naturally occurring.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

A peptide or polypeptide encoded by a non-host DNA molecule is a “heterologous” peptide or polypeptide.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter.

A “recombinant host” is a cell that contains a heterologous nucleic acid molecule, such as a cloning vector or expression vector. In the present context, an example of a recombinant host is a cell that produces FcγRIA from an expression vector. In contrast, FcγRIA can be produced by a cell that is a “natural source” of FcγRIA, and that lacks an expression vector.

“Integrative transformants” are recombinant host cells, in which heterologous DNA has become integrated into the genomic DNA of the cells.

A “fusion protein” is a hybrid protein expressed by a nucleic acid molecule comprising nucleotide sequences of at least two genes. For example, a fusion protein can comprise at least part of an FcγRIA polypeptide fused with a polypeptide that binds an affinity matrix. Such a fusion protein provides a means to isolate large quantities of FcγRIA using affinity chromatography.

The term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

In general, the binding of ligand to receptor results in a conformational change in the receptor that causes an interaction between the effector domain and other molecule(s) in the cell, which in turn leads to an alteration in the metabolism of the cell. Metabolic events that are often linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids.

A “soluble receptor” is a receptor polypeptide that is not bound to a cell membrane. Soluble receptors are most commonly ligand-binding receptor polypeptides that lack transmembrane and cytoplasmic domains, and other linkage to the cell membrane such as via glycophosphoinositol (gpi). Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Soluble receptors can be monomeric, homodimeric, heterodimeric, or multimeric, with multimeric receptors generally not comprising more than 9 subunits, preferably not comprising more than 6 subunits, and most preferably not comprising more than 3 subunits. Receptor polypeptides are said to be substantially free of transmembrane and intracellular polypeptide segments when they lack sufficient portions of these segments to provide membrane anchoring or signal transduction, respectively. Moreover, one of skill in the art using the genetic code can readily determine polynucleotides that encode such soluble receptor polypeptides.

The term “secretory signal sequence” denotes a DNA sequence that encodes a peptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, such as 96%, 97%, or 98% or more pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “expression” refers to the biosynthesis of a gene product. For example, in the case of a structural gene, expression involves transcription of the structural gene into mRNA and the translation of mRNA into one or more polypeptides.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

As used herein, the term “immunomodulator” includes cytokines, stem cell growth factors, lymphotoxins, co-stimulatory molecules, hematopoietic factors and the like, and synthetic analogs of these molecules.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of less than 10⁹ M⁻¹.

An “antibody fragment” is a portion of an antibody such as F(ab′)₂, F(ab)₂, Fab′, Fab, and the like. Regardless of structure, an antibody fragment binds with the same antigen that is recognized by the intact antibody. For example, an anti-FcγRIA monoclonal antibody fragment binds with an epitope of FcγRIA.

The term “antibody fragment” also includes a synthetic or a genetically engineered polypeptide that binds to a specific antigen, such as polypeptides consisting of the light chain variable region, “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

A “chimeric antibody” is a recombinant protein that contains the variable domains and complementary determining regions derived from a rodent antibody, while the remainder of the antibody molecule is derived from a human antibody.

“Humanized antibodies” are recombinant proteins in which murine complementarity determining regions of a monoclonal antibody have been transferred from heavy and light variable chains of the murine immunoglobulin into a human variable domain. Construction of humanized antibodies for therapeutic use in humans that are derived from murine antibodies, such as those that bind to or neutralize a human protein, is within the skill of one in the art.

As used herein, a “therapeutic agent” is a molecule or atom which is conjugated to an antibody moiety to produce a conjugate which is useful for therapy. Examples of therapeutic agents include drugs, toxins, immunomodulators, chelators, boron compounds, photoactive agents or dyes, and radioisotopes.

A “detectable label” is a molecule or atom which can be conjugated to an antibody moiety to produce a molecule useful for diagnosis. Examples of detectable labels include chelators, photoactive agents, radioisotopes, fluorescent agents, paramagnetic ions, or other marker moieties.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enzymol 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNA molecules encoding affinity tags are available from commercial suppliers (e.g., Pharmacia Biotech, Piscataway, N.J.).

A “naked antibody” is an entire antibody, as opposed to an antibody fragment, which is not conjugated with a therapeutic agent. Naked antibodies include both polyclonal and monoclonal antibodies, as well as certain recombinant antibodies, such as chimeric and humanized antibodies.

As used herein, the term “antibody component” includes both an entire antibody and an antibody fragment.

An “immunoconjugate” is a conjugate of an antibody component with a therapeutic agent or a detectable label.

As used herein, the term “antibody fusion protein” refers to a recombinant molecule that comprises an antibody component and an FcγRIA polypeptide component. Examples of an antibody fusion protein include a protein that comprises an FcγRIA extracellular domain, and either an Fc domain or an antigen-binding region.

A “target polypeptide” or a “target peptide” is an amino acid sequence that comprises at least one epitope, and that is expressed on a target cell, such as a tumor cell, or a cell that carries an infectious agent antigen. T cells recognize peptide epitopes presented by a major histocompatibility complex molecule to a target polypeptide or target peptide and typically lyse the target cell or recruit other immune cells to the site of the target cell, thereby killing the target cell.

An “antigenic peptide” is a peptide which will bind a major histocompatibility complex molecule to form an MHC-peptide complex which is recognized by a T cell, thereby inducing a cytotoxic lymphocyte response upon presentation to the T cell. Thus, antigenic peptides are capable of binding to an appropriate major histocompatibility complex molecule and inducing a cytotoxic T cells response, such as cell lysis or specific cytokine release against the target cell which binds or expresses the antigen. The antigenic peptide can be bound in the context of a class I or class II major histocompatibility complex molecule, on an antigen presenting cell or on a target cell.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

An “anti-sense oligonucleotide specific for FcγRIA” or a “FcγRIA anti-sense oligonucleotide” is an oligonucleotide having a sequence (a) capable of forming a stable triplex with a portion of the FcγRIA gene, or (b) capable of forming a stable duplex with a portion of an mRNA transcript of the FcγRIA gene.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

The term “variant FcγRIA receptor gene” or “variant FcγRIA polynucleotide” refers to nucleic acid molecules that encode a polypeptide having an amino acid sequence that is a modification of SEQ ID NO:2. Such variants include naturally-occurring polymorphisms of FcγRIA receptor genes, as well as synthetic genes that contain conservative amino acid substitutions of the amino acid sequence of SEQ ID NO:2. Additional variant forms of FcγRIA receptor genes are nucleic acid molecules that contain insertions or deletions of the nucleotide sequences described herein. A variant FcγRIA receptor gene can be identified, for example, by determining whether the gene hybridizes with a nucleic acid molecule having the nucleotide sequence of SEQ ID NO:1, or its complement, under stringent conditions.

Alternatively, variant FcγRIA receptor genes can be identified by sequence comparison. Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art (see, for example, Peruski and Peruski, The Internet and the New Biology: Tools for Genomic and Molecular Research (ASM Press, Inc. 1997), Wu et al. (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2nd Edition (Academic Press, Inc. 1998)). Particular methods for determining sequence identity are described below.

In certain variations, a variant FcγRIA polypeptide comprises the third Ig domain of FcγRIA fused to the first and second Ig domains of another Fcγ receptor, such as the first and second Ig domains of FcγRI isoform b1 or of FcγRI isoform c.

Regardless of the particular method used to identify a variant FcγRIA receptor gene or variant FcγRIA polypeptide, a variant gene or polypeptide encoded by a variant gene may be functionally characterized by the ability to bind to IgG, using a biological or biochemical assay described herein.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “ortholog” denotes a polypeptide or protein obtained from one species that is the functional counterpart of a polypeptide or protein from a different species. Sequence differences among orthologs are the result of speciation.

“Paralogs” are distinct but structurally related proteins made by an organism. Paralogs are believed to arise through gene duplication. For example, α-globin, β-globin, and myoglobin are paralogs of each other.

The present invention includes functional fragments of FcγRIA receptor genes. Within the context of this invention, a “functional fragment” of a FcγRIA receptor gene refers to a nucleic acid molecule that encodes a portion of a FcγRIA polypeptide which is a domain described herein or at least binds to IgG.

“Corresponding to”, when used in reference to a nucleotide or amino acid sequence, indicates the position in a second sequence that aligns with the reference position when two sequences are optimally aligned.

With regard to FcγR polypeptides as described herein (e.g., soluble FcγRIA polypeptides comprising residues 16-282 or 16-292 of SEQ ID NO:2), reference to amino acid residues corresponding to those specified by SEQ ID NO includes post-translational modifications of such residues. For example, reference to a glutamine at a position corresponding to position 16 of SEQ ID NO:2 encompasses a post-translational modification of this glutamine to pyro-glutamic acid.

“Immune complex,” as used herein, refers to a complex that forms upon binding of an IgG antibody to its cognate antigen. The term “immune complex” as used herein encompasses all stoichiometries of antigen:antibody complexes. For example, an immune complex may comprise a single IgG antibody (monomeric IgG) bound to antigen or may comprise multiple IgG antibodies bound to antigen (multimeric immune complex).

“IgG-mediated inflammation,” as used herein, refers to an inflammatory response mediated at least in part by the binding of an immune complex to an Fcγ receptor via the Fc region of an IgG antibody contained within the immune complex. “IgG-mediated inflammation” also encompasses the activation of the complement pathway by IgG immune complexes.

“Immune complex-mediated inflammation,” as used herein, refers to IgG-mediated inflammation characterized at least in part by the deposition of immune complexes within one or more tissues.

“IgG-mediated disease” or “IgG-mediated inflammatory disease,” as used herein, refers to an inflammatory disease mediated at least in part by the binding of an immune complex to an Fcγ receptor via the Fc region of an IgG antibody contained within the immune complex. “IgG-mediated disease” or “IgG-mediated inflammatory disease” also encompasses diseases characterized at least in part by the activation of the complement pathway by IgG immune complexes.

“Autoimmune disease,” as used herein, refers to an IgG-mediated inflammatory disease characterized at least in part by the presence of IgG autoantibodies, i.e., IgG antibodies specific for one or more self-antigens. Autoimmune diseases include, for example, diseases associated with autoantibody production as well as the deposition of immune complexes in one or more tissues; such diseases include, e.g., systemic lupus erythematosus (SLE), rheumatoid arthritis (RA), and mixed connective tissue disease. Autoimmune diseases also include those diseases associated with autoantibody production although not clearly associated with deposition of immune complexes, such as, for example, idiopathic thrombocytopenia purpura (ITP), Sjogren's Syndrome, antiphospholipid antibody syndrome, dermatomyositis, Guillain-Barre Syndrome, and Goodpasture's Syndrome. Other autoimmune diseases include, e.g., inflammatory bowel disease (IBD), psoriasis, atopic dermatitis, myasthenia gravis, type I diabetes, and multiple sclerosis.

“Immune complex-mediated disease,” as used herein, refers to an IgG-mediated inflammatory disease characterized at least in part by the deposition of immune complexes within one or more tissues. Immune complex-mediated diseases include, for example, mixed cryoglobulinemia; systemic lupus erythematosus (SLE); rheumatoid arthritis (RA); mixed connective tissue disease; and diseases associated with exonegous antigens such as, e.g., HBV-associated polyarteritis nodosa.

Due to the imprecision of standard analytical methods, molecular weights and lengths of polymers are understood to be approximate values. When such a value is expressed as “about” X or “approximately” X, the stated value of X will be understood to be accurate to ±10%.

### III. Production of FcγRIA Polynucleotides or Genes

Polynucleotides encoding a human FcγRIA receptor gene can be obtained by screening a human cDNA or genomic library using polynucleotide probes based upon SEQ ID NO:1. These techniques are standard and well-established, and may be accomplished using cloning kits available by commercial suppliers. See, for example, Ausubel et al. (eds.), Short Protocols in Molecular Biology, 3^{rd} Edition, John Wiley & Sons 1995; Wu et al., Methods in Gene Biotechnology, CRC Press, Inc. 1997; Aviv and Leder, Proc. Nat'l Acad. Sci. USA 69:1408 (1972); Huynh et al., “Constructing and Screening cDNA Libraries in λgt10 and λgt11,” in DNA Cloning: A Practical Approach Vol. I, Glover (ed.), page 49 (IRL Press, 1985); Wu (1997) at pages 47-52.

Polynucleotides that encode a human FcγRIA receptor gene can also be obtained using the polymerase chain reaction (PCR) with oligonucleotide primers having nucleotide sequences that are based upon the nucleotide sequences of the FcγRIA receptor gene or cDNA. General methods for screening libraries with PCR are provided by, for example, Yu et al., “Use of the Polymerase Chain Reaction to Screen Phage Libraries,” in Methods in Molecular Biology, Vol. 15. PCR Protocols: Current Methods and Applications, White (ed.), Humana Press, Inc., 1993. Moreover, techniques for using PCR to isolate related genes are described by, for example, Preston, “Use of Degenerate Oligonucleotide Primers and the Polymerase Chain Reaction to Clone Gene Family Members,” in Methods in Molecular Biology, Vol. 15: PCR Protocols: Current Methods and Applications, White (ed.), Humana Press, Inc. 1993. As an alternative, an FcγRIA gene can be obtained by synthesizing nucleic acid molecules using mutually priming long oligonucleotides and the nucleotide sequences described herein (see, e.g., Ausubel (1995)). Established techniques using the polymerase chain reaction provide the ability to synthesize DNA molecules at least two kilobases in length (Adang et al., Plant Molec. Biol. 21:1131, 1993, Bambot et al., PCR Methods and Applications 2:266, 1993, Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993), and Holowachuk et al., PCR Methods Appl. 4:299, 1995). For reviews on polynucleotide synthesis, see, for example, Glick and Pasternak, Molecular Biotechnology, Principles and Applications of Recombinant DNA (ASM Press 1994), Itakura et al., Annu. Rev. Biochem. 53:323 (1984), and Climie et al., Proc. Nat'l Acad. Sci. USA 87:633, 1990.

### IV. Production of FcγRIA Gene Variants

The present invention provides a variety of nucleic acid molecules, including DNA and RNA molecules, that encode the FcγRIA polypeptides disclosed herein. Those skilled in the art will readily recognize that, in view of the degeneracy of the genetic code, considerable sequence variation is possible among these polynucleotide molecules. Moreover, the present invention also provides isolated soluble monomeric and homodimeric polypeptides that comprise at least one FcγRIA polypeptide subunit that is substantially homologous to the polypeptide of residues 16-282 or 16-292 of SEQ ID NO:2. Thus, the present invention contemplates FcγRIA polypeptide-encoding nucleic acid molecules comprising degenerate nucleotides of SEQ ID NO:1, and their RNA equivalents.

Table 1 sets forth the one-letter codes to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding an amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

Different species can exhibit “preferential codon usage.” In general, see, Grantham et al., Nucl. Acids Res. 8:1893 (1980), Haas et al. Curr. Biol. 6:315 (1996), Wain-Hobson et al., Gene 13:355 (1981), Grosjean and Fiers, Gene 18:199 (1982), Holm, Nuc. Acids Res. 14:3075 (1986), Ikemura, J. Mol. Biol. 158:573 (1982), Sharp and Matassi, Curr. Opin. Genet. Dev. 4:851 (1994), Kane, Curr. Opin. Biotechnol. 6:494 (1995), and Makrides, Microbiol. Rev. 60:512 (1996). As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequences disclosed herein serve as a template for optimizing expression of polynucleotides in various cell types and species commonly used in the art and disclosed herein. Sequences containing preferential codons can be tested and optimized for expression in various species, and tested for functionality as disclosed herein.

An FcγRIA-encoding cDNA can be isolated by a variety of methods, such as by probing with a complete or partial human cDNA or with one or more sets of degenerate probes based on the disclosed sequences. A cDNA can also be cloned using the polymerase chain reaction with primers designed from the representative human FcγRIA sequences disclosed herein. In addition, a cDNA library can be used to transform or transfect host cells, and expression of the cDNA of interest can be detected with an antibody to an FcγRIA polypeptide.

Those skilled in the art will recognize that the sequence disclosed in SEQ ID NO:1 represents a single allele of human FcγRIA, and that allelic variation and alternative splicing are expected to occur. Allelic variants of this sequence can be cloned by probing cDNA or genomic libraries from different individuals according to standard procedures. Allelic variants of the nucleotide sequences disclosed herein, including those containing silent mutations and those in which mutations result in amino acid sequence changes, are within the scope of the present invention, as are proteins which are allelic variants of the amino acid sequences disclosed herein. cDNA molecules generated from alternatively spliced mRNAs, which retain the properties of the FcγRIA polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Using the methods discussed above, one of ordinary skill in the art can prepare a variety of polypeptides that comprise a soluble FcγRIA polypeptide that is substantially homologous to residues 16-282 or 16-292 of SEQ ID NO:2 and that retain the ligand-binding properties of the wild-type FcγRIA, including allelic variants thereof, as well as polynucleotides encoding such variants. Such polypeptides may also include additional polypeptide segments as generally disclosed herein.

Within certain embodiments of the invention, the isolated nucleic acid molecules can hybridize under stringent conditions to nucleic acid molecules comprising nucleotide sequences disclosed herein. For example, such nucleic acid molecules can hybridize under stringent conditions to nucleic acid molecules comprising the nucleotide sequence of SEQ ID NO:1, or to nucleic acid molecules comprising a nucleotide sequence complementary to SEQ ID NO:1, or fragments thereof.

In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is at least about 0.02 M at pH 7 and the temperature is at least about 60° C. As previously noted, the isolated polynucleotides of the present invention include DNA and RNA. Methods for isolating DNA and RNA are well known in the art. It is generally preferred to isolate RNA from pancreas or prostate tissues although cDNA can also be prepared using RNA from other tissues or isolated as genomic DNA. Total RNA can be prepared using guanidine HCl extraction followed by isolation by centrifugation in a CsCl gradient (Chirgwin et al., Biochemistry 18:52-94, (1979)). Poly (A)⁺ RNA is prepared from total RNA using the method of Aviv and Leder Proc. Natl. Acad. Sci. USA 69:1408-1412, (1972). Complementary DNA (cDNA) is prepared from poly(A)⁺ RNA using known methods. Polynucleotides encoding FcγRIA polypeptides are then identified and isolated by, for example, hybridization or PCR.

Those skilled in the art will recognize that the sequences disclosed in SEQ ID NO:2 and the corresponding nucleotides of SEQ ID NO:1 and represent single alleles of the human FcγRIA receptor. Allelic variants of these sequences can be cloned by probing cDNA or genomic libraries from different individuals according to standard procedures.

The present invention further provides counterpart receptors and polynucleotides from other species (“species orthologs”). Of particular interest are FcγRIA polypeptides from other mammalian species, including murine, porcine, ovine, bovine, canine, feline, equine, and non-human primates. Species orthologs of the human FcγRIA receptor can be cloned using information and compositions provided by the present invention in combination with conventional cloning techniques. For example, a cDNA can be cloned using mRNA obtained from a tissue or cell type that expresses the receptor. Suitable sources of mRNA can be identified by probing Northern blots with probes designed from the sequences disclosed herein. A library is then prepared from mRNA of a positive tissue or cell line. A receptor-encoding cDNA can then be isolated by a variety of methods, such as by probing with a complete or partial cDNA of human and other primates or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to the receptor. Similar techniques can also be applied to the isolation of genomic clones.

The present invention also provides isolated soluble monomeric, homodimeric, heterodimeric and multimeric FcγRIA polypeptides that comprise at least one FcγRIA receptor subunit that is substantially homologous to the polypeptide of residues 16-282 or 16-292 of SEQ ID NO:2. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. The term “substantially homologous” is used herein to denote polypeptides having 50%, preferably 60%, more preferably at least 80%, sequence identity to the sequences shown in SEQ ID NO:2. Such polypeptides will more preferably be at least 90% identical, and most preferably 95% or more identical to SEQ ID NO:3. Percent sequence identity is determined by conventional methods. (See, e.g., Altschul et al., Bull. Math. Bio. 48: 603-616, (1986) and Henikoff and Henikoff, Proc. Natl. Acad. Sci. USA 89:10915-10919, 1992.) Briefly, two amino acid sequences are aligned to optimize the alignment scores using a gap opening penalty of 10, a gap extension penalty of 1, and the “blossom 62” scoring matrix of Henikoff and Henikoff (id.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant ztryp1. The FASTA algorithm is described by Pearson and Lipman, Proc. Nat'l Acad. Sci. USA 85:2444, 1988, and by Pearson, Meth. Enzymol. 183:63, 1990.

Briefly, FASTA first characterizes sequence similarity by identifying regions shared by the query sequence (e.g., residues 16-282 or 16-292 of SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol., supra.

FASTA can also be used to determine the sequence identity of nucleic acid molecules using a ratio as disclosed above. For nucleotide sequence comparisons, the ktup value can range between one to six, preferably from three to six, most preferably three, with other FASTA program parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915, 1992). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Substantially homologous proteins and polypeptides are characterized as having one or more amino acid substitutions, deletions or additions. These changes are preferably of a minor nature, that is conservative amino acid substitutions (see Table 4) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), or other antigenic epitope or binding domain. (See generally Ford et al., Protein Expression and Purification 2:95-107, 1991.) DNAs encoding affinity tags are available from commercial suppliers (e.g., Pharmacia Biotech, Piscataway, N.J.).

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Essential amino acids in the receptor polypeptides of the present invention can be identified according to procedures known in the art, such as site-directed mutagenesis or alanine-scanning mutagenesis (Cunningham and Wells, Science 244:1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand-receptor interaction can also be determined by analysis of crystal structure as determined by such techniques as nuclear magnetic resonance, crystallography or photoaffinity labeling. (See, e.g., de Vos et al., Science 255:306-312, 1992; Smith et al., J. Mol. Biol. 224:899-904, 1992; Wlodaver et al., FEBS Lett. 309:59-64, 1992.) The identities of essential amino acids can also be inferred from analysis of homologies with related receptors.

Multiple amino acid substitutions can be made and tested using known methods of mutagenesis and screening, such as those disclosed by Reidhaar-Olson and Sauer Science 241:53-57, 1988 or Bowie and Sauer Proc. Natl. Acad. Sci. USA 86:2152-2156, 1989. Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display e.g., Lowman et al., Biochem. 30:10832-10837, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Mutagenesis methods as disclosed above can be combined with high-throughput screening methods to detect activity of cloned, mutagenized receptors in host cells. Preferred assays in this regard include cell proliferation assays and biosensor-based ligand-binding assays, which are described below. Mutagenized DNA molecules that encode active receptors or portions thereof (e.g., ligand-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptides that comprise a soluble FcγRIA polypeptide that is substantially homologous to residues 16-282 or 16-292 of SEQ ID NO:2 or allelic variants thereof and retain the ligand-binding properties (i.e. IgG binding properties) of the wild-type receptor. Such polypeptides may include additional amino acids from an extracellular ligand-binding domain of a FcγRIA receptor as well as part or all of the transmembrane and intracellular domains. Such polypeptides may also include additional polypeptide segments as generally disclosed above.

### V. Production of FcγRIA Polypeptides

The polypeptides of the present invention can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., (1989), and Ausubel et al., ibid., which are incorporated herein by reference.

In general, a DNA sequence encoding a soluble FcγRIA polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers. Multiple components of a soluble receptor complex can be co-transfected on individual expression vectors or be contained in a single expression vector. Such techniques of expressing multiple components of protein complexes are well known in the art.

To direct a soluble FcγRIA polypeptide into the secretory pathway of a host cell, a secretory signal sequence (also known as a leader sequence, prepro sequence or pre sequence) is provided in the expression vector. The secretory signal sequence may be that of the receptor, or may be derived from another secreted protein (e.g., t-PA) or synthesized de novo. The secretory signal sequence is joined to the soluble FcγRIA DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are preferred hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981; Graham and Van der Eb, Virology 52:456, 1973), electroporation (Neumann et al., EMBO J. 1:841-845, 1982), DEAE-dextran mediated transfection (Current Protocols in Molecular Biology, (Ausubel et al. eds., John Wiley and Sons, Inc., NY, 1987)), and liposome-mediated transfection (Hawley-Nelson et al., Focus 15:73, 1993; Ciccarone et al., Focus 15:80, 1993), which are incorporated herein by reference. The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. Transformation of insect cells and production of foreign polypeptides therein is disclosed by Guarino et al., U.S. Pat. No. 5,162,222; Bang et al., U.S. Pat. No. 4,775,624; and WIPO publication WO 94/06463, which are incorporated herein by reference. The use of Agrobacterium rhizogenes as a vector for expressing genes in plant cells has been reviewed by Sinkar et al., J. Biosci. (Bangalore) 11:47-58, 1987.

Fungal cells, including yeast cells, and particularly cells of the genus Saccharomyces, can also be used within the present invention, such as for producing receptor fragments or polypeptide fusions. Methods for transforming yeast cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. (See also U.S. Pat. Nos. 4,990,446; 5,063,154; 5,139,936 and 4,661,454.) Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. (See, e.g., Gleeson et al., J. Gen. Microbiol. 132:3459-3465, 1986; Cregg, U.S. Pat. No. 4,882,279.) Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Within one aspect of the present invention, a FcγRIA polypeptide of the present invention is produced by a cultured cell, and the cell is used to screen for antagonists of IgG. To summarize this approach, a cDNA or gene encoding the FcγRIA receptor is combined with other genetic elements required for its expression (e.g., a transcription promoter), and the resulting expression vector is inserted into a host cell. Cells that express the DNA and produce functional receptor are selected and used within a variety of screening systems.

Mammalian cells suitable for use in expressing FcγRIA receptors, including the soluble FcγRIA polypeptides of the invention include the preferred cell lines of this type are the human TF-1 cell line (ATCC number CRL-2003) and the AML-193 cell line (ATCC number CRL-9589), which are GM-CSF-dependent human leukemic cell lines and BaF3 (Palacios and Steinmetz, Cell 41: 727-734, (1985)) which is an IL-3 dependent murine pre-B cell line. Other cell lines include BHK, COS-1 and CHO cells.

Suitable host cells can be engineered to produce the necessary receptor subunits or other cellular component needed for the desired cellular response. This approach is advantageous because cell lines can be engineered to express receptor subunits from any species, thereby overcoming potential limitations arising from species specificity. Species orthologs of the human receptor cDNA can be cloned and used within cell lines from the same species, such as a mouse cDNA in the BaF3 cell line. Cell lines that are dependent upon one hematopoietic growth factor, such as GM-CSF or IL-3.

Cells expressing functional receptor are used within screening assays. A variety of suitable assays are known in the art. These assays are based on the detection of a biological response in a target cell. One such assay is a cell proliferation assay. Cells are cultured in the presence or absence of a test compound, and cell proliferation is detected by, for example, measuring incorporation of tritiated thymidine or by calorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. A preferred promoter element in this regard is a serum response element, or SRE. (See, e.g., Shaw et al., Cell 56:563-572, 1989.) A preferred such reporter gene is a luciferase gene. (See de Wet et al., Mol. Cell. Biol. 7:725, 1987.) Expression of the luciferase gene is detected by luminescence using methods known in the art. (See, e.g., Baumgartner et al., J. Biol. Chem. 269:29094-29101, 1994; Schenborn and Goiffin, Promega Notes 41:11, 1993.) Luciferase activity assay kits are commercially available from, for example, Promega Corp., Madison, Wis. Target cell lines of this type can be used to screen libraries of chemicals, cell-conditioned culture media, fungal broths, soil samples, water samples, and the like. For example, a bank of cell-conditioned media samples can be assayed on a target cell to identify cells that produce ligand. Positive cells are then used to produce a cDNA library in a mammalian expression vector, which is divided into pools, transfected into host cells, and expressed. Media samples from the transfected cells are then assayed, with subsequent division of pools, re-transfection, subculturing, and re-assay of positive cells to isolate a cloned cDNA encoding the ligand.

The soluble FcγRIA polypeptides of the invention can be prepared by expressing a truncated DNA encoding the extracellular domain, for example, a polypeptide which contains residues 16-282 or 16-292 of SEQ ID NO:2 or the corresponding region of a non-human receptor. It is preferred that the extracellular domain polypeptides be prepared in a form substantially free of transmembrane and intracellular polypeptide segments. To direct the export of the receptor domain from the host cell, the receptor DNA is linked to a second DNA segment encoding a secretory peptide, such as FcγRIA's native signal sequence (described in the Examples below). Other signal sequences that could be used include otPA pre-pro secretion, CD33 signal sequence or human growth hormone signal sequence. To facilitate purification of the secreted receptor domain, a C-terminal extension, such as a poly-histidine tag, substance P, Flag™ peptide (Hopp et al., Biotechnology 6:1204-1210, 1988; available from Eastman Kodak Co., New Haven, Conn.) or another polypeptide or protein for which an antibody or other specific binding agent is available, can be fused to the receptor polypeptide.

### VI. Production of FcγRIA Fusion Proteins and Conjugates

In an alternative approach, a soluble FcγRIA polypeptide (i.e. the extracellular domain of a FcγRIA receptor) can be expressed as a fusion with immunoglobulin heavy chain constant regions, typically an F_{C} fragment, which contains two constant region domains and a hinge region but lacks the variable region (See, Sledziewski, A Z et al., U.S. Pat. Nos. 6,018,026 and 5,750,375). The soluble FcγRIA polypeptides of the present invention include such fusions. Such fusions are typically secreted as multimeric molecules wherein the Fc portions are disulfide bonded to each other and two polypeptides are arrayed in closed proximity to each other. Fusions of this type can be used to affinity purify the cognate ligand from solution, as an in vitro assay tool, to block signals in vitro by specifically titrating out ligand, and as antagonists in vivo by administering them parenterally to bind circulating ligand and clear it from the circulation. Circulating molecules bind ligand and are cleared from circulation by normal physiological processes. For use in assays, the chimeras are bound to a support via the Fc region and used in an ELISA format.

The present invention further provides a variety of other polypeptide fusions and related proteins comprising one or more polypeptide fusions. For example, a soluble FcγRIA polypeptide can be prepared as a fusion to a dimerizing protein as disclosed in U.S. Pat. Nos. 5,155,027 and 5,567,584. Preferred dimerizing proteins in this regard include immunoglobulin constant region domains, e.g., IgGγ1, and the human κ light chain. Immunoglobulin-soluble FcγRIA polypeptide fusions can be expressed in genetically engineered cells to produce a variety of such receptor analogs. Auxiliary domains can be fused to soluble FcγRIA receptor to target them to specific cells, tissues, or macromolecules (e.g., collagen, or cells expressing other Fc receptors). Thus, the soluble FcγRIA receptor polypeptides of the invention can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The present invention also contemplates chemically modified FcγRIA compositions, in which a FcγRIA polypeptide is linked with a polymer. Illustrative FcγRIA polypeptides are soluble polypeptides that lack a functional transmembrane domain, such as a polypeptide consisting of amino acid residues 16-282 or 16-292 of SEQ ID NO:2. Typically, the polymer is water soluble so that the FcγRIA conjugate does not precipitate in an aqueous environment, such as a physiological environment. An example of a suitable polymer is one that has been modified to have a single reactive group, such as an active ester for acylation, or an aldehyde for alkylation. In this way, the degree of polymerization can be controlled. An example of a reactive aldehyde is polyethylene glycol propionaldehyde, or mono-(C1-C10) alkoxy, or aryloxy derivatives thereof (see, for example, Harris, et al., U.S. Pat. No. 5,252,714). The polymer may be branched or unbranched. Moreover, a mixture of polymers can be used to produce FcγRIA conjugates.

FcγRIA conjugates used for therapy can comprise pharmaceutically acceptable water-soluble polymer moieties. Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, PEG propionaldehyde, bis-succinimidyl carbonate PEG, propylene glycol homopolymers, a polypropylene oxide/ethylene oxide co-polymer, polyoxyethylated polyols (e.g., glycerol), polyvinyl alcohol, dextran, cellulose, or other carbohydrate-based polymers. Suitable PEG may have a molecular weight from about 600 to about 60,000, including, for example, 5,000, 12,000, 20,000 and 25,000. A FcγRIA conjugate can also comprise a mixture of such water-soluble polymers.

One example of a FcγRIA conjugate comprises a FcγRIA moiety and a polyalkyl oxide moiety attached to the N-terminus of the FcγRIA moiety. PEG is one suitable polyalkyl oxide. As an illustration, FcγRIA can be modified with PEG, a process known as “PEGylation.” PEGylation of FcγRIA can be carried out by any of the PEGylation reactions known in the art (see, for example, EP 0 154 316, Delgado et al., Critical Reviews in Therapeutic Drug Carrier Systems 9:249 (1992), Duncan and Spreafico, Clin. Pharmacokinet. 27:290 (1994), and Francis et al., Int J Hematol 68:1 (1998)). For example, PEGylation can be performed by an acylation reaction or by an alkylation reaction with a reactive polyethylene glycol molecule. In an alternative approach, FcγRIA conjugates are formed by condensing activated PEG, in which a terminal hydroxy or amino group of PEG has been replaced by an activated linker (see, for example, Karasiewicz et al., U.S. Pat. No. 5,382,657).

PEGylation by acylation typically requires reacting an active ester derivative of PEG with a FcγRIA polypeptide. An example of an activated PEG ester is PEG esterified to N-hydroxysuccinimide. As used herein, the term “acylation” includes the following types of linkages between FcγRIA and a water soluble polymer: amide, carbamate, urethane, and the like. Methods for preparing PEGylated FcγRIA by acylation will typically comprise the steps of (a) reacting a FcγRIA polypeptide with PEG (such as a reactive ester of an aldehyde derivative of PEG) under conditions whereby one or more PEG groups attach to FcγRIA, and (b) obtaining the reaction product(s). Generally, the optimal reaction conditions for acylation reactions will be determined based upon known parameters and desired results. For example, the larger the ratio of PEG:FcγRIA, the greater the percentage of polyPEGylated FcγRIA product.

The product of PEGylation by acylation is typically a polyPEGylated FcγRIA product, wherein the lysine ε-amino groups are PEGylated via an acyl linking group. An example of a connecting linkage is an amide. Typically, the resulting FcγRIA will be at least 95% mono-, di-, or tri-pegylated, although some species with higher degrees of PEGylation may be formed depending upon the reaction conditions. PEGylated species can be separated from unconjugated FcγRIA polypeptides using standard purification methods, such as dialysis, ultrafiltration, ion exchange chromatography, affinity chromatography, and the like.

PEGylation by alkylation generally involves reacting a terminal aldehyde derivative of PEG with FcγRIA in the presence of a reducing agent. PEG groups can be attached to the polypeptide via a —CH₂—NH group.

Derivatization via reductive alkylation to produce a monoPEGylated product takes advantage of the differential reactivity of different types of primary amino groups available for derivatization. Typically, the reaction is performed at a pH that allows one to take advantage of the pKa differences between the ε-amino groups of the lysine residues and the α-amino group of the N-terminal residue of the protein. By such selective derivatization, attachment of a water-soluble polymer that contains a reactive group such as an aldehyde, to a protein is controlled. The conjugation with the polymer occurs predominantly at the N-terminus of the protein without significant modification of other reactive groups such as the lysine side chain amino groups. The present invention provides a substantially homogenous preparation of FcγRIA monopolymer conjugates.

Reductive alkylation to produce a substantially homogenous population of monopolymer FcγRIA conjugate molecule can comprise the steps of: (a) reacting a FcγRIA polypeptide with a reactive PEG under reductive alkylation conditions at a pH suitable to permit selective modification of the α-amino group at the amino terminus of the FcγRIA, and (b) obtaining the reaction product(s). The reducing agent used for reductive alkylation should be stable in aqueous solution and able to reduce only the Schiff base formed in the initial process of reductive alkylation. Illustrative reducing agents include sodium borohydride, sodium cyanoborohydride, dimethylamine borane, trimethylamine borane, and pyridine borane.

For a substantially homogenous population of monopolymer FcγRIA conjugates, the reductive alkylation reaction conditions are those that permit the selective attachment of the water-soluble polymer moiety to the N-terminus of FcγRIA. Such reaction conditions generally provide for pKa differences between the lysine amino groups and the α-amino group at the N-terminus. The pH also affects the ratio of polymer to protein to be used. In general, if the pH is lower, a larger excess of polymer to protein will be desired because the less reactive the N-terminal α-group, the more polymer is needed to achieve optimal conditions. If the pH is higher, the polymer: FcγRIA need not be as large because more reactive groups are available. Typically, the pH will fall within the range of 3 to 9, or 3 to 6. This method can be employed for making FcγRIA-comprising homodimeric, heterodimeric or multimeric soluble receptor conjugates.

Another factor to consider is the molecular weight of the water-soluble polymer. Generally, the higher the molecular weight of the polymer, the fewer number of polymer molecules which may be attached to the protein. For PEGylation reactions, the typical molecular weight is about 2 kDa to about 100 kDa, about 5 kDa to about 50 kDa, or about 12 kDa to about 25 kDa. The molar ratio of water-soluble polymer to FcγRIA will generally be in the range of 1:1 to 100:1. Typically, the molar ratio of water-soluble polymer to FcγRIA will be 1:1 to 20:1 for polyPEGylation, and 1:1 to 5:1 for monoPEGylation.

General methods for producing conjugates comprising a polypeptide and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997)). This method can be employed for making FcγRIA-comprising homodimeric, heterodimeric or multimeric soluble receptor conjugates.

The present invention contemplates compositions comprising a peptide or polypeptide, such as a soluble receptor or antibody described herein. Such compositions can further comprise a carrier. The carrier can be a conventional organic or inorganic carrier. Examples of carriers include water, buffer solution, alcohol, propylene glycol, macrogol, sesame oil, corn oil, and the like.

### VII. Isolation of FcγRIA Polypeptides

The polypeptides of the present invention can be purified to at least about 80% purity, to at least about 90% purity, to at least about 95% purity, or greater than 95%, such as 96%, 97%, 98%, or greater than 99% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. In certain preparations, purified polypeptide is substantially free of other polypeptides, particularly other polypeptides of animal origin.

Fractionation and/or conventional purification methods can be used to obtain preparations of FcγRIA purified from natural sources (e.g., human tissue sources), synthetic FcγRIA polypeptides, and recombinant FcγRIA polypeptides and fusion FcγRIA polypeptides purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method for polypeptide isolation and purification is a matter of routine design and is determined in part by the properties of the chosen support. See, for example, Affinity Chromatography: Principles & Methods (Pharmacia LKB Biotechnology 1988), and Doonan, Protein Purification Protocols (The Humana Press 1996).

Additional variations in FcγRIA isolation and purification can be devised by those of skill in the art. For example, anti-FcγRIA antibodies can be used to isolate large quantities of protein by immunoaffinity purification.

The polypeptides of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1 (1985)). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (M. Deutscher (ed.), Meth. Enzymol. 182:529, 1990). Within additional embodiments of the invention, a fusion of the polypeptide of interest and an affinity tag (e.g., maltose-binding protein, an immunoglobulin domain) may be constructed to facilitate purification.

FcγRIA polypeptides or fragments thereof may also be prepared through chemical synthesis, as described above. FcγRIA polypeptides may be monomers or multimers; glycosylated or non-glycosylated; PEGylated or non-PEGylated; and may or may not include an initial methionine amino acid residue.

A preferred assay system employing a ligand-binding receptor fragment, such as the soluble FcγRIA polypeptide of the present invention, uses a commercially available biosensor instrument (BIAcore™, Pharmacia Biosensor, Piscataway, N.J.), wherein the receptor fragment is immobilized onto the surface of a receptor chip. Use of this instrument is disclosed by Karlsson (J. Immunol. Methods 145:229-240, 1991) and Cunningham and Wells (J. Mol. Biol. 234:554-563, 1993). A receptor fragment is covalently attached, using amine or sulfhydryl chemistry, to dextran fibers that are attached to gold film within the flow cell. A test sample is passed through the cell. If ligand (i.e. IgG) is present in the sample, it will bind to the immobilized receptor polypeptide, causing a change in the refractive index of the medium, which is detected as a change in surface plasmon resonance of the gold film. This system allows the determination of on- and off-rates, from which binding affinity can be calculated, and assessment of stoichiometry of binding.

The soluble FcγRIA polypeptides can also be used within other assay systems known in the art. Such systems include Scatchard analysis for determination of binding affinity (see Scatchard, Ann. NY Acad. Sci. 51: 660-672, 1949) and calorimetric assays (see Cunningham et al., Science 253:545-548, 1991; Cunningham et al., Science 254:821-825, 1991). The soluble FcγRIA polypeptides can also be used to block the precipitation of antigen-antibody immune complexes and can also be used to block cytokine secretion from mast cells in cell culture.

Moreover, soluble FcγRIA polypeptides can be used as a “ligand sink,” i.e., antagonist, to bind ligand (i.e., IgG or immune complexes) in vivo or in vitro in therapeutic or other applications where the presence of the ligand, or ligand signaling is not desired. For example, in autoimmune diseases, a soluble FcγRIA polypeptide can be used as a direct antagonist of IgG in vivo, and may aid in reducing progression and symptoms associated with the disease (and inflammation), and can be used in conjunction with other therapies (e.g., other anti-inflammatories) to enhance the effect of the therapy in reducing progression and symptoms, and preventing relapse.

Antibodies to FcγRIA polypeptides (and in particular to the soluble FcγRIA polypeptides of the invention) may be used for tagging cells that express FcγRIA receptors; for isolating soluble FcγRIA polypeptides by affinity purification; for diagnostic assays for determining circulating levels of soluble FcγRIA polypeptides; for detecting or quantitating FcγRIA receptor as marker of underlying pathology or disease; in analytical methods employing FACS; for screening expression libraries; and for generating anti-idiotypic antibodies; and as neutralizing antibodies or as antagonists to block IgG binding to Fc receptors in vitro and in vivo.

Soluble FcγRIA polypeptides can also be used to prepare antibodies that bind to epitopes, peptides, or polypeptides contained within the antigen. The FcγRIA polypeptide or a fragment thereof serves as an antigen (immunogen) to inoculate an animal and elicit an immune response. One of skill in the art would recognize that antigens or immunogenic epitopes can consist of stretches of amino acids within a longer polypeptide, from about 10 amino acids and up to about the entire length of the polypeptide or longer depending on the polypeptide. Suitable antigens include a soluble FcγRIA polypeptide comprising amino acid residues 16-282 or 16-292 of SEQ ID NO:2 or a fragment thereof. Preferred peptides to use as antigens are hydrophilic peptides such as those predicted by one of skill in the art from a hydrophobicity plot, determined for example, from a Hopp/Woods hydrophilicity profile based on a sliding six-residue window, with buried G, S, and T residues and exposed H, Y, and W residues ignored, or from a Jameson-Wolf plot of amino acid residues 16-282 or 16-292 of SEQ ID NO:2 using a DNA*STAR program. In addition, conserved motifs, and variable regions between conserved motifs of zcytor11 soluble receptor are suitable antigens. Moreover, corresponding regions of the mouse FcγRIA receptor can be used to generate antibodies against the mouse receptor. In addition, antibodies generated from this immune response can be isolated and purified as described herein. Methods for preparing and isolating polyclonal and monoclonal antibodies are well known in the art. See, e.g., Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a soluble FcγRIA polypeptide or a fragment thereof. The immunogenicity of a FcγRIA polypeptide may be increased through the use of an adjuvant, such as alum (aluminum hydroxide) or Freund's complete or incomplete adjuvant. Polypeptides useful for immunization also include fusion polypeptides, such as fusions of FcγRIA polypeptide or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Antibodies are considered to be specifically binding if: 1) they exhibit a threshold level of binding activity, and 2) they do not significantly cross-react with related polypeptide molecules. A threshold level of binding is determined if anti-FcγRIA antibodies described herein bind to a FcγRIA polypeptide with an affinity at least 10-fold greater than the binding affinity to a control polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, Ann. NY Acad. Sci. 51:660-672, 1949).

A variety of assays known to those skilled in the art can be utilized to detect antibodies that bind to FcγRIA polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus variant FcγRIA polypeptides (such as those described herein).

### VIII. Uses of Soluble-FcγRIA Antibodies to Antagonize IgG Binding to Fc Receptors

The FcγRIA polypeptides of the invention have a large number of uses. These FcγRIA polypeptides can be used for tagging cells; for isolating homolog polypeptides by affinity purification; they can be directly or indirectly conjugated to drugs, toxins, radionuclides and the like. The FcγRIA polypeptides can also be used in analytical methods such as for screening expression libraries and neutralizing activity, e.g., for binding, blocking, inhibiting, reducing, antagonizing or neutralizing interaction between IgG and Fc receptors. The FcγRIA polypeptides can also be used for diagnostic assays. The FcγRIA polypeptides of the invention, such as soluble FcγRIA, can also act as “antagonists” to block or inhibit binding of IgG (e.g. to ligand) and signal transduction in vitro and in vivo. The FcγRIA polypeptides (e.g., soluble polypeptides comprising amino acid residues 16-282 or 16-292 of SEQ ID NO:2) act specifically against IgG and can inhibit IgG binding to an Fcγ receptor, and are thus useful for inhibiting IgG and Fcγ receptor activity. Moreover, the antagonistic and binding activity of the soluble FcγRIA polypeptides of the present invention can be assayed in proliferation, luciferase, or binding assays in the presence of IgG respectively, and other biological or biochemical assays described herein.

The soluble FcγRIA polypeptides of the invention are useful for modulating an immune response by binding IgG and, thus, inhibiting the binding of IgG with endogenous receptor (i.e., Fcγ receptors). Accordingly, the present invention includes the use of FcγRIA polypeptides, including soluble FcγRIA polypeptides, to treat a subject with inflammation or having an immune disease or disorder. Suitable subjects include mammals, such as humans. The soluble FcγRIA polypeptides of the invention may be used, therefore, for inhibiting the inflammatory effects of IgG and/or immune complexes in vivo, for therapeutic use against SLE, cryoglobulinemia, autoimmune thrombocytopenias (ITP and TTP), adult dermatomyositis, hepatitis-C-associated cryoglobulinemia, hepatitis-B-associated polyarteritis nodosa, Guillian-Barre syndrome, Goodpasture's syndrome, chronic inflammatory demyelinating polyneuropathies, anti-phospholipid antibody syndrome, vasculitis, uveitis, serum sickness, pemphigus (e.g., pemphigus vulgaris), diseases associated with exogenous antigens, psoriasis, atopic dermatitis, inflammatory skin conditions, endotoxemia, arthritis, asthma, IBD, colitis, psoriatic arthritis, rheumatoid arthritis, or other IgG- or immune complex-mediated inflammatory conditions; and as antagonists to bind, block, inhibit, reduce, or antagonize IgG or Fc receptor function, or to bind, block, inhibit, reduce, antagonize, or neutralize IgG and/or immune complex activity in vitro and in vivo.

The FcγRIA polypeptides herein may also be directly or indirectly conjugated to drugs, toxins, radionuclides and the like, and these conjugates used for in vivo diagnostic or therapeutic applications. For instance, the FcγRIA polypeptides of the invention can be used to identify or treat tissues or organs in need thereof. More specifically, the soluble FcγRIA polypeptides can be coupled to detectable or cytotoxic molecules and delivered to a mammal experiencing inflammation or immune disease.

Suitable detectable molecules may be directly or indirectly attached to the FcγRIA polypeptides herein. Suitable detectable molecules include radionuclides, enzymes, substrates, cofactors, inhibitors, fluorescent markers, chemiluminescent markers, magnetic particles and the like. Suitable cytotoxic molecules may be directly or indirectly attached to the polypeptide, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or indirectly attached through means of a chelating moiety, for instance). The FcγRIA polypeptides may also be conjugated to cytotoxic drugs, such as adriamycin. For indirect attachment of a detectable or cytotoxic molecule, the detectable or cytotoxic molecule can be conjugated with a member of a complementary/anticomplementary pair, where the other member is bound to the FcγRIA polypeptide. For these purposes, biotin/streptavidin is an exemplary complementary/anticomplementary pair.

Moreover, as shown herein, a soluble FcγRIA polypeptide completely blocked immune complex precipitation (described in detail in the Examples below). As also shown herein, a soluble FcγRIA polypeptide blocked the binding and signaling of immune complexes (described in detail in the Examples below). These findings suggest that soluble FcγRIA is a potent therapeutic that can be used to treat autoimmune disease and inflammation. Thus, within preferred embodiments, the soluble FcγRIA polypeptide is a monomer or homodimer that binds to, blocks, inhibits, reduces, antagonizes, or neutralizes IgG in vivo. Within another preferred embodiment, the soluble FcγRIA polypeptide is a monomer or homodimer that blocks the binding and signaling of immune complexes. In particular variations, the FcγRIA polypeptide comprises amino acid residues 16-282 or 16-292 of SEQ ID NO:2.

As stated above, the FcγRIA polypeptides described herein can have beneficial use as antagonists of IgG or immune complexes and, thus, as therapeutics against IgG- or immune complex-mediated human diseases. Accordingly, the present invention provides such novel antagonists and uses thereof, including soluble FcγRIA polypeptides such as soluble polypeptides comprising amino acid residues 16-282 or 16-292 of SEQ ID NO:2.

As described above, Fc receptors such as FcγRIA, have a clear functional role in the capture and clearance of immune complexes, as well as antibody-dependent cell cytotoxicity (ADCC), and cytokine/inflammatory mediator release. Thus, the soluble FcγRIA polypeptides of the invention have therapeutic uses in, e.g., cancer, infectious disease, and autoimmune disease. For example, methods of treating, modulating, reducing, or suppressing IgG-induced or immune complex-induced inflammation comprises administering to a mammal with inflammation an amount of a composition of soluble FcγRIA sufficient to reduce IgG-mediated or immune complex-mediated inflammation. Experimental evidence described herein shows that the soluble FcγRIA polypeptides of the invention have anti-inflammatory effects.

As indicated in the discussion above and the examples below, IgG and Fc receptors (e.g., FcγRIA) are involved in the pathology of inflammation. In certain aspects, the present invention is a method for treating inflammation by administering agents that bind, block, inhibit, reduce, antagonize, or neutralize IgG or immune complexes. Thus, particular embodiments of the present invention are directed toward the use of soluble FcγRIA polypeptides as antagonists in IgG-mediated inflammatory and immune diseases or conditions such as, for example, systemic lupus erythematosus (SLE); lupus (including nephritis, non-renal, discoid, alopecia); cryoglobulinemia; mixed connective tissue disease; autoimmune thrombocytopenias (idiopathic thrombocytopenic purpura (ITP); thrombotic throbocytopenic purpura (TTP)); Sjogren's syndrome; adult dermatomyositis; hepatitis-C-associated cryoglobulinemia; hepatitis-B-associated polyarteritis nodosa; Guillian-Barre syndrome; Goodpasture's syndrome; chronic inflammatory demyelinating polyneuropathies; anti-phospholipid antibody syndrome; vasculitis; uveitis; serum sickness; diseases associated with exogenous antigens; arthritis (rheumatoid arthritis, juvenile rheumatoid arthritis, psoriatic arthritis); psoriasis; atopic dermatitis; inflammatory skin conditions; responses associated with inflammatory bowel disease (IBD) (Crohn's disease, ulcerative colitis); diverticulosis; asthma; pancreatitis; type I juvenile onset) diabetes (IDDM); pancreatic cancer; pancreatitis; Grave's Disease; chronic autoimmune urticaria; polymyositis/dermatomyositis; toxic epidermal necrolysis; systemic scleroderma and sclerosis; respiratory distress syndrome; adult respiratory distress syndrome (ARDS); meningitis; allergic rhinitis; encephalitis; colitis; glomerulonephritis; IgG-mediated allergic conditions; atherosclerosis, autoimmune myocarditis; multiple sclerosis; allergic encephalomyelitis; sarcoidosis, granulomatosis including Wegener's granulomatosis; agranulocytosis; aplastic anemia; Coombs positive anemia; Diamond Blackfan anemia; immune hemolytic anemia including autoimmune hemolytic anemia (AIHA); pernicious anemia; pure red cell aplasia (PRCA); Factor VIII deficiency; hemophilia A; autoimmune neutropenia; pancytopenia; leucopenia; diseases involving leukocyte diapedesis; CNS inflammatory disorders; multiple organ injury syndrome; myasthenia gravis; anti-glomerular basement membrane disease; Bechet disease; Castleman's syndrome; Lambert-Eaton Myasthenic Syndrome; Reynaud's syndrome; Stevens-Johnson syndrome; bone marrow transplant rejection; solid organ transplant rejection (including pretreatment for high panel reactive antibody titers); graft-versus-host disease (GVHD); pemphigoid bullous; pemphigus (all including vulgaris, foliaceus); autoimmune polyendocrinopathies; Reiter's disease; stiff-man syndrome; immune complex nephritis; autoimmune disease of the testis and ovary including autoimmune orchitis and oophoritis; primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, autoimmune polyglandular syndromes (or polyglandular endocrinopathy syndromes); autoimmune hepatitis; Lymphoid interstitial pneumonitis (HIV); bronchiolitis obliterans (non-transplant) vs NSIP, large vessel vasculitis (including polymyalgia rheumatica and giant cell (Takayasu's) arteritis); medium vessel vasculitis (including Kawasaki's Disease and polyarteritis nodosa); ankylosing spondylitis; rapidly progressive glomerulonephritis; primary biliary cirrhosis; celiac sprue (gluten enteropathy); ALS; coronary artery disease; or other instances where inhibition of IgG or immune complexes is desired.

Asthma, allergy, and other atopic disease may be treated with a soluble FcγRIA polypeptide of the invention (e.g., a soluble polypeptide comprising amino acid residues 16-282 or 16-292 of SEQ ID NO:2) to inhibit the immune response. The polypeptides of the present invention may also be used to treat diseases of the pancreas, kidney, pituitary, and neuronal cells. IDDM, NIDDM, pancreatitis, and pancreatic carcinoma may benefit. The FcγRIA polypeptides of the invention may also be used for treatment of cancer where a soluble FcγRIA polypeptide inhibits cancer growth and targets immune-mediated killing. The FcγRIA polypeptides of the invention may also be used to treat nephropathies such as glomerulosclerosis, membranous neuropathy, amyloidosis (which also affects the kidney among other tissues), renal arteriosclerosis, glomerulonephritis of various origins, fibroproliferative diseases of the kidney, as well as kidney dysfunction associated with SLE, IDDM, type II diabetes (NIDDM), renal tumors, and other diseases.

The FcγRIA polypeptides of the invention may also be used to treat psychological disorders associated with deposition of immune complexes with the choroids plexus of the brain. Such deposition, for example, may underlie the central and peripheral nervous system manifestations of diseases such as Systemic Lupus Erythematosus. In some patients, these manifestations are a major cause of morbidity and mortality and include cognitive dysfunction, particularly difficulties with memory and reasoning, psychosis, headaches, and seizures. As another example, deposition of immune complexes within the choroid plexus may be responsible for the peripheral neuropathy seen in essential mixed cryoglobulinemia. (See Harrison's Principles of Internal Medicine (Kasper et al. eds., McGraw-Hill, New York 2005).)

As described herein, soluble FcγRIA polypeptides of the present invention are useful as antagonists of IgG or IgG-containing immune complex binding to Fc receptors. Such antagonistic effects can be achieved by direct neutralization or binding of IgG. In addition to antagonistic uses, the soluble receptors of the present invention can bind IgG or IgG-containing immune complexes and act as carrier proteins, in order to transport the ligand to different tissues, organs, and cells within the body. As such, the soluble FcγRIA polypeptides of the present invention can be fused or coupled to molecules, polypeptides or chemical moieties that direct the soluble-receptor-ligand complex to a specific site, such as a tissue, specific immune cell, or tumor. For example, in acute infection or some cancers, benefit may result from induction of inflammation and local acute phase response proteins.

Accordingly, the FcγRIA polypeptides of the invention have therapeutic potential for a wide variety of IgG-mediated inflammatory diseases. Inflammation—a protective response by an organism to fend off an invading agent—is a cascading event that involves many cellular and humoral mediators. On one hand, suppression of inflammatory responses can leave a host immunocompromised; if left unchecked, however, inflammation can lead to serious complications including, for example, chronic inflammatory diseases (e.g., psoriasis, arthritis, rheumatoid arthritis, multiple sclerosis, inflammatory bowel disease, and the like), septic shock, and multiple organ failure. Importantly, these diverse disease states share common inflammatory mediators. The collective diseases that are characterized by inflammation have a large impact on human morbidity and mortality. The studies described herein show, inter alia, the ability of soluble FcγRIA to block the binding and signaling of immune complexes, as well as the ability of soluble FcγRIA to treat immune complex-mediated disease. Thus, the FcγRIA polypeptides of the invention have therapeutic potential for a vast number of human and animal diseases such as, for example, the IgG- and immune complex-mediated diseases discussed herein. Exemplary diseases amenable to treatment using soluble FcγRIA are further described in Sections VIII(A) and VIII(B), infra.

A. Immune-Complex-Mediated Diseases

The binding of an antigen with its cognate antibody generates immune complexes, and deposition of these immune complexes within tissues is the pathogenic mechanism underlying a variety of autoimmune diseases (see Jancar and Crespo, Trends Immunol. 26:48-55, 2005). These diseases include the connective tissue autoimmune diseases such as systemic lupus erythematosus (SLE), dermatomyositis, rheumatoid arthritis, Sjogren's syndrome, and mixed connective tissue disease; diseases of diverse etiology such as cryoglobulinemia, polyarteritis nodosa, and the anti-phospholipid syndrome; as well as diseases associated with exogenous antigens including bacterial, viral, and parasitic infections, diseases associated with organic dusts, and serum sickness type of diseases including passive immunotherapy for infection, venomous snake bites, and drug hypersensitivity. While each of these conditions is caused by and exhibits specific antigen-antibody pairs, the mechanism for tissue damage is similar: the formation of circulating immune complexes, followed by their deposition within tissues (see Jancar and Crespo, supra). Antigen-antibody complexes can damage tissues by triggering inflammation, a process mediated in part through the binding of immune complexes to cell surface Fc gamma receptors and by their ability to fix complement.

In the normal situation, immune complexes are cleared by phagocytic cells of the reticuloendothelial system. In some instances, however, immune complexes accumulate and deposit in tissues, causing type III hypersensitivity reactions. (See Jancar and Crespo, supra.) When immune complexes form in the blood, deposition can occur at sites removed from the site of antigen entry. Complex deposition is routinely observed, e.g., on blood vessel walls, in the synovial membranes of joints, on the glomerular basement membrane of the kidney, and on the choroid plexus of the brain, sites where filtration of plasma occurs. (See Jancar and Crespo, supra.) This is the reason for the high incidence of arthritis, vasculitis, and glomerulonephritis observed in immune complex-mediated diseases, such as cryoglobulinemia.

Following their deposition within tissues, immune complexes bind to cell surface FcγR via the Fc domain of IgG. As previously noted, FcγR play a crucial role as a link between the humoral and cellular arms of the immune system (see Cohen-Solal et al., Immunol. Lett. 92:199-205, 2004; Hogarth et al., Curr. Opin. Immunol. 14:798-802, 2002; Nakamura et al., Expert Opin. Ther. Targets 9:169-190, 2005; Nimmerjahn, Springer Semin. Immunopathol. 28:305-319, 2006). Ligation of these cell surface receptors by the Fc portion of IgG can trigger a variety of immune effector functions such as antigen presentation, antibody dependent cellular cytotoxicity (ADCC), phagocytosis, and the release of inflammatory mediators. The three main classes of Fcγ receptors-FcγRI, FcγRII, and FcγRIII—are expressed within specific and overlapping subsets of cells of the human immune system, expression patterns that account for their diverse roles in immune homeostasis (see Nakamura et al., supra). With the exception of FcγRI, which exhibits a high affinity for monomeric IgG, the other subclasses of FcγRs are low affinity IgG receptors. (See Cohen-Solal et al., supra; Hogarth et al., supra.) However, these cellular receptors bind antigen-antibody immune complexes (IC) with high avidity, through multiple Fc:FcγR interactions. This property is thought to allow cells expressing FcγRII and/or FcγRIII to sample their extracellular environment and respond appropriately to IC in the face of saturating amounts of monomeric IgG. (See Hogarth et al., supra.)

As part of a screening effort to identify soluble receptors demonstrating this ability, the soluble extracellular domains of each of the human FcγR were expressed in CHO cells and purified to homogeneity from their conditioned media. While each of the rh-FcγR reduced immune complex-mediated inflammatory events in several in vitro systems, only the high affinity receptor, FcγRIA, produced consistent reductions in inflammation in the cutaneous reverse passive Arthus reaction in mice. This result was unexpected in that FcγRIA, as a high affinity receptor for monomeric IgG, was generally expected to be saturated with circulating monomeric IgG in vivo and thus unavailable for binding to IC. The observation that systemic delivery of FcγRIA also abolished inflammation in the murine collagen antibody-induced model of arthritis suggests that FcγRIA may be a novel therapy for treating immune complex-mediated diseases.

Accordingly, by blocking the binding of immune complexes to cell surface Fc gamma receptors, the FcγRIA polypeptides of the invention can reduce inflammatory cytokine secretion and reduce infiltration of inflammatory cell types such as neutrophils. As demonstrated by studies described herein, FcγRIA polypeptides blocked the precipitation of antigen antibody immune complexes and inhibited immune complex-mediated cytokine secretion by mast cells (see Examples 15 and 16, infra). In studies in mice, moreover, FcγRIA polypeptides reduced edema and neutrophil infiltration in the cutaneous reverse passive Arthus reaction and reduced paw inflammation in the collagen antibody-induced arthritis model and, moreover, in collagen-induced arthritis in mice. (See Examples 15-17 and 19, infra.) Thus, the FcγRIA polypeptides can be used in the treatment of various immune complex-mediated diseases in humans or other non-human species.

1. Cryoblobulinemia

The term cryoglobulinemia refers to the presence in serum of one (monoclonal cryoglobulinemia) or more (mixed cryoglobulinemia) immunoglobulins that reversibly precipitate at temperatures below 37° C. (See Meltzer and Franklin, Am. J. Med. 40:828-836, 1996; Dammacco et al., Eur. J. Clin. Invest. 31:628-638, 2001; Sansonno et al., Rheumatology (Oxford) 46:572-578, 2007). The mechanism of cryoprecipitation is obscure but may involve alterations in Ig structure, self-association of Ig Fc domains, and/or IgM rheumatoid factor activity. (See Sansonno and Dammacco, Lancet Infect. Dis. 5:227-236, 2005.) Cryoglobulinemia is classified into three subgroups (see Dammacco et al., supra): Type I is composed of a single monoclonal Ig; Type II is composed of a mixture of monoclonal IgM and polyclonal IgG; and Type III is a mixture of polyclonal IgM/IgG. Cryoglobulinemia types I, II, and III account for approximately 10-15%, 50-60%, and 30-40%, of all people with serum cryoprecipitates, respectively. (See Dammacco et al., supra; Sansonno et al., supra.)

Patients with cryogobulinemia present most often with a clinical triad of purpura, weakness, and arthralgias, as well as glomerulonephritis, vasculitis, peripheral neuropathy, arthritis, and/or pulmonary symptoms of hemoptysis and dyspnea. (See Dammacco et al., supra; Sansonno et al., supra; Ferri et al., Cleve. Clin. J. Med. 69 Suppl 2:SII20-23, 2002 (“Ferri et al. I”); Ferri et al., J. Clin. Pathol. 55:4-13, 2002 (“Ferri et al. II”).) Cryoglobulinemia can be observed in association of a variety of disorders including multiple myeloma, lymphoproliferative disorders, connective tissue diseases, infection, and liver disease. (Ferri et al. I, supra; Ferri et al. II, supra.) Before the discovery of hepatitis C virus (HCV) and prior to development of methods to detect anti-HCV antibodies, patients without identifiable underlying disease were considered to have idiopathic or “essential” mixed cryoglobulinemia. It is now known that “essential” mixed cryoglobulinemia is strongly associated with HCV infection and encompasses the majority of patients with types II and III cryoglobulinemia. (See Sansonno et al., supra.) Current evidence suggests that essential mixed cryoglobulinemia occurs when an aberrant immune response to hepatitis C infection leads to the formation of immune complexes consisting of hepatitis C antigens, polyclonal hepatitis C-specific IgG, and monoclonal IgM rheumatoid factor. The deposition of these immune complexes within susceptible tissue sites triggers an inflammatory cascade that results in the clinical syndrome of essential mixed cryoglobulinemia. (Dammacco et al., supra; Sansonno et al., supra.)

Cryoglobulinemia is also associated with a variety of other infections in addition to HCV (see Ferri et al. II, supra), including those of viral origin such as cytomegalovirus (CMV), Epstein-Barr virus (EBV), human immunodeficiency virus (HIV-1), and hepatitis B virus (HBV), those of bacterial origin including Mycoplasma pneuymoniae, Treponema pallidum (syphilis), Mycobacterium tuberculosis, Coxiella Burnetti Q fever, Brucella, and infections with parasites such as Toxoplasma gondii and Visceral leishmaniasis.

Essential mixed cryoglobulinemia is considered to be a primary vasculitis disorder. The Chapel Hill Consensus Conference (CHCC) classification of vasculitis is based on the size of the affected vessels and groups the diseases into those affecting large-, medium-, or small-vessels. (See Jennette et al., Cleve. Clin. J. Med. 69 Suppl 2:SII33-38, 2002; Fiorentino, J. Am. Acad. Dermatol. 48:311-340, 2003.) Importantly, two vasculitis syndromes are associated with deposition of immune complexes: Henoch-Schonlein purpura is associated with deposition of IgA-containing immune complexes; and essential cryoglobulinemic vasculitis is associated with deposition of IgG/IgM immune complexes. (See Fiorentino, supra.)

The incidence of HCV infection in essential mixed cryoglobulinemia ranges from 40-100% in reported cases, depending on geography. Approximately 200 million worldwide are chronically infected with HCV, with 3.5 million new infections reported each year. (See Sy and Jamal, Int. J. Med. Sci. 3:41-46, 2006.) The USA incidence and prevalence are 30,000 new infections per year and 3.9 million with chronic infections. (See Sy and Jamal, supra.) Approximately 50-60% of patients with chronic HCV infections have cryoglobulins in their serum and overt cryoglobulinemic syndromes develop in about 5% of cases. (See Sansonno et al., supra; Sansonno and Dammacco, supra.) Hepatitis B virus has been described as an etiologic agent in 5% of patients with mixed cryoglobulinemia. (See Ferri et al. I, supra.)

The current therapies for cryoglobulinemia include low dose steroids for moderate disease and combinations of steroids, cyclophosphamide, or plasmapheresis are used for more severe forms of disease. Patients with active HCV-mediated hepatitis are often treated with a combination of interferon-α and ribavirin.

The efficacy of the FcγRIA polypeptides of the invention can be tested in vivo in animal models of disease. A particularly suitable animal model for evaluating efficacy of soluble FcγRIA against immune complex-mediated disease, including cryoglobulinemia, are mice over-expressing thymic stromal lymphopoietin (TSLP), an interleukin-7 (IL-7)-like cytokine with B-cell promoting properties. TSLP mice produce large amounts of circulating cryoglobulins of mixed IgG-IgM composition. (See Taneda et al., Am. J. Pathol 159:2355-2369, 2001.) Development of mixed cryoglobulinemia in these animals is associated with systemic inflammatory disease involving kidneys, liver, lungs, spleen, and skin (see Taneda et al., supra) due to immune complex deposition in these tissues. Kidney disease in these animals closely resembles human cryoglobulinemia glomerulonephritis as seen in patients with HCV infection. A role for Fcγ receptors in the disease process was shown by the exacerbation of renal injury with accelerated morbidity and mortality after deletion of the inhibitory receptor Fcγ receptor IIb. (See Muhlfeld et al., Am. J. Pathol 163:1127-1136, 2003.) Treatment of TSLP-transgenic mice with recombinant soluble FcγRIA in accordance with the present invention is further described in Example 18, infra.

2. Systemic Lupus Erythematosus

Systemic lupus erythematosus (SLE) is a complex, multi-organ (systemic) autoimmune disorder characterized by the production of pathogenic autoantibodies with subsequent deposition of immune complexes, which results in widespread tissue damage. Although the etiology of SLE is unknown, multiple genetic, environmental, and hormonal factors are thought to play a role in disease. (See Hahn, “Systemic Lupus Erythematosus” in Harrison's Principles of Internal Medicine (Kasper et al. eds., McGraw-Hill, New York 2005).) SLE is clinically characterized by a waxing and waning course and by involvement of multiple organs including skin, kidneys, and central nervous system (Lupus: Molecular and Cellular Pathogenesis (Kammer and Tsokos eds., Human Press, N.J., 1st ed. 1999); Systemic Lupus Erythromatosus (Lahita ed., Academic Press, Amsterdam, 3rd ed. 1999)). Thus, the disease displays a broad variety of symptoms and clinical features, including systemic, cutaneous, renal, musculoskeletal, and hematologic.

The overall prevalence of SLE is about one in 2000, and about one in 700 Caucasian women develops SLE during her life time. (Lahita, Curr. Opin. Rheumatol. 11:352-6, 1999). In the United States alone, over half a million people have SLE, and most are women in their childbearing years (Hardin, J. Exp. Med. 185:1101-1111, 2003).

There is no single criteria to diagnose SLE. The American College of Rheumatology has developed 11 criteria to diagnose SLE, which span the clinical spectrum of SLE in aspects of skin, systemic, and laboratory tests. These criteria include malar rash, discoid rash, sensitivity to sun light, oral ulcers, arthritis, serositis, kidney and central nervous system inflammation, blood alterations, and the presence of antinuclear antibodies. A patient must meet four of these criteria in order to be classified as a SLE patient. (Tan et al., Arthritis Rheumatol. 25:1271-1277, 1982). SLE is usually confirmed by tests including, but not limited to, blood tests to detect anti-nuclear antibodies; blood and urine tests to assess kidney function; complement tests to detect the presence of low levels of complement that are often associated with SLE; a sedimentation rate (ESR) or C-reactive protein (CRP) to measure inflammation levels; X-rays to assess lung damage and EKGs to assess heart damage.

The standard therapy for SLE is administration of the steroid glucocorticoid, a general immune response inhibitor. It can be used to relieve symptoms; however, no cure for SLE is currently available. Low dose p.o. prednisone at a level less than 0.5 mg/kg/day is usually given. Unfortunately, this therapy is insufficient to keep patients in remission, and flaring of the disease is frequent. Flares can be controlled with high dose glucocorticoid via intravenous pulses at 30 mg methylprednisolone/kg/day for 3 consecutive days. However, steroid treatment at high dosage can present severe side effects for patients.

These standard treatments are generally nonspecific, are frequently associated with serious side-effects and do not significantly affect the progression of the disease or transition to life threatening kidney complications (lupus nephritis or LN). Consequently, there is a long-felt need in the art to develop new methods for treating SLE.

3. Rheumatoid Arthritis

Rheumatoid arthritis (RA) is characterized by chronic joint inflammation that typically leads to tissue damage and joint deformation. Although the precise etiology is not clear, it is generally thought to be an autoimmune disease with roles played by immune complexes, a variety of lymphoid cell types (T-cells, B-cells, neutrophils, macrophages, a number of pro-inflammatory cytokines such as TNF-α and IL-1β. (See Harrison's Principles of Internal Medicine (Kasper et al. eds., McGraw-Hill, New York 2005); Olsen and Stein, N. Engl. J. Med. 350:2167-2179, 2004.)

Rheumatoid arthritis is a systemic disease that affects the entire body and is one of the most common forms of arthritis. RA is immune-mediated and is particularly characterized by inflammation and subsequent tissue damage leading to severe disability and increased mortality. In particular, it is characterized by the inflammation of the membrane lining the joint, which causes pain, stiffness, warmth, redness and swelling. Inflammatory cells release enzymes that may digest bone and cartilage. As a result of rheumatoid arthritis, the inflamed joint lining, the synovium, can invade and damage bone and cartilage leading to joint deterioration and severe pain amongst other physiologic effects. The involved joint can lose its shape and alignment, resulting in pain and loss of movement.

A variety of cytokines are produced locally in the rheumatoid joints. Numerous studies have demonstrated that IL-1 and TNF-α, two prototypic pro-inflammatory cytokines, play an important role in the mechanisms involved in synovial inflammation and in progressive joint destruction. Indeed, the administration of TNF-α and IL-1 inhibitors in patients with RA has led to a dramatic improvement of clinical and biological signs of inflammation and a reduction of radiological signs of bone erosion and cartilage destruction. However, despite these encouraging results, a significant percentage of patients do not respond to these agents, suggesting that other mediators are also involved in the pathophysiology of arthritis (Gabay, Expert. Opin. Biol. Ther. 2:135-149, 2002). Since RA is characterized by the presence of antibodies directed against Type II collagen, a major extracellular matrix component of joint cartilage, these antibodies are thought to mediate the release of the inflammatory cytokines, such as those described above, through their interaction with synoviocytes or other inflammatory cell types within the joint space.

Immunologic abnormalities that may be important in the pathogenesis of RA also include immune complexes found in joint fluid cells and in vasculitis. Contributing to these complexes are antibodies (such as RF) produced by plasma cells and T helper cells that infiltrate the synovial tissue and which can produce pro-inflammatory cytokines. Macrophages and their cytokines (e.g., TNF, GMCS-F) are also abundant in diseased synovium. Increased levels of adhesion molecules contribute to inflammatory cell emigration and retention in the synovial tissue. Increased macrophage-derived lining cells are also prominent, along with some lymphocytes.

Established treatments of RA include disease modifying anti-rheumatic drugs (DMARD) such as hydroxychloroquine, sulfasalazine, methotrexate, leflunomide, rituximab, infliximab, azathioprine, D-penicillamine, Gold (oral or intramuscular), minocycline and cyclosporine, coritcosteroids such as prednisone and non-steroidal anti-inflammatory drugs (NSAIDS). These treatments are generally nonspecific, are frequently associated with serious side-effects and do not significantly affect the progression of joint destruction. Consequently, there is a long-felt need in the art to develop new methods for treating RA.

The soluble FcγRIA polypeptides of the present invention could block the interaction of the immune complexes with inflammatory cell types in the synovium and prevent inflammation. Therefore, the FcγRIA polypeptides of the invention could serve as a valuable therapeutic to reduce inflammation in rheumatoid arthritis, and other arthritic diseases.

There are several animal models for rheumatoid arthritis known in the art. For example, in the collagen-induced arthritis (CIA) model, mice develop chronic inflammatory arthritis that closely resembles human rheumatoid arthritis. Since CIA shares similar immunological and pathological features with RA, this makes it an ideal model for screening potential human anti-inflammatory compounds. The CIA model is a well-known model in mice that depends on both an immune response, and an inflammatory response, in order to occur. The immune response comprises the interaction of B-cells and CD4+ T-cells in response to collagen, which is given as antigen, and leads to the production of anti-collagen antibodies. The inflammatory phase is the result of tissue responses from mediators of inflammation, as a consequence of some of these antibodies cross-reacting to the mouse's native collagen and activating cellular Fc receptors and/or the complement cascade. An advantage in using the CIA model is that the basic mechanisms of pathogenesis are known. The relevant T-cell and B-cell epitopes on type II collagen have been identified, and various immunological (e.g., delayed-type hypersensitivity and anti-collagen antibody) and inflammatory (e.g., cytokines, chemokines, and matrix-degrading enzymes) parameters relating to immune-mediated arthritis have been determined, and can thus be used to assess test compound efficacy in the CIA model (Wooley, Curr. Opin. Rheum. 3:407-20, 1999; Williams et al., Immunol. 89:9784-788, 1992; Myers et al., Life Sci. 61:1861-78, 1997; and Wang et al., Immunol. 92:8955-959, 1995).

The administration of FcγRIA polypeptides of the present invention to these CIA model mice can be used to evaluate the use of soluble FcγRIA to ameliorate symptoms and alter the course of disease. By way of example and without limitation, the injection of 0.1 mg to 2.0 mg of an FcγRIA polypeptide of the invention, such as a soluble FcγRIA (e.g., a soluble polypeptide comprising residues 16-282 or 16-292 of SEQ ID NO:2) per mouse (one to seven times a week for up to but not limited to 4 weeks via s.c., i.p., or i.m route of administration) can significantly reduce the disease score (paw score, incident of inflammation, or disease). Depending on the initiation of administration (e.g., prior to or at the time of collagen immunization, or at any time point following the second collagen immunization, including those time points at which the disease has already progressed), antagonists of the present invention can be efficacious in preventing rheumatoid arthritis, as well as preventing its progression. For example, as shown by studies described herein, administration of a soluble FcγRIA polypeptide (residues 16-282 of SEQ ID NO:2) ameliorated symptoms and altered the course of disease in the mouse CIA model. (See Example 19, infra.)

Another model for immune complex mediated rheumatic disease is the collagen antibody-induced model of arthritis in mice. (See Terato et al., J. Immunol. 48: 2103-2108, 1992.) Joint disease is induced in this model by the intravenous injection of a cocktail of four monoclonal antibodies, such as Arthrogen-CIA® from Chemicon, directed against Type II collagen. Arthrogen-CIA® used for the induction of arthritis in mice is a mixture of four clones that recognize individual epitopes within an 83 amino acid peptide within the CB11 domain of type II collagen (Chemicon International technical brochure). These epitopes are similar in type II collagen from human, mice, cow, chicken, monkey, and rat. The antibodies localize to the joints of mice, where they form immune complexes with cartilage-specific type II collagen. The antigen-antibody immune complexes are thought to induce disease through their interaction with Fc gamma receptors located on the surface of inflammatory cell types within the joint. Typically, on day 0, 2-4 mg of Arthrogen-CIA cocktail is injected into mice by intravenous dosing. This is followed three days later with an intraperitoneal injection of 50-100 μg of LPS. (See Terato et al., Autoimmunity 22:137-147, 1995.) Arthritis, evident as red and swollen paws, develops with 1-2 days. In a typical experiment, the mice are treated on day 0 or day 3 by injection with soluble FcγRIA (100-2000 μg protein) dissolved in a suitable vehicle. Dosing with soluble FcγRIA can, for instance, be given every other day starting on day 0 or day 3. The arthritis score for each animal can be assessed everyday joint swelling and joint thickness. In a typical experiment, soluble FcγRIA decreases the arthritis score.

4. Mixed Connective Tissue Disease

Mixed connective tissue disease is a rare disorder characterized by clinical features of SLE, systemic sclerosis, polymyositis or dermatomyositis, and RA and by very high titers of circulating antinuclear antibody to a ribonucleoprotein (RNP) antigen. (See Harrison's Principles of Internal Medicine, supra; Kim and Grossman, Rheum. Dis. Clin. North Am. 31:549-565, 2005; Venables, Lupus 15:132-137, 2006.) This antibody in high titer, now referred to as anti-U1 RNP, has been a justification for considering MCTD as a distinct clinical entity. MCTD has been challenged as a distinct disorder by those who consider it as a subset of SLE or scleroderma. Others prefer to classify MCTD as an undifferentiated connective tissue disease. Hand swelling, Raynaud's phenomenon, polyarthralgia, inflammatory myopathy, esophageal hypomotility, and pulmonary dysfunction are common. Diagnosis is by the combination of clinical features, antibodies to RNP, and absence of antibodies specific for other autoimmune diseases. In some patients, the disorder evolves into classic systemic sclerosis or SLE.

Raynaud's phenomenon may precede other manifestations by years. Frequently, the first manifestations resemble early SLE, scleroderma, polymyositis or dermatomyositis, or RA. Whatever the initial presentation, limited disease tends to progress and become widespread, and the clinical pattern changes over time. The most frequent finding is swelling of the hands that eventually produces a sausagelike appearance of the fingers. Skin findings include lupus or dermatomyositis-like rashes. Diffuse scleroderma-like skin changes and ischemic necrosis or ulceration of the fingertips are much less frequent in MCTD. Almost all patients have polyarthralgias, and 75% have frank arthritis. Often the arthritis is non-deforming, but erosive changes and deformities similar to those in RA may be present. Proximal muscle weakness with or without tenderness is common. Renal disease occurs in about 10% and is often mild but occasionally causes morbidity or mortality. A trigeminal sensory neuropathy develops more frequently in MCTD than in other connective tissue diseases. Rheumatoid factors are frequently positive, and titers are often high. The ESR is frequently elevated.

MCTD is typically suspected when additional overlapping features are present in patients appearing to have SLE, scleroderma, polymyositis, or RA. Patients are first tested for antinuclear antibodies (ANA) and antibody to extractable nuclear antigen (ENA) and RNP antigen. If results of these tests are compatible with MCTD (e.g., RNP antibodies very high), γ-globulin level, serum complement levels, rheumatoid factors, anti Jo-1 (anti histidyl t-RNA synthetase), and antibodies to the ribonuclease-resistant Smith (Sm) component of ENA, and double-stranded DNA are tested to exclude other possible diagnoses. Further workup depends on symptoms and signs; manifestations of myositis, renal involvement, or pulmonary involvement prompt tests of those organs (e.g., CPK, MRI, electromyogram, or muscle biopsy for diagnosis of myositis).

The overall 10-yr survival rate is 80%, but prognosis depends largely on which manifestations predominate. Causes of death include pulmonary hypertension, renal failure, MI, colonic perforation, disseminated infection, and cerebral hemorrhage. Some patients have sustained remissions for many years without treatment.

Mixed connective tissue disease (MCTD) occurs worldwide and in all races, with a peak incidence in the teens and 20s but MCTD is seen in children and the elderly. Women are predominantly affected. The incidence and prevalence has not been clearly established. In most studies, the number of patients with clinical and serologic features of MCTD is ˜4-fold fewer than for SLE, suggesting an overall prevalence of about 10/100,000. (See Harrison's Principles of Internal Medicine, supra; Venables, supra.)

Current treatment for MCTD is similar to that for SLE, with corticosteroids if disease is moderate or severe. Most patients with moderate or severe disease respond to corticosteroids, particularly if treated early. Mild disease is often controlled by salicylates, other NSAIDs, anti-malarials, or sometimes low-dose corticosteroids. Severe major organ involvement usually requires higher doses of corticosteroids.

5. Polyarteritis Nodosa-HBV Associated

Originally described by Kussmaul and Maier in 1866, classic polyarteritis nodosa (PAN) is a multisystem disorder characterized by a wide range of symptoms. (See Fiorentino, J. Am. Acad. Dermatol. 48:311-340, 2003; Harrison's Principles of Internal Medicine, supra). PAN is a necrotizing vasculitis of small and medium-sized muscular arteries with characteristic involvement of renal and visceral arteries. The lesions are segmental and tend to involve bifurcations and branchings of arteries. In the acute stages of the disease, neutrophils infiltrate all layers of the vessel wall and perivascular areas, resulting in intimal proliferation and degeneration of the vessel wall. As the lesion progresses, mononuclear cells infiltrate the area, resulting in fibrinoid necrosis of the vessels with compromise of the lumen, thrombosis, infarction of the tissues supplied by the vessels, and hemorrhage. (See Fiorentino, supra.)

The presence of hepatitis B antigenemia is 10-30% of patients with systemic vasculitis, particularly of the PAN type, together with the isolation of circulating immune complexes composed of hepatitis B viral antigens, suggest an immunologic role in pathogenesis of the disease. This notion is supported by findings of deposition of hepatitis B antigen, IgM, and complement in blood vessel walls of patients with this disease. (See Fiorentino, supra.)

Patients usually present with fever, weight loss, arthralgias, and malaise. Muscle wasting, abdominal pain, mononeutitis complex, hypertension, orchitis, and congestive heart failure are major symptoms demonstrating vascular involvement of the respective organ systems. If secondary to hepatitis B infection, the clinical findings are the same. The prognosis of untreated PAN is poor, with a reported 5-year survival rate of 10-20%. (See Harrison's Principles of Internal Medicine, supra.) Death usually results from gastrointestinal complications, particularly bowel infarcts and perforaton and by cardiovascular causes.

It is difficult to establish an accurate incidence of PAN because previous reports have combined the incidence of PAN with microscopic polyangiitis and related vasculitis disorders. The incidence of PAN has been estimated, however, at 5-9 cases per million (see Fiorentino, supra) and it is estimated that ˜6% of cases are due to HBV infection although a range of frequency from 10-54% has been reported.

PAN patients are currently treated with steroids with or without cyclophosphamide. (See Fiorentino, supra.) For patients with HBV, antiviral treatment with interferon-α with or without vidarabine and lamivudine is effective when combined with plasma exchange. (See Fiorentino, supra; Harrison's Principles of Internal Medicine, supra.)

6. Pemphigus Vulgaris

Pemphigus vulgaris (PV) is a blistering skin disease observed most commonly in elderly patients. The disease is characterized by the loss of cohesion between epidermal cells of the skin with the resulting formation of intraepidermal blisters. Direct immunofluorescence analysis of lesional or intact patient skin shows deposits of IgG on the surface of keratinocytes. Such deposits are derived from circulating IgG autoantibodies against desmogleins, transmembrane glycoproteins of the Ca²⁺ dependent cadherin family. PV can be life threatening. The current mainstay of treatment is systemic steroids, such as prednisone. Other immunosuppressants such as azathioprine or mycophenolate mofetil are also used. (See Harrison's Principles of Internal Medicine, supra.)

7. Diseases Associated with Exogenous Antigens

Exogenous antigens produce a wide variety of immune complex diseases including those caused by infection with viruses, bacteria, or parasites as well as serum sickness caused by exposure to foreign proteins or drugs. (See Jancar and Crespo, supra; Harrison's Principles of Internal Medicine, supra; Knowles and Shear, Dermatol. Clin. 25:245-253, 2007; Wolf et al., Clin. Dermatol. 23:171-181, 2005.) The bacterial infections associated with tissue immune complex deposition include: streptococcal, staphylococcal and meningococcal; bartonellosis, borreliosis, leprosy, syphilis, and leptospirosis. The viral infections include: Hepatitis B (polyarteritis nodosa), Hepatitis C (cryoglobulinemia), HIV-related immune complex nephropathy, human parvovirus B19 infection, CMV infection, infectious mononucleosis, and dengue hemorrhagic fever. The parasitic diseases include: Trypansoma, Plasmodium, Toxoplasma, and Schistosoma.

Currently, the most common serum sickness-like reactions are due to exposure to non-protein drugs. Drugs that have been implicated in serum-sickness-like reactions include: allopurinal, arsenicals and mercurial derivatives, barbiturates, bupropion, cephalosporins, furazolidone, gold salts, griseofulvin, hydralazine, infliximab, iodides, methyldopa, penicillins, phenyloin, piperazine, procainamide, streptokinase, and sulfonamides. Other causes of serum sickness like reactions include exposure to heterologous serum, allergen extracts, blood products, hormones, hymenoptera venom, and vaccines.

B. Other Diseases Involving Antibody Production

1. Idiopathic Thrombocytopenia Purpura (ITP)

Idiopathic thrombocytopenia purpura (ITP) is a systemic autoimmune illness characterized by the presence of autoantibodies (IgG>IgM) directed against specific platelet membrane glycoproteins that results in platelet destruction (leading to thrombocytopenia), and which is characterized by extensive ecchymoses and hemorrhages from mucous membranes, anemia, and extreme weakness. (See Harrison's Principles of Internal Medicine, supra; Cines and McMillan, Annu. Rev. Med. 56:425-442, 2005; Stasi and Provan, Mayo Clin. Proc. 79:504-522, 2004.)

The platelet count becomes exceedingly low and spontaneous bleeding from the gums, gastrointestinal tract and nose can be seen. Purpura refers to the purplish-looking areas of the skin and mucous membranes (such as the lining of the mouth) where bleeding has occurred as a result of decreased platelets. Physical examination may demonstrate enlargement of the spleen. A typical rash occurs due to microscopic hemorrhage of small blood vessels in the skin. Platelet counts under 10,000 can lead to spontaneous hemorrhage into the brain, causing death. Also called immune thrombocytopenic purpura, purpura hemorrhagica, thrombocytopenic purpura, Werlhof's disease. Although most cases are asymptomatic, very low platelet counts can lead to a bleeding diathesis and purpura. There are two types of ITP, acute ITP that affects children (similar incidence in males and females) and chronic ITP affecting adults (more often women; 2.6 to 1; 72% of ITP patients older than 10 are women). Most children recover without treatment. Peak prevalence in children is 2-4 years, and in adults is 20-50 years; approximately 40% of all ITP patients are younger than 10 years old.

Incidence of ITP: 4-8 per 100,000 children per year, 66 cases per million adults, 50 cases per million children. New cases of chronic refractory ITP comprise ˜10 cases per million per year. The number of individuals in the United States with ITP has been estimated to be approximately 200,000. There are about 100 total new cases of ITP per million people per year. Approximately half of the new cases are in children.

Mild ITP does not require treatment. When platelet counts fall under 10,000 per microliter, or under 50,000 when hemorrhage occurs (e.g., in the digestive tract or in a severe nosebleed) treatment is generally initiated with steroids. (See Cines and McMillan, supra.) Intravenous immunoglobulin (IVIg) is used for life threatening cases. Later, so-called steroid-sparing agents (alternatively called DMARDs) may be used. When these strategies fail, splenectomy is often undertaken, as platelets targeted for destruction will often meet their fate in the spleen. A relatively new strategy is treatment with anti-D, an agent usually used in mothers who have been sensitized to rhesus antigen by an Rh+ baby. Other chemotherapeutic drugs such as vincristine, azathioprine (Imuran), Danazol, cyclophosphamide, and cyclosporine are prescribed for patients only in the severe case where other treatments have not shown benefit since these drugs have potentially harmful side effects. IVIg, while effective, is expensive and the improvement is temporary (generally lasting less than a month). However, in the case of a pre-splenectomy ITP patient with dangerously low platelet counts, and a poor response to other treatments, IVIg treatment can increase platelet counts, making the splenectomy operation less dangerous.

2. Sjogren's Syndrome

Sjogren's syndrome (SS) is a chronic autoimmune disorder characterized by lymphocytic infiltration of salivary and lacrimal glands, resulting in dry eyes and dry mouth. It is classified as either primary (autoimmune sicca (dryness) syndrome without underlying connective tissue disorder) or secondary (autoimmune-mediated sicca syndrome in a patient with ongoing connective tissue disorder like RA, SLE or SSc). (See Harrison's Principles of Internal Medicine, supra.) The female-to-male ratio for SS is 9:1, with a mean age at diagnosis of 60 years. A model of pathogenesis postulates a virus or environmental insult in the appropriate genetic/hormonal background leads to epitheliitis in the salivary and lacrimal glands. The resulting mononuclear cell infiltrates (˜70% CD4+ T-cells, 25% CD8+ T-cells, 20-30% B-cells) release cytokines (IFNγ), which in turn activate macrophages that release proinflammatory cytokines: TNFα, IL-1β and IL-6. These cytokines then cause the release of MMPs from acinar cells, which degrade the basement membrane collagen. In time, the glandular tissue is replaced with scar tissue and fat. (See Harrison's Principles of Internal Medicine, supra.)

In addition to dry mouth/eye symptoms, other symptoms can include: esophageal dysmotility, peripheral neuropathy arthralgia and fibromyalgia. 60% of patients present with autoantibodies (rheumatoid factor, ANA, Ro/SS-A, La/SS-B) and suffer extreme fatigue. SS patients are reported to have 44 times higher risk for developing lymphoma.

A variety of treatments have been used for SS including NSAIDs, steroids, hydroxychloroquine, and methotrexate. Several anti-cytokine therapies are also in use but are not recommended as first-line therapy. These include: REMICADE, ENBREL, IFN-α, anti-IFN-γ, RITUXAN, cyclosporine, tacrolimus, and various topical ophthalmic preparations.

3. Antiphospholipid Antibody Syndrome

The antiphospholipid antibody syndrome is a common autoimmune prothrombotic condition characterized by arterial and/or venous thrombosis and pregnancy morbidity associated with persistently positive anti-cardiolipin antibodies and/or lupus anticoagulant. (See Harrison's Principles of Internal Medicine, supra; Blume and Miller, Cutis 78:409-415, 2006; Fischer et al., Semin. Nephrol. 27:35-46, 2007.) Recent evidence that some of these antibodies (IgG and IgM) are directly against phospholipid binding proteins (B2-glycoprotein 1, prothrombin, protein C, protein S, TPA, and annexin V rather than the negatively charged phospholipids themselves). APS can occur in association with other autoimmune disease, most commonly with SLE (secondary APS) or as an isolated disorder (primary APS).

APS affects any size of vessel and any organ of the body. Clinical features include peripheral venous and arterial thrombosis (deep vein thrombosis), fetal loss, skin disease, cardiac and pulmonary manifestations, renal involvement, and neurological disorders (stroke). Thrombotic complications are the main cause of death in SLE patients.

APS is a common cause of acquired thrombophilia, with an estimated 35,000 new cases of APS-associated venous thrombosis and 5000 new cases of arterial thrombosis in the U.S. per year. Patients with APS antibodies are 3-10 times more likely to have a recurrent thrombosis than patients without these antibodies. In the U.S., about 2% of the general population tests positive for anti-phospholipid antibodies (AAs), including lupus anticoagulant, anti-cardiolipin antibodies or both. AAs were detected in 46% of patients with stroke or transient ischemic attack under 50 years of age and in 21% of young survivors (<45 years of age) of myocardial infarction. The prevalence of AAs in patients with SLE is very high (30-50%). The prevalence of elevated AAs in dialysis patients varies between 0.7% and 69% in the published literature. In patients with APS, the ratio of women to men is about 2 to 1 for the primary form and 9 to 1 for cases associated with SLE.

The current therapy for patients who have APS but haven't experienced thrombotic events or cutaneous changes is lifelong therapy with low dose aspirin. A patient with medium to high AA titers or thrombosis needs immediate treatment with an anticoagulant such as heparin. Long-term therapy is anticoagulation with warfarin. There is some clinical trial activity of using cyclophosphamide in patients with life-threatening APS and using steroids to control APS-associated pregnancy loss. There is not much precedent for using anti-B cell therapies for controlling the levels of AAs.

4. Dermatomyositis

Dermatomyositis is a progressive condition characterized by symmetric proximal muscular weakness with elevated muscle enzyme levels and a skin rash, typically a purplish-red on the face, and edema of the eyelids and periorbital tissue. (See Dalakas, Curr. Opin. Pharmacol. 1:300-306, 2001; Dalakas, Nat. Clin. Pract. Rheumatol. 2:219-227, 2006.) Affected muscle tissue shows degeneration of fibers with a chronic inflammatory reaction, occurs in children and adults, and in the latter may be associated with visceral cancer. The cause of PM/DM is unknown. Although it rarely occurs in multiple family members, it may be linked to certain HLA types (e.g., DR3, DR5, or DR7). Infectious agents, including viruses, and Toxoplasma and Borrelia species, have been suggested as possible triggers of the disease. Several cases of drug-induced disease have been reported (e.g. hydroxyurea, penicillamine, statins, quinidine, and phenylbutazone). Immunological and humoral abnormalities are common (e.g., increased TNF-α in muscle, circulating myositis-specific autoAbs; abnormal T- and B-cell activity; family history of other autoimmune diseases). B cells are the most abundant inflammatory cells at the perivascular sites.

Dermatomyositis is associated with skin problems (typically a purplish-red rash on the face, and edema of the eyelids and periorbital tissue) and since articular, cardiac, pulmonary, and gastrointestinal manifestations occur in up to 50% of the patients, the illness can be associated with severe morbidity. It is often associated with other connective tissue autoimmune diseases, such as SLE, scleroderma, and RA. Unlike RA, arthritis associated specifically with DM/PM is not erosive or deforming. Consistent with skin changes associated with other autoimmune connective tissue diseases, such as SLE, there are perivascular inflammatory infiltrates in the skin. PM/DM is not usually life-threatening, but patients often develop residual weakness, disability, and reduced Quality of Life. PM/DM may cause death because of severe muscle weakness and/or cardiopulmonary involvement. Risk of malignancy is very high in patients with DM (incidence ratio=26) but not PM; the malignancy occurs more frequently in adults older than 60 years. Calcinosis (manifested by firm, yellow- or flesh-colored nodules) of the skin or muscle is unusual in adults but occurs in up to 40% of children or adolescents with DM; it is very debilitating. They can extrude through the surface of the skin, in which case secondary infection may occur.

The incidence of inflammatory myopathies (polymyositis alone, and polymyositis and dermatomyositis combined has been estimated at 0.1 and 1 per 100,000 people, respectively (no ethnic bias), and is apparently increasing. Prevalence is 1 and 6 per 100,000 for PM alone and PM/DM combined, respectively. Females are affected more than males (˜2:1). PM/DM can occur in people of any age. Two peak ages of onset exist. In adults, the peak age of onset is approximately 50 years, and, in children, the peak age is approximately 5-10 years.

The mainstay of treatment is steroids. (See Dalakas, Jama 291:2367-2375, 2004; Dalakas, Pharmacol. Ther. 102:177-193, 2004.) Immunosuppressant therapy with methotrexate, azathioprine, and mycophenolate mofetil have also been used. In refractory patients, IVIg has been used for short-term therapy. Emerging therapies for this disorder include Rituxan. Although there is some concern that TNF antagonists may increase some of the risks associated with DM (infection, malignancy, induction of other autoimmune disease), Remicade and Enbrel are being studied in ongoing clinical trials for this disorder.

5. Guillain-Barre Syndrome

Guillian-Barre syndrome is a severe post infectious neurological disorder. The nerve damage observed in GBS patients is presumably caused by cross-reactive anti-ganglioside antibodies. The cellular immunological background of the production of cross-reactive antibodies in GBS is largely unknown. Some have hypothesized that a differential response of dendritic cells to the most frequent antecedent infection in GBS, Campylobacter jejuni, results in enhanced B-cell proliferation and differentiation into autoreactive plasma cells. Host related factors as well as pathogenic factors may be related to this. (See Harrison's Principles of Internal Medicine, supra; Lewis, Neurol. Clin. 25:71-87, 2007; Said, Neurol. Clin. 25:115-137, 2007; Yuki, Muscle Nerve 35:691-711, 2007.)

GBS is a devastating disorder with a mortality of 5-15%. IVIg are the first choice treatment for these patients. (See Harrison's Principles of Internal Medicine, supra). Still, about 50% of patients are unable to walk independently after 6 months. GBS consists of at least four subtypes of acute peripheral neuropathy. The histological appearance of the acute inflammatory demyelinating polyradiculoneuropathy (AIDP) subtype resembles experimental autoimmune neuritis, which is predominantly caused by T cells directed against peptides from the myelin proteins P0, P2, and PMP22. The role of T-cell-mediated immunity in AIDP remains unclear and there is evidence for the involvement of antibodies and complement. Strong evidence now exists that axonal subtypes of GBS, acute motor axonal neuropathy (AMAN), and acute motor and sensory axonal neuropathy (AMSAN), are caused by antibodies to gangliosides on the axolemma that target macrophages to invade the axon at the node of Ranvier. About a quarter of patients with GBS have had a recent Campylobacter jejuni infection, and axonal forms of the disease are especially common in these people. The lipo-oligosaccharide from the C. jejuni bacterial wall contains ganglioside-like structures and its injection into rabbits induces a neuropathy that resembles acute motor axonal neuropathy. Antibodies to GM1, GM1b, GD1a, and GalNac-GD1a are in particular implicated in acute motor axonal neuropathy and, with the exception of GalNacGD1a, in acute motor and sensory axonal neuropathy. The Fisher's syndrome subtype is especially associated with antibodies to GQ1b, and similar cross-reactivity with ganglioside structures in the wall of C. jejuni has been discovered. Anti-GQ1b antibodies have been shown to damage the motor nerve terminal in vitro by a complement-mediated mechanism.

GBS is a rare disorder and affects men and women equally in the US (NIH, The National Women's Health Centre, 2004). GBS affects 1 person per 100,000 population in the US (NIH, The National Women's Health Centre, 2004). The U.S. prevalence of all chronic inflammatory demyelinating polyneuropathies (CIDP), including GBS is about ˜1 to 7.7 per 100,000 (2,000-15,000 cases in U.S.). However, this is probably an underestimate, assuming that CIDP constitute 5% of all neuropathies (10 million cases), then one might expect there are actually ˜300,000 (active)-500,000 cases in total.

IVIg and plasmapheresis are currently used as therapy for GBS. Since GBS is an autoimmune neuropathy, it is anticipated that therapies directed towards T-cells, B-cells, and/or complement may be useful in these diseases.

6. Goodpasture's Syndrome

The term “Goodpasture's syndrome” (GPS) is an eponym derived from a report in 1919 by Ernest Goodpasture, who described the clinical syndrome of pulmonary hemorrhage associated with influenza infection and the histologic finding of acute crescentic glomerulonephritis. Over the years, the terminology has been used in different ways by different persons, some including all causes of pulmonary hemorrhage with renal dysfunction as GPS. Others limited the term GPS to patients with pulmonary hemorrhage associated with anti-glomerular basement membrane (anti-GBM) antibodies, as opposed to glomerulonephritis with anti-GBM antibodies but without pulmonary hemorrhage. Yet others espouse the concept of anti-type-IV collagen disease rather than GPS. It seems likely that, as we gain a better understanding of the pathogenesis of this disease, we will be able to define why the clinical spectrum is so variable and will be able to develop more rational and specific forms of therapy for GPS.

The sine qua non for the diagnosis of GPS is demonstration of bound anti-GBM antibodies in the glomeruli of the kidneys. Circulating anti-GBM antibodies are present in more than 90% of patients with anti-GBM disease. The clinical course of untreated, and even treated, GPS is bleak; this disease is associated with an extremely poor prognosis.

GPS is a rare disease, having an incidence of about 0.1 case per million people. The disease is more common in whites than in African Americans and may be more common in certain other racial groups, such as the Maoris in New Zealand. GPS can present year round, but its incidence appears to increase in the spring and early summer.

The current therapies for GPS include steroids, immunosuppressants, and plasma exchange. Since the renal pathology appears to be due to the accumulation of anti-GBM antibodies in kidney glomeruli, B-cell directed therapies may be useful in this disease.

7. Inflammatory Bowel Disease (IBD)

In the United States approximately 500,000 people suffer from Inflammatory Bowel Disease (IBD) which can affect either colon and rectum (Ulcerative colitis) or both, small and large intestine (Crohn's Disease). In both Crohn's disease and ulcerative colitis, the tissue damage results from an inappropriate or exaggerated immune response to antigens of the gut microflora. This review summarizes current knowledge regarding the role of immune-inflammatory mediators in the pathogenesis of inflammatory bowel disease. Despite having a common basis in overresponsiveness to luminal antigens, Crohn's disease and ulcerative colitis are immunologically distinct entities. Crohn's disease is associated with a Th1 T cell-mediated response, characterized by enhanced production of interferon-γ and tumor necrosis factor-α. Interleukin (IL)-12 and possibly other cytokines, govern the Th1 cell differentiation, but optimal induction and stabilization of polarized Th1 cells would require additional cytokines, such as IL-15, IL-18 and IL-21. In ulcerative colitis, the local immune response is less polarized, but it is characterized by CD 1-reactive natural killer T cell production of IL-13. Beyond these differences, Crohn's disease and ulcerative colitis share important end-stage effector pathways of intestinal injury, which are mediated by an active cross-talk between immune and non-immune mucosal cells.

Moreover, neutralization of IgG can reduce disease symptoms and pathology in animals models of IBD Nielson et al., Scand. J. Gastroenterol. 38:180, 2003; Schmidt et al., Inflamm. Bowel Dis. 11:16, 2005; Fuss et al., Inflamm. Bowel Dis. 12:9, 2006; Yen et al., J. Clin. Invest. 116:1310, 2006; Zhang et al., Inflamm. Bowel Dis. 12:382, 2006). Thus, the FcγRIA polypeptides of the present invention could serve as a valuable therapeutic to reduce inflammation and pathological effects in IBD and related diseases.

Ulcerative colitis (UC) is an inflammatory disease of the large intestine, commonly called the colon, characterized by inflammation and ulceration of the mucosa or innermost lining of the colon. This inflammation causes the colon to empty frequently, resulting in diarrhea. Symptoms include loosening of the stool and associated abdominal cramping, fever and weight loss.

Although the exact cause of UC is unknown, recent research suggests that the body's natural defenses are operating against proteins in the body which the body thinks are foreign (an “autoimmune reaction”). Perhaps because they resemble bacterial proteins in the gut, these proteins may either instigate or stimulate the inflammatory process that begins to destroy the lining of the colon. As the lining of the colon is destroyed, ulcers form releasing mucus, pus and blood. The disease usually begins in the rectal area and may eventually extend through the entire large bowel. Repeated episodes of inflammation lead to thickening of the wall of the intestine and rectum with scar tissue. Death of colon tissue or sepsis may occur with severe disease. The symptoms of ulcerative colitis vary in severity and their onset may be gradual or sudden. Attacks may be provoked by many factors, including respiratory infections or stress.

Although there is currently no cure for UC available, treatments are focused on suppressing the abnormal inflammatory process in the colon lining. Treatments including corticosteroids immunosuppressives (e.g., azathioprine, mercaptopurine, and methotrexate) and aminosalicytates are available to treat the disease. However, the long-term use of immunosuppressives such as corticosteroids and azathioprine can result in serious side effects including thinning of bones, cataracts, infection, and liver and bone marrow effects. In the patients in whom current therapies are not successful, surgery is an option. The surgery involves the removal of the entire colon and the rectum.

There are several animal models that can partially mimic chronic ulcerative colitis. One of the most widely used models is the 2,4,6-trinitrobenesulfonic acid/ethanol (TNBS) induced colitis model, which induces chronic inflammation and ulceration in the colon. When TNBS is introduced into the colon of susceptible mice via intra-rectal instillation, it induces T-cell mediated immune response in the colonic mucosa, in this case leading to a massive mucosal inflammation characterized by the dense infiltration of T-cells and macrophages throughout the entire wall of the large bowel. Moreover, this histopathologic picture is accompanied by the clinical picture of progressive weight loss (wasting), bloody diarrhea, rectal prolapse, and large bowel wall thickening (Neurath et al. Intern. Rev. Immunol. 19:51-62, 2000).

Another colitis model uses dextran sulfate sodium (DSS), which induces an acute colitis manifested by bloody diarrhea, weight loss, shortening of the colon and mucosal ulceration with neutrophil infiltration. DSS-induced colitis is characterized histologically by infiltration of inflammatory cells into the lamina propria, with lymphoid hyperplasia, focal crypt damage, and epithelial ulceration. These changes are thought to develop due to a toxic effect of DSS on the epithelium and by phagocytosis of lamina propria cells and production of TNF-alpha and IFN-gamma. Despite its common use, several issues regarding the mechanisms of DSS about the relevance to the human disease remain unresolved. DSS is regarded as a T cell-independent model because it is observed in T cell-deficient animals such as SCID mice.

The administration of the FcγRIA polypeptides of the present invention to these TNBS or DSS models can be used to evaluate the use of those polypeptides to ameliorate symptoms and alter the course of gastrointestinal disease. Moreover, the results showing inhibition of IgG or immune complex precipitation provide proof of concept that soluble FcγRIA can also be used to ameliorate symptoms in the colitis/IBD models and alter the course of disease.

8. Psoriasis

Psoriasis is a chronic skin condition that affects more than seven million Americans. Psoriasis occurs when new skin cells grow abnormally, resulting in inflamed, swollen, and scaly patches of skin where the old skin has not shed quickly enough. Plaque psoriasis, the most common form, is characterized by inflamed patches of skin (“lesions”) topped with silvery white scales. Psoriasis may be limited to a few plaques or involve moderate to extensive areas of skin, appearing most commonly on the scalp, knees, elbows and trunk. Although it is highly visible, psoriasis is not a contagious disease. The pathogenesis of the diseases involves chronic inflammation of the affected tissues. Therefore, the FcγRIA polypeptides of the present invention could serve as valuable therapeutics to reduce inflammation and pathological effects in psoriasis, other inflammatory skin diseases, skin and mucosal allergies, and related diseases.

Psoriasis is a T-cell mediated inflammatory disorder of the skin that can cause considerable discomfort. It is a disease for which there is no cure and affects people of all ages. Psoriasis affects approximately two percent of the populations of European and North America. Although individuals with mild psoriasis can often control their disease with topical agents, more than one million patients worldwide require ultraviolet or systemic immunosuppressive therapy. Unfortunately, the inconvenience and risks of ultraviolet radiation and the toxicities of many therapies limit their long-term use. Moreover, patients usually have recurrence of psoriasis, and in some cases rebound, shortly after stopping immunosuppressive therapy.

In addition to other disease models described herein, the activity of antagonists of the present invention on inflammatory tissue derived from human psoriatic lesions can be measured in vivo using a severe combined immune deficient (SCID) mouse model. Several mouse models have been developed in which human cells are implanted into immunodeficient mice (collectively referred to as xenograft models). (See, e.g., Cattan and Douglas, Leuk. Res. 18:513-22, 1994; Flavell, Hematological Oncology 14:67-82, 1996.) As an in vivo xenograft model for psoriasis, human psoriatic skin tissue is implanted into the SCID mouse model, and challenged with an appropriate antagonist. Moreover, other psoriasis animal models in ther art may be used to evaluate the present antagonists, such as human psoriatic skin grafts implanted into AGR129 mouse model, and challenged with an appropriate antagonist (see, e.g., Boyman et al., J. Exp. Med. Online publication #20031482, 2004).

Efficacy of treatment is measured and statistically evaluated as increased anti-inflammatory effect within the treated population over time using methods well known in the art. Some exemplary methods include, but are not limited to measuring for example, in a psoriasis model, epidermal thickness, the number of inflammatory cells in the upper dermis, and the grades of parakeratosis. Such methods are known in the art and described herein. (See, e.g., Zeigler et al., Lab Invest 81:1253, 2001; Zollner et al., J. Clin. Invest. 109:671, 2002; Yamanaka et al., Microbiol. Immunol. 45:507, 2001; Raychaudhuri et al., Br. J. Dermatol. 144:931, 2001; Boehncke, et al., Arch. Dermatol. Res. 291:104, 1999; Boehncke et al., J. Invest. Dermatol. 116:596, 2001; Nickoloff et al., Am. J. Pathol. 146:580, 1995; Boehncke et al., J. Cutan. Pathol. 24:1, 1997; Sugai et al., J. Dermatol. Sci. 17:85, 1998; Villadsen et al., J. Clin. Invest. 112:1571, 2003.) Inflammation may also be monitored over time using well-known methods such as flow cytometry (or PCR) to quantitate the number of inflammatory or lesional cells present in a sample, score (weight loss, diarrhea, rectal bleeding, colon length) for IBD. For example, therapeutic strategies appropriate for testing in such a model include direct treatment using the FcγRIA polypeptides of the invention.

9. Atopic Dermatitis (AD)

AD is a common chronic inflammatory disease that is characterized by hyperactivated cytokines of the helper T cell subset 2 (Th2). Although the exact etiology of AD is unknown, multiple factors have been implicated, including hyperactive Th2 immune responses, autoimmunity, infection, allergens, and genetic predisposition. Key features of the disease include xerosis (dryness of the skin), pruritus (itchiness of the skin), conjunctivitis, inflammatory skin lesions, Staphylococcus aureus infection, elevated blood eosinophilia, elevation of serum IgE and IgG1, and chronic dermatitis with T cell, mast cell, macrophage and eosinophil infiltration. Colonization or infection with S. aureus has been recognized to exacerbate AD and perpetuate chronicity of this skin disease.

AD is often found in patients with asthma and allergic rhinitis, and is frequently the initial manifestation of allergic disease. About 20% of the population in Western countries suffer from these allergic diseases, and the incidence of AD in developed countries is rising for unknown reasons. AD typically begins in childhood and can often persist through adolescence into adulthood. Current treatments for AD include topical corticosteroids, oral cyclosporin A, non-corticosteroid immunosuppressants such as tacrolimus (FK506 in ointment form), and interferon-gamma. Despite the variety of treatments for AD, many patients' symptoms do not improve, or they have adverse reactions to medications, requiring the search for other, more effective therapeutic agents. The FcγRIA polypeptides of the invention can be used in the treatment of specific human diseases such as atopic dermatitis, inflammatory skin conditions, and other conditions disclosed herein.

10. Multiple Sclerosis

Multiple sclerosis is a relatively commonly occurring autoimmune disease characterized by demyelination and chronic inflammation of the central nervous system (CNS). Although the mechanisms underlying disease initiation are not clearly understood, the disease processes that contribute to clinical progression of multiple sclerosis are inflammation, demyelination, and axonal loss, or neurodegeneration. Macrophages and microglia are the main immune cells of the CNS. These cells, as well as T cells, neutrophils, astrocytes, and microglia, can contribute to the immune-related pathology of, e.g., multiple sclerosis. Furthermore, T cell reactivity/autoimmunity to several myelin proteins, including myelin basic protein (MBP), proteolipid protein (PLP), myelin oligodendrocyte protein (MOG), and perhaps other myeline proteins, have been implicated in the induction and perpetuation of disease state and pathology of multiple sclerosis. This interaction of autoreactive T cells and myelin proteins can result in the release of proinflammatory cytokines, including TNF-α, IFN-γ, and IL-17, among others. Additional consequences are the proliferation of T cells, activation of B cells and macrophages, upregulation of chemokines and adhesion molecules, and the disruption of the blood-brain barrier. The ensuing pathology is a loss of oligodendrocytes and axons, and the formation of a demyelinated “plaque”. The plaque consists of a lesion in which the myelin sheath is now absent and the demyelinated axons are embedded within glial scar tissue. Demyelination can also occur as the result of specific recognition and opsinization of myelin antigens by autoantibodies, followed by complement—and/or activated macrophage-mediated destruction. It is this axonal loss and neurodegeneration that is thought to be primarily responsible for the irreversible neurological impairment that is observed in progressive multiple sclerosis.

There is a large amount of clinical and pathological heterogeneity in the course of human multiple sclerosis. Symptoms most often begin between the ages of 18 and 50 years old, but can begin at any age. The clinical symptoms of multiple sclerosis can vary from mild vision disturbances and headaches, to blindness, severe ataxia and paralysis. The majority of the patients (70-75%) have relapsing-remitting multiple sclerosis, in which disease symptoms can recur within a matter of hours to days, followed by a much slower recovery; the absence of symptoms during stages of remission is not uncommon. The incidence and frequency of relapses and remissions can vary greatly, but as time progresses, the recovery phases can be incomplete and slow to occur. This worsening of disease in these cases is classified as secondary-progressive multiple sclerosis, and occurs in approximately 10-15% of multiple sclerosis patients. Another 10-15% of patients are diagnosed with primary-progressive multiple sclerosis, in which disease symptoms and physical impairment progress at a steady rate throughout the disease process.

The FcγRIA polypeptides of the invention could be used in the treatment of multiple sclerosis. If the addition of such polypeptides markedly reduces the production and expression of inflammatory mediators (i.e. CNS-infiltrating immune cells; CNS expression of inflammatory cytokines/chemokines, etc.) and symptoms of multiple sclerosis (e.g. paralysis; ataxia; weight loss, etc.), it would be expected to be efficacious in the treatment of humans.

### IX. Pharmaceutical Compositions

A pharmaceutical composition comprising an FcγRIA polypeptide can be formulated according to known methods to prepare pharmaceutically useful compositions, whereby the therapeutic polypeptide is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, an FcγRIA polypeptide of the invention and a pharmaceutically acceptable carrier are administered to a patient in a therapeutically effective amount. A combination of a therapeutic molecule of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response.

For pharmaceutical use, the FcγRIA polypeptides of the present invention are formulated for parenteral, particularly intravenous or subcutaneous, delivery according to conventional methods. Intravenous administration will be by bolus injection, controlled release, e.g, using mini-pumps or other appropriate technology, or by infusion over a typical period of one to several hours. In general, pharmaceutical formulations will include a hematopoietic protein in combination with a pharmaceutically acceptable vehicle, such as saline, buffered saline, 5% dextrose in water or the like. Formulations may further include one or more excipients, preservatives, solubilizers, buffering agents, albumin to provent protein loss on vial surfaces, etc. When utilizing such a combination therapy, the polypeptides may be combined in a single formulation or may be administered in separate formulations. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 mg/kg of patient weight per day, preferably 0.5-20 mg/kg per day, with the exact dose determined by the clinician according to accepted standards, taking into account the nature and severity of the condition to be treated, patient traits, etc. Determination of dose is within the level of ordinary skill in the art. The proteins will commonly be administered over a period of up to 28 days following chemotherapy or bone-marrow transplant or until a platelet count of >20,000/mm³, preferably >50,000/mm³, is achieved. More commonly, the polypeptides will be administered over one week or less, often over a period of one to three days. In general, a therapeutically effective amount of antibodies of the present invention is an amount sufficient to produce a clinically significant increase in the proliferation and/or differentiation of lymphoid or myeloid progenitor cells, which will be manifested as an increase in circulating levels of mature cells (e.g. platelets or neutrophils). Treatment of platelet disorders will thus be continued until a platelet count of at least 20,000/mm³, preferably 50,000/mm³, is reached. The FcγRIA polypeptides of the present invention can also be administered in combination with other anti-inflammatories. Within regimens of combination therapy, daily doses of other anti-inflammatories are commonly known by one skilled in the art, or can be determined without undue experimentation.

Generally, the dosage of administered FcγRIA polypeptides will vary depending upon such factors as the patient's age, weight, height, sex, general medical condition and previous medical history. Typically, it is desirable to provide the recipient with a dosage of antibodies which is in the range of from about 1 pg/kg to 10 mg/kg (amount of agent/body weight of patient), although a lower or higher dosage also may be administered as circumstances dictate.

Administration of the FcγRIA polypeptides of the invention to a subject can be intravenous, intraarterial, intraperitoneal, intramuscular, subcutaneous, intrapleural, intrathecal, by perfusion through a regional catheter, or by direct intralesional injection. When administering therapeutic polypeptides by injection, the administration may be by continuous infusion or by single or multiple boluses.

Additional routes of administration include oral, mucosal-membrane, pulmonary, and transcutaneous. Oral delivery is suitable for polyester microspheres, zein microspheres, proteinoid microspheres, polycyanoacrylate microspheres, and lipid-based systems (see, for example, DiBase and Morrel, “Oral Delivery of Microencapsulated Proteins,” in Protein Delivery: Physical Systems, Sanders and Hendren (eds.), pages 255-288 (Plenum Press 1997)). The feasibility of an intranasal delivery is exemplified by such a mode of insulin administration (see, e.g., Hinchcliffe and Illum, Adv. Drug Deliv. Rev. 35:199, 1999). Dry or liquid particles comprising antibodies of the invention can be prepared and inhaled with the aid of dry-powder dispersers, liquid aerosol generators, or nebulizers (e.g., Pettit and Gombotz, TIBTECH 16:343, 1998; Patton et al., Adv. Drug Deliv. Rev. 35:235, 1999). This approach is illustrated by the AERX diabetes management system, which is a hand-held electronic inhaler that delivers aerosolized insulin into the lungs. Studies have shown that proteins as large as 48,000 kDa have been delivered across skin at therapeutic concentrations with the aid of low-frequency ultrasound, which illustrates the feasibility of trascutaneous administration (Mitragotri et al., Science 269:850, 1995). Transdermal delivery using electroporation provides another means to administer the FcγRIA polypeptides of the invention (Potts et al., Pharm. Biotechnol. 10:213, 1997).

A pharmaceutical composition comprising an FcγRIA polypeptide of the invention can be formulated according to known methods to prepare pharmaceutically useful compositions, whereby the therapeutic proteins are combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, for example, Gennaro (ed.), Remington's Pharmaceutical Sciences, 19th Edition (Mack Publishing Company 1995).

For purposes of therapy, the FcγRIA polypeptides of the invention and a pharmaceutically acceptable carrier are administered to a patient in a therapeutically effective amount. A combination of an FcγRIA polypeptide of the present invention and a pharmaceutically acceptable carrier is said to be administered in a “therapeutically effective amount” if the amount administered is physiologically significant. An agent is physiologically significant if its presence results in a detectable change in the physiology of a recipient patient. For example, an agent used to treat inflammation is physiologically significant if its presence alleviates the inflammatory response. Effective treatment may be assessed in a variety of ways. In one embodiment, effective treatment is determined by reduced inflammation. In other embodiments, effective treatment is marked by inhibition of inflammation. In still other embodiments, effective therapy is measured by increased well-being of the patient including such signs as weight gain, regained strength, decreased pain, thriving, and subjective indications from the patient of better health.

A pharmaceutical composition comprising antibodies of the invention can be furnished in liquid form, in an aerosol, or in solid form. Liquid forms, are illustrated by injectable solutions and oral suspensions. Exemplary solid forms include capsules, tablets, and controlled-release forms. The latter form is illustrated by miniosmotic pumps and implants (Bremer et al., Pharm. Biotechnol. 10:239, 1997; Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)).

Liposomes provide one means to deliver the FcγRIA polypeptides of the invention to a subject intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments (see generally Bakker-Woudenberg et al., Eur. J. Clin. Microbiol. Infect. Dis. 12 (Suppl. 1):S61, 1993; Kim, Drugs 46:618, 1993; Ranade, “Site-Specific Drug Delivery Using Liposomes as Carriers,” in Drug Delivery Systems, Ranade and Hollinger (eds.), pages 3-24 (CRC Press 1995)). Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s) (see, e.g., Machy et al., Liposomes In Cell Biology And Pharmacology (John Libbey 1987), and Ostro et al., American J. Hosp. Pharm. 46:1576, 1989). Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446:368, 1985). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (Claassen et al., Biochim. Biophys. Acta 802:428, 1984). In addition, incorporation of glycolipid- or polyethelene glycol-derivatized phospholipids into liposome membranes has been shown to result in a significantly reduced uptake by the reticuloendothelial system (Allen et al., Biochim. Biophys. Acta 1068:133, 1991; Allen et al., Biochim. Biophys. Acta 1150:9, 1993).

Liposomes can also be prepared to target particular cells or organs by varying phospholipid composition or by inserting receptors or ligands into the liposomes. For example, liposomes, prepared with a high content of a nonionic surfactant, have been used to target the liver (Hayakawa et al., Japanese Patent 04-244,018; Kato et al., Biol. Pharm. Bull. 16:960, 1993). These formulations were prepared by mixing soybean phospatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver (Shimizu et al., Biol. Pharm. Bull. 20:881, 1997).

Alternatively, various targeting ligands can be bound to the surface of the liposome, such as antibodies, antibody fragments, carbohydrates, vitamins, and transport proteins. For example, liposomes can be modified with branched type galactosyllipid derivatives to target asialoglycoprotein (galactose) receptors, which are exclusively expressed on the surface of liver cells (Kato and Sugiyama, Crit. Rev. Ther. Drug Carrier Syst. 14:287, 1997; Murahashi et al., Biol. Pharm. Bull. 20:259, 1997). Similarly, Wu et al. (Hepatology 27:772, 1998) have shown that labeling liposomes with asialofetuin led to a shortened liposome plasma half-life and greatly enhanced uptake of asialofetuin-labeled liposome by hepatocytes. On the other hand, hepatic accumulation of liposomes comprising branched type galactosyllipid derivatives can be inhibited by preinjection of asialofetuin (Murahashi et al., Biol. Pharm. Bull. 20:259, 1997). Polyaconitylated human serum albumin liposomes provide another approach for targeting liposomes to liver cells (Kamps et al., Proc. Nat'l Acad. Sci. USA 94:11681, 1997). Moreover, Geho, et al. U.S. Pat. No. 4,603,044, describe a hepatocyte-directed liposome vesicle delivery system, which has specificity for hepatobiliary receptors associated with the specialized metabolic cells of the liver.

In a more general approach to tissue targeting, target cells are prelabeled with biotinylated antibodies specific for a ligand expressed by the target cell (Harasym et al., Adv. Drug Deliv. Rev. 32:99, 1998). After plasma elimination of free antibody, streptavidin-conjugated liposomes are administered. In another approach, targeting antibodies are directly attached to liposomes (Harasym et al., Adv. Drug Deliv. Rev. 32:99, 1998).

The FcγRIA polypeptides of the invention can be encapsulated within liposomes using standard techniques of protein microencapsulation (see, e.g., Anderson et al., Infect. Immun. 31:1099, 1981; Anderson et al., Cancer Res. 50:1853, 1990; Cohen et al., Biochim. Biophys. Acta 1063:95, 1991; Alving et al. “Preparation and Use of Liposomes in Immunological Studies,” in Liposome Technology, 2nd Edition, Vol. III, Gregoriadis (ed.), page 317 (CRC Press 1993); Wassef et al., Meth. Enzymol. 149:124, 1987). As noted above, therapeutically useful liposomes may contain a variety of components. For example, liposomes may comprise lipid derivatives of poly(ethylene glycol) (Allen et al., Biochim. Biophys. Acta 1150:9, 1993).

Degradable polymer microspheres have been designed to maintain high systemic levels of therapeutic proteins. Microspheres are prepared from degradable polymers such as poly(lactide-co-glycolide) (PLG), polyanhydrides, poly(ortho esters), nonbiodegradable ethylvinyl acetate polymers, in which proteins are entrapped in the polymer (Gombotz and Pettit, Bioconjugate Chem. 6:332, 1995; Ranade, “Role of Polymers in Drug Delivery,” in Drug Delivery Systems, Ranade and Hollinger (eds.), pages 51-93 (CRC Press 1995); Roskos and Maskiewicz, “Degradable Controlled Release Systems Useful for Protein Delivery,” in Protein Delivery: Physical Systems, Sanders and Hendren (eds.), pages 45-92 (Plenum Press 1997); Bartus et al., Science 281:1161, 1998; Putney and Burke, Nature Biotechnology 16:153, 1998; Putney, Curr. Opin. Chem. Biol. 2:548, 1998). Polyethylene glycol (PEG)-coated nanospheres can also provide carriers for intravenous administration of therapeutic proteins (see, e.g., Gref et al., Pharm. Biotechnol. 10: 167, 1997).

The present invention also contemplates chemically modified FcγRIA polypeptides in which a polypeptide is linked with a polymer, as discussed above.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

As an illustration, pharmaceutical compositions may be supplied as a kit comprising a container that comprises an FcγRIA polypeptide of the invention. The FcγRIA polypeptides of the invention can be provided in the form of an injectable solution for single or multiple doses, or as a sterile powder that will be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical composition. Moreover, such information may include a statement that the FcγRIA polypeptide-comprising composition is contraindicated in patients with known hypersensitivity to FcγRIA.

A pharmaceutical composition comprising the FcγRIA polypeptides of the invention can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants (Bremer et al., Pharm. Biotechnol. 10:239, 1997; Ranade, “Implants in Drug Delivery,” in Drug Delivery Systems, Ranade and Hollinger (eds.), pages 95-123 (CRC Press 1995); Bremer et al., “Protein Delivery with Infusion Pumps,” in Protein Delivery: Physical Systems, Sanders and Hendren (eds.), pages 239-254 (Plenum Press 1997); Yewey et al., “Delivery of Proteins from a Controlled Release Injectable Implant,” in Protein Delivery: Physical Systems, Sanders and Hendren (eds.), pages 93-117 (Plenum Press 1997)). Other solid forms include creams, pastes, other topological applications, and the like.

Other dosage forms can be devised by those skilled in the art, as shown, for example, by Ansel and Popovich, Pharmaceutical Dosage Forms and Drug Delivery Systems, 5^{th} Edition (Lea & Febiger 1990), Gennaro (ed.), Remington's Pharmaceutical Sciences, 19^{th} Edition (Mack Publishing Company 1995), and by Ranade and Hollinger, Drug Delivery Systems (CRC Press 1996).

The present invention contemplates FcγRIA polypeptides and methods and therapeutic uses comprising such polypeptides as described herein. Such compositions can further comprise a carrier. The carrier can be a conventional organic or inorganic carrier. Examples of carriers include water, buffer solution, alcohol, propylene glycol, macrogol, sesame oil, corn oil, and the like.

From the foregoing, it will be appreciated that, although specific embodiments of the invention have been described herein for purposes of illustration, various modifications may be made without deviating from the spirit and scope of the invention. Accordingly, the invention is further illustrated by the following non-limiting examples and is not limited except as by the appended claims.

### EXAMPLE 1

### Construction of Mammalian FcγRIA-Fc5 Expression Construct

### A. Mammalian FcγRIA-Fc5 Expression Construct

An expression plasmid encoding FcγRIA-Fc5 is constructed via homologous recombination in yeast with two DNA fragments encoding an extracellular fragment of FcγRIA (amino acids 1 to 282 of SEQ ID NO:2) and the (Gly₄Ser)₃ linker-Fc5 inserted into mammalian expression vector, pZMP40. Fc5 is an effector minus form of human gamma1 Fc and the linker is present as a spacer between the fusion partners. pZMP40 is a derivative of plasmid pZMP21, made by modifying the multiple cloning site. pZMP21 is described in US patent application US 2003/0232414 A1, deposited at the American Type Culture Collection, 10801 University Boulevard, Manassas, Va. 20110-2209, designated No. PTA-5266.

The indicated fragment of FcγRIA cDNA (residues 1-1122 of the native cDNA sequence) is isolated using PCR. The upstream primer for PCR includes from 5′ to 3′ end: 40 bp of flanking sequence from the vector and 21 bp corresponding to the amino terminus from the open reading frame of FcγRIA (zc54568 5′-TCTCCACAGGTGTCCAGGGAATTCATATAGGCCGGCC ACCATGTGGTTCTTGACAACTCTG-3′; SEQ ID NO:6). The downstream primer for the FcγRIA half of the fusion protein consists from 5′ to 3′ of the bottom strand sequence of 40 bp of (Gly₄Ser)₃ linker of an irrelevant (Gly₄Ser)₃ Fc5 fusion protein sequence and the last 21 bp of the FcγRIA extracellular domain, 1102 to 1122 (zc54539 5′-CGCCTCCACCGCTTCCACCCCCGCCGGAG CCCCCACCTCCCGTGGCCCCCTGGGGCTCCTT-3′; SEQ ID NO:7). The Fc5 moiety with the (Gly₄Ser)₃ linker, was made with an upstream primer including from 5′ to 3′: 40 bp of flanking sequence from the FcγRIa extracellular domain and 21 bp corresponding to the amino terminus of the (Gly₄Ser)₃ Fc5 partner (zc54537 5′-GCCTGTGACCATCACTGTCCAAGTGCCCAGCATGGGCAG CGGAGGTGGGGGCTCCGGCGGG-3′; SEQ ID NO:8). The downstream primer for the Fc5 portion of the fusion protein consists from 5′ to 3′ of the bottom strand sequence of 40 bp of the flanking sequence from the vector, pZMP40 and the last 21 bp of Fc5 (zc54578 5′-CAACCCCAGA GCTGTTTTAAGGCGCGCCTCTAGATTATTTTTATTTACCCGGAGACAGGGAGAG-3′; SEQ ID NO:9).

The PCR amplification reaction condition is as follows: 1 cycle, 94° C., 5 minutes; 25 cycles, 94° C., 1 minute, followed by 65° C., 1 minute, followed by 72° C., 1 minute; 1 cycle, 72° C., 5 minutes. Ten μl of each 100 μl PCR reaction is run on a 0.8% LMP agarose gel (Seaplaque GTG) with 1×TBE buffer for analysis. The plasmid pZMP40, which has been cut with BglII, is used for homologous recombination with the PCR fragments. The remaining 90 μl of each PCR reaction and 200 ng of cut pZMP40 are precipitated with the addition of 20 μl M Na Acetate and 500 μl of absolute ethanol, rinsed, dried and resuspended in 10 μl water.

One hundred μl of competent yeast cells (S. cerevisiae) are combined with 10 μl of the DNA mixture from above and transferred to a 0.2 cm electroporation cuvette. The yeast/DNA mixtures are electropulsed at 0.75 kV (5 kV/cm), ∞ ohms, 25 μF. To each cuvette is added 600 μl of 1.2 M sorbitol and the yeast is plated in two 300 μl aliquots onto two URA-D plates and incubated at 30° C. After about 48 hours, approximately 50 μl packed yeast cells taken from the Ura+ yeast transformants of a single plate are resuspended in 100 μl of lysis buffer (2% Triton X-100, 1% SDS, 100 mM NaCl, 10 mM Tris, pH 8.0, 1 mM EDTA), 100 μl of Qiagen P1 buffer from a Qiagen miniprep kit (Invitrogen, Carlsbad, Calif.) and 20 U of Zymolyase (Zymo Research, Orange, Calif., catalog #1001). This mixture is incubated for 30 minutes at 37° C., then the remainder of the Qiagen miniprep protocol is performed. The plasmid DNA is eluted twice in 100 μl water and precipitated with 20 μl 3 M Na Acetate and 500 μl absolute ethanol. The pellet is rinsed once with 70% ethanol, air-dried and resuspended in 10 μl water for transformation.

Fifty μl electrocompetent E. coli cells (DH10B, Invitrogen, Carlsbad, Calif.) are transformed with 2 μl yeast DNA. The cells are electropulsed at 1.7 kV, 25 μF and 400 ohms. Following electroporation, 1 ml SOC (2% Bacto Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is plated in 250, 100 and 10 μl aliquots on three LB AMP plates (LB broth (Lennox), 1.8% Bacto Agar (Difco), 100 mg/L Ampicillin).

Individual clones harboring the correct expression construct for FcγRIA-Fc5 are identified by restriction digest to verify the presence of the insert and to confirm that the various DNA sequences have been joined correctly to one another. The inserts of positive clones are subjected to sequence analysis. Larger scale plasmid DNA is isolated using the Qiagen Maxi kit (Qiagen) according to manufacturer's instruction.

The FcγRIA-Fc5 nucleotide coding sequence is set forth in SEQ ID NO:10. The corresponding amino acid sequence of the encoded FcγRIA-Fc5 polypeptide is shown in SEQ ID NO:1.

### B. Transfection and Selection of FcγRIA-Fc5 Constructs in CHO Cells

Three sets of 200 μg of the FcγRIA-Fc5 constructs are separately digested with 200 units of PvuI at 37° C. for three hours, precipitated with ethanol, and centrifuged in a 1.5 mL microfuge tube. The supernatant is decanted off the pellet, and the pellet is washed with 300 μl of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 μF, high capacitance, at 300 V. The contents of the cuvettes are then removed, pooled, and diluted to 25 mL with CHO cell tissue culture medium and placed in a 125 mL shake flask. The flask is placed in an incubator on a shaker at 37° C., 6% CO₂ with shaking at 120 rpm.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### EXAMPLE 2

### Construction of Mammalian Soluble FcγRIA Expression Constructs that Express FcγRIA-CEE, FcγRIA-CHIS, and FcγRIA-CFLAG Tagged Proteins

An expression construct containing the extracellular domain of human FcγRIA with a C-terminal tag, either Glu-Glu (CEE), six His (CHIS), or FLAG (CFLAG), is constructed via PCR and homologous recombination using a DNA fragment encoding FcγRIA (SEQ ID NO:12) and the expression vector pZMP20.

The PCR fragment encoding FcγRIA-CEE contains a 5′ overlap with the pZMP20 vector sequence in the 5′ non-translated region, an FcγRIA extracellular domain coding region portion of SEQ ID NO:12 (nucleotides 1-846), the Glu-Glu tag (Glu Glu Tyr Met Pro Met Glu; SEQ ID NO:13) coding sequence, and a 3′ overlap with the pZMP20 vector in the poliovirus internal ribosome entry site region. The PCR amplification reaction uses the 5′ oligonucleotide “100” (ACAGGTGTCCAGGGAATTCATATAGGCCGGCCACCATGTGGTTCTTGACAACTCTG; SEQ ID NO:14), the 3′ oligonucleotide “200” (CAACCCCAGAGCTGTTTTAAGGCGCGCCTCTAGAT TATTCCATGGGCATGTATTCTTCCACTTGAAGCTCCAACTCAGG; SEQ ID NO:15), and a previously generated DNA clone of FcγRIA as the template (SEQ ID NO:12).

The PCR amplification reaction condition is as follows: 1 cycle, 94° C., 5 minutes; 35 cycles, 94° C., 1 minute, followed by 55° C., 2 minutes, followed by 72° C., 3 minutes; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704).

Plasmid pZMP20 is a mammalian expression vector containing an expression cassette having the chimeric CMV enhancer/MPSV promoter, a BglII site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP20 is digested with BglII prior to recombination in yeast with the gel extracted FcγRIA-CEE PCR fragment. One hundred μl of competent yeast (S. cerevisiae) cells are combined with 10 μl of the FcγRIA-CEE insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), cc ohms, and 25 μF. Six hundred μl of 1.2 M sorbitol is added to the cuvette, and the yeast is plated in 100 μl and 300 μl aliquots onto two URA-D plates and incubated at 30° C. After about 72 hours, the Ura+ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred μl of the lysis mixture is added to an Eppendorf tube containing 250 μl acid-washed glass beads and 300 μl phenol-chloroform, is vortexed for 3 minutes, and spun for 5 minutes in an Eppendorf centrifuge at maximum speed. Three hundred μl of the aqueous phase is transferred to a fresh tube, and the DNA is precipitated with 600 μl ethanol, followed by centrifugation for 30 minutes at maximum speed. The tube is decanted and the pellet is washed with 1 mL of 70% ethanol. The tube is decanted and the DNA pellet is resuspended in 30 μl 10 mM Tris, pH 8.0, 1 mM EDTA.

Transformation of electrocompetent E. coli host cells (DH12S) is done using 5 μl of the yeast DNA preparation and 50 μl of E. coli cells. The cells are electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 μl and 200 μl aliquots on two LB AMP plates (LB broth (Lennox), 1.8% Bacto™ Agar (Difco), 100 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions.

The same process is used to prepare the FcγRIA with a C-terminal his tag, composed of Gly Ser Gly Gly His His His His His His (SEQ ID NO:16) (FcγRIA-CHIS) or the C-terminal FLAG tag, composed of Gly Ser Asp Tyr Lys Asp Asp Asp Asp Lys (SEQ ID NO:17) (FcγRIA-CFLAG). To prepare these constructs, instead of the 3′ oligonucleotide “200”, the 3′ oligonucleotide “300” (CAACCCCAGAGCTGTTTTAAGGCGCGCCTCTAGATTAGTGATGGTGATGGTGATG TCCACCAGATCCCACTTGAAGCTCCAACTCAGG; SEQ ID NO:18) is used to generate FcγRIA-CHIS or the 3′ oligonucleotide “400” (CAACCCCAGAGCTGTTTTAAGGCGCGCCTCTAGATTA CTTATCATCATCATCCTTATAATCGGATCCCACTTGAAGCTCCAACTCAGG; SEQ ID NO:19) is used to generate FcγRIA-CFLAG.

### EXAMPLE 3

### Transfection and Expression of Soluble FcγRIA Receptor Expression Constructs that Express the FcγRIA-CEE, FcγRIA-CHIS, and FcγRIA-CFLAG C-Terminal Tagged Proteins

Three sets of 200 μg of each of the soluble FcγRIA tagged expression constructs are separately digested with 200 units of PvuI at 37° C. for three hours, precipitated with isopropyl alcohol, and centrifuged in a 1.5 mL microfuge tube. The supernatant is decanted off the pellet, and the pellet is washed with 1 mL of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 μF, high capacitance, at 300 V. The contents of the cuvettes are then removed, pooled, and diluted to 25 mL with CHO cell tissue culture medium and placed in a 125 mL shake flask. The flask is placed in an incubator on a shaker at 37° C., 6% CO₂ with shaking at 120 RPM.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### EXAMPLE 4

### Construction of Mammalian Soluble FcγRIb1 Expression Constructs that Express FcγRIb1-CEE, FcγRIb1-CHIS, and FcγRIb1-CFLAG Tagged Proteins

An expression construct containing the extracellular domain of human FcγRIb1 with a C-terminal tag, either Glu-Glu (CEE), six His (CHIS), or FLAG (CFLAG), is constructed via PCR and homologous recombination using a DNA fragment encoding FcγRIb1 (SEQ ID NO: 20) and the expression vector pZMP20.

The PCR fragment encoding FcγRIb1-CEE contains a 5′ overlap with the pZMP20 vector sequence in the 5′ non-translated region, the FcγRIb1 extracellular domain coding region portion of SEQ ID NO: 20 (nucleotides 1-570), the Glu-Glu tag (Glu Glu Tyr Met Pro Met Glu; SEQ ID NO:13) coding sequence, and a 3′ overlap with the pZMP20 vector in the poliovirus internal ribosome entry site region. The PCR amplification reaction uses the 5′ oligonucleotide “100” (ACAGGTGTCCAGGGAATTCATATAGGCCGGCCACCATGTGGTTCTTGACAACTCTG; SEQ ID NO:21), the 3′ oligonucleotide “211” (CAACCCCAGAGCTGTTTTAAGGCGCGCCTCTAGAT TATTCCATGGGCATGTATTCTTCAAATAGCTCTTTCACAGTGTA; SEQ ID NO:22), and a previously generated DNA clone of FcγRIb1 as the template (SEQ ID NO:20).

The PCR amplification reaction condition is as follows: 1 cycle, 94° C., 5 minutes; 35 cycles, 94° C., 1 minute, followed by 55° C., 2 minutes, followed by 72° C., 3 minutes; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704).

Plasmid pZMP20 is a mammalian expression vector containing an expression cassette having the chimeric CMV enhancer/MPSV promoter, a BglII site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP20 is digested with BglII prior to recombination in yeast with the gel extracted FcγRIb1-CEE PCR fragment. One hundred μl of competent yeast (S. cerevisiae) cells are combined with 10 μl of the FcγRIb1-CEE insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), cc ohms, and 25 μF. Six hundred μl of 1.2 M sorbitol is added to the cuvette, and the yeast is plated in 100 μl and 300 μl aliquots onto two URA-D plates and incubated at 30° C. After about 72 hours, the Ura+ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred μl of the lysis mixture is added to an Eppendorf tube containing 250 μl acid-washed glass beads and 300 μl phenol-chloroform, is vortexed for 3 minutes, and spun for 5 minutes in an Eppendorf centrifuge at maximum speed. Three hundred μl of the aqueous phase is transferred to a fresh tube, and the DNA is precipitated with 600 μl ethanol, followed by centrifugation for 30 minutes at maximum speed. The tube is decanted and the pellet is washed with 1 mL of 70% ethanol. The tube is decanted and the DNA pellet is resuspended in 30 μl 10 mM Tris, pH 8.0, 1 mM EDTA.

Transformation of electrocompetent E. coli host cells (DH12S) is done using 5 μl of the yeast DNA preparation and 50 μl of E. coli cells. The cells are electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 μl and 200 μl aliquots on two LB AMP plates (LB broth (Lennox), 1.8% Bacto™ Agar (Difco), 100 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions.

The same process is used to prepare the FcγRIb1 with a C-terminal his tag, composed of Gly Ser Gly Gly His His His His His His (FcγRIb1-CHIS) or the C-terminal FLAG tag, composed of Gly Ser Asp Tyr Lys Asp Asp Asp Asp Lys (FcγRIb1-CFLAG). To prepare these constructs, instead of the 3′ oligonucleotide “211”, the 3′ oligonucleotide “311” (CAACCCCAGAGCTGTTTT AAGGCGCGCCTCTAGATTAGTGATGGTGATGGTGATGTCCACCAGATCCAAATAGCTCTT TCACAGTGTA; SEQ ID NO:23) is used to generate FcγRIb1-CHIS or the 3′ oligonucleotide “411” (CAACCCCAGAGCTGTTTTAAGGCGCGCCTCTAGATTACTTATCATCATCATCCTTATAAT CGGATCCAAATAGCTCTTTCACAGTGTA; SEQ ID NO:24) is used to generate FcγRIb1CFLAG.

### EXAMPLE 5

### Transfection and Expression of Soluble FcγRIb1 Receptor Expression Constructs that Express the FcγRIb1-CEE, FcγRIb1-CHIS, and FcγRIb1-CFLAG C-Terminal Tagged Proteins

Three sets of 200 μg of each of the soluble FcγRIb1 tagged expression constructs are separately digested with 200 units of PvuI at 37° C. for three hours, precipitated with isopropyl alcohol, and centrifuged in a 1.5 mL microfuge tube. The supernatant is decanted off the pellet, and the pellet is washed with 1 mL of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 μF, high capacitance, at 300 V. The contents of the cuvettes are then removed, pooled, and diluted to 25 mL with CHO cell tissue culture medium and placed in a 125 mL shake flask. The flask is placed in an incubator on a shaker at 37° C., 6% CO₂ with shaking at 120 RPM.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### EXAMPLE 6

### Construction of Mammalian Soluble FcγRIc Expression Constructs that Express FcγRIc-CEE, FcγRIc-CHIS, and FcγRIc-CFLAG Tagged Proteins

An expression construct containing the extracellular domain of human FcγRIc with a C-terminal tag, either Glu-Glu (CEE), six His (CHIS), or FLAG (CFLAG), is constructed via PCR and homologous recombination using a DNA fragment encoding FcγRIc (SEQ ID NO:25) and the expression vector pZMP20.

The PCR fragment encoding FcγRIc-CEE contains a 5′ overlap with the pZMP20 vector sequence in the 5′ non-translated region, the FcγRIc extracellular domain coding region portion of SEQ ID NO:25 (nucleotides 1-570), the Glu-Glu tag (Glu Glu Tyr Met Pro Met Glu; SEQ ID NO:13) coding sequence, and a 3′ overlap with the pZMP20 vector in the poliovirus internal ribosome entry site region. The PCR amplification reaction uses the 5′ oligonucleotide “100” (ACAGGTGTCCAGGGAATTCATATAGGCCGGCCACCATGTGGTTCTTGACAACTCTG; SEQ ID NO:26), the 3′ oligonucleotide “211” (CAACCCCAGAGCTGTTTTAAGGCGCGCCTCTAGAT TATTCCATGGGCATGTATTCTTCAAATAGCTCTTTCACAGTGTA; SEQ ID NO:27), and a previously generated DNA clone of FcγRIc as the template (SEQ ID NO:25).

The PCR amplification reaction condition is as follows: 1 cycle, 94° C., 5 minutes; 35 cycles, 94° C., 1 minute, followed by 55° C., 2 minutes, followed by 72° C., 3 minutes; 1 cycle, 72° C., 10 minutes. The PCR reaction mixture is run on a 1% agarose gel and the DNA fragment corresponding to the expected size is extracted from the gel using a QIAquick™ Gel Extraction Kit (Qiagen, Cat. No. 28704).

Plasmid pZMP20 is a mammalian expression vector containing an expression cassette having the chimeric CMV enhancer/MPSV promoter, a BglII site for linearization prior to yeast recombination, an internal ribosome entry element from poliovirus, the extracellular domain of CD8 truncated at the C-terminal end of the transmembrane domain; an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP20 is digested with BglII prior to recombination in yeast with the gel extracted FcγRIc-CEE PCR fragment. One hundred μl of competent yeast (S. cerevisiae) cells are combined with 10 μl of the FcγRIcCEE insert DNA and 100 ng of BglII digested pZMP20 vector, and the mix is transferred to a 0.2 cm electroporation cuvette. The yeast/DNA mixture is electropulsed using power supply (BioRad Laboratories, Hercules, Calif.) settings of 0.75 kV (5 kV/cm), cc ohms, and 25 μF. Six hundred μl of 1.2 M sorbitol is added to the cuvette, and the yeast is plated in 100 μl and 300 μl aliquots onto two URA-D plates and incubated at 30° C. After about 72 hours, the Ura+ yeast transformants from a single plate are resuspended in 1 ml H₂O and spun briefly to pellet the yeast cells. The cell pellet is resuspended in 0.5 ml of lysis buffer (2% Triton X-100, 1% SDS, 100 mM NaCl, 10 mM Tris, pH 8.0, 1 mM EDTA). The five hundred μl of the lysis mixture is added to an Eppendorf tube containing 250 μl acid-washed glass beads and 300 μl phenol-chloroform, is vortexed for 3 minutes, and spun for 5 minutes in an Eppendorf centrifuge at maximum speed. Three hundred μl of the aqueous phase is transferred to a fresh tube, and the DNA is precipitated with 600 μl ethanol, followed by centrifugation for 30 minutes at maximum speed. The tube is decanted and the pellet is washed with 1 mL of 70% ethanol. The tube is decanted and the DNA pellet is resuspended in 30 μl 10 mM Tris, pH 8.0, 1 mM EDTA.

Transformation of electrocompetent E. coli host cells (DH12S) is done using 5 μl of the yeast DNA preparation and 50 μl of E. coli cells. The cells are electropulsed at 2.0 kV, 25 μF, and 400 ohms. Following electroporation, 1 ml SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) is added and then the cells are plated in 50 μl and 200 μl aliquots on two LB AMP plates (LB broth (Lennox), 1.8% Bacto™ Agar (Difco), 100 mg/L Ampicillin).

The inserts of three DNA clones for the construct are subjected to sequence analysis and one clone containing the correct sequence is selected. Large-scale plasmid DNA is isolated using a commercially available kit (QIAGEN Plasmid Mega Kit, Qiagen, Valencia, Calif.) according to manufacturer's instructions.

The same process is used to prepare the FcγRIc with a C-terminal his tag, composed of Gly Ser Gly Gly His His His His His His (FcγRIcCHIS) or the C-terminal FLAG tag, composed of Gly Ser Asp Tyr Lys Asp Asp Asp Asp Lys (FcγRIcCFLAG). To prepare these constructs, instead of the 3′ oligonucleotide “211”, the 3′ oligonucleotide “311” (CAACCCCAGAGCTGTTTTAAGGCG CGCCTCTAGATTAGTGATGGTGATGGTGATGTCCACCAGATCCAAATAGCTCTTTCACAG TGTA; SEQ ID NO:28) is used to generate FcγRIcCHIS or the 3′ oligonucleotide “411” (CAACCCCAGAGCTGTTTTAAGGCGCGCCTCTAGATTACTTATCATCATCATCCTTATAAT CGGATCCAAATAGCTCTTTCACAGTGTA; SEQ ID NO:29) is used to generate FcγRIc-CFLAG.

### EXAMPLE 7

### Transfection and Expression of Soluble FcγRIc Receptor Expression Constructs that Express the FcγRIA-CEE, FcγRIc-CHIS, and FcγRIc-CFLAG C-Terminal Tagged Proteins

Three sets of 200 μg of each of the soluble FcγRIc tagged expression constructs are separately digested with 200 units of PvuI at 37° C. for three hours, precipitated with isopropyl alcohol, and centrifuged in a 1.5 mL microfuge tube. The supernatant is decanted off the pellet, and the pellet is washed with 1 mL of 70% ethanol and allowed to incubate for 5 minutes at room temperature. The tube is spun in a microfuge for 10 minutes at 14,000 RPM and the supernatant is decanted off the pellet. The pellet is then resuspended in 750 μl of CHO cell tissue culture medium in a sterile environment, allowed to incubate at 60° C. for 30 minutes, and is allowed to cool to room temperature. Approximately 5×10⁶ CHO cells are pelleted in each of three tubes and are resuspended using the DNA-medium solution. The DNA/cell mixtures are placed in a 0.4 cm gap cuvette and electroporated using the following parameters; 950 μF, high capacitance, at 300 V. The contents of the cuvettes are then removed, pooled, and diluted to 25 mL with CHO cell tissue culture medium and placed in a 125 mL shake flask. The flask is placed in an incubator on a shaker at 37° C., 6% CO₂ with shaking at 120 RPM.

The CHO cells are subjected to nutrient selection followed by step amplification to 200 nM methotrexate (MTX), and then to 1 μM MTX. Tagged protein expression is confirmed by Western blot, and the CHO cell pool is scaled-up for harvests for protein purification.

### EXAMPLE 8

### Purification of FcγRIA-CH6

An expression construct containing the extracellular domain of human FcγRIA with a C-terminal six His (CHIS) tag was constructed as described in Example 2, supra. This construct was transfected into and expressed in CHO cells as described in Example 3, supra. The encoded His-tagged FcγRIA, referred to in the Examples above as “FcγRIA-CHIS,” is also referred to herein as “FcγRIA-CH6” or “pFCGR1A CH6.” The nucleotide coding sequence for FcγRIA-CH6 is shown in SEQ ID NO:30, and the corresponding FcγRIA-CH6 amino acid sequence is shown in SEQ ID NO:31. The expressed FcγRIA-CH6 was purified as described below.

FcγRIA-CH6 was purified from CHO conditioned media by a combination of Ni IMAC capture, chromatography on Q Sepharose, and size exclusion chromatography on Superdex 200. Ni IMAC capture: CHO conditioned media was sterile filtered (0.22 μm) and concentrated 10× using a peristaltic pump system equipped with 10 kD MWCO 0.1 m² membrane. Concentrated media was buffer exchanged with at least 5 CV of 50 mM NaPO₄, 500 mM NaCl pH 7.5 and was adjusted to a final concentration of 25 mM imidazole. The pH was adjusted to 7.5 using either concentrated NaOH or HCl, if necessary. The His-tagged FcγRIA protein was captured using IMAC binding to Ni-NTA His Bind Superflow resin. Prior to application of media, the resin was equilibrated in 50 mM NaPO₄, 500 mM NaCl, 25 mM Imidazole pH 7.5. Binding was allowed to occur overnight at 4° C. in either batch mode using an appropriately sized roller bottle or column mode using a chromatography station. Following the load, the resin was washed with at least 10 CV of 50 mM NaPO₄, 500 mM NaCl, and 25 mM Imidazole pH 7.5. Elution of bound protein was accomplished using either a gradient or steps of increasing imidazole concentration in 50 mM NaPO₄, 500 mM NaCl pH 7.5, with 500 mM Imidazole being the end point in the elution. Fractions were collected and analyzed by western blotting, SDS-PAGE, and RP-HPLC and fractions containing FcγRIA-CH6 were combined.

Q Sepharose Passive Chromatography: The IMAC pool containing FcγRIA-CH6 was buffer exchanged with 15CV into 50 mM NaPO₄, 150 mM NaCl pH 7.5 through the use of a Labscale TFF system equipped with 3×10 kD MWCO 0.1 cm² membranes. A 1.0 mL sample of Q Sepharose resin per 7.5 mg of FcγRIA-CH6 was charged using at least 10CV of 50 mM NaPO₄, 2M NaCl pH 7.5, and then equilibrated with 10 CV of 50 mM NaPO₄, 150 mM NaCl pH 7.5. Resin and the adjusted IMAC pool were combined and incubated overnight at 4° C. with gentle agitation. The slurry was transferred to a gravity flow column, the flow-through was collected and the column was washed with at least 5CV of equilibration buffer. The flow-through and wash fractions were combined and assessed for the presence of FcGγRIA-CH6 by RP-HPLC and SDS-PAGE.

Size Exclusion Chromatography: The Q Sepharose flow-through+wash fraction was concentrated at least 10- to 20-fold using either the TFF labscale system equipped with a 10 kD MWCO 0.1 cm² membrane, a stirred cell system equipped with a YM30 membrane of appropriate diameter, or a 30 kD MWCO Ultracel centrifugal membrane, depending on the fraction volume. The concentrated FcγRIA-CH6 fraction was injected over a Superdex 200 column of appropriate size for the amount of volume and mass injected. The column was equilibrated in formulation buffer which contained 50 mM NaPO₄, 109 mM NaCl, pH 7.3 or 35 mM NaPO₄, 120 mM NaCl pH 7.2. The column was eluted isocratically at a flow rate no greater than 45 cm/hr, fractions were collected and analyzed for the presence of FcγRIA-CH6 by SDS-PAGE and RP-HPLC. Fractions containing FcγRIA-CH6 were combined and concentrated to the desired concentration using a stirred cell apparatus equipped with a YM30 membrane (30 kD MWCO). The final FcγRIA-CH6 concentrate was filtered through a 0.22 um sterile filter and stored at −80° C. until use.

### EXAMPLE 9

### Construction, Expression, and Purification of Soluble FcγRIIA-CH6 and FcγRIIIA-CH6

In addition to construction, expression, and purification of a soluble monomeric form of FcγRIA with a C-terminal His6 tag as described above in Examples 2, 3, and 8, supra, soluble monomeric forms of FcγRIIA and FcγRIIIA were also generated using similar methods.

Briefly, expression constructs encoding soluble monomeric forms of the FcγRIIA and FcγRIIIA were generated using DNA sequences encoding their native signal sequence, their extracellular domain, and a C-terminal His6 tag (GSGGHHHHHH; SEQ ID NO:16). The DNA sequence encoded amino acids 1-212 for FcγRIIA (amino acids 1-212 of SEQ ID NO:33) and 1-195 for FcγRIIIA (amino acids 1-195 of SEQ ID NO:35). Receptors were purified from supernatants derived from Chinese hamster ovary (CHO) DXB-11 cells (Larry Chasin, Columbia University, New York, N.Y.). CHO-conditioned media were sterile filtered, concentrated, and buffer exchanged into 50 mM NaPO₄, 500 mM NaCl, 25 mM imidazole, pH 7.5 (Buffer A). The His-tagged FcγR proteins (FcγRIIA-CH6 and FcγRIIIA-CH6) were captured using Ni-NTA His Bind Superflow resin (Novagen, Madison, Wis.) equilibrated in Buffer A. Elution of bound protein was accomplished using a gradient of imidazole (0-500 mM) in 50 mM NaPO₄, 500 mM NaCl, pH 7.5. Fractions were analyzed for soluble FcγR by SDS-PAGE and Western blotting (anti-6× Histidine HRP mouse IgG1, R & D Systems, Minneapolis, Minn.).

The Ni-NTA fractions containing soluble FcγR were buffer-exchanged into 50 mM NaPO₄, 150 mM NaCl, pH 7.5 (Buffer B) and incubated with Q Sepharose 4FF resin (GE Healthcare, Uppsala, Sweden) that was pre-equilibrated in Buffer B overnight at 4° C. The slurry was transferred to a gravity flow column, the flow-through and wash fractions were combined and assessed for the presence of soluble FcγR as described above. The combined fractions were concentrated and injected onto a Superdex 200 Hiload (GE Healthcare, Uppsala, Sweden) column equilibrated in 50 mM NaPO₄, 109 mM NaCl, pH 7.3 (Buffer C). The column was eluted in Buffer C and fractions containing soluble FcγR were combined, concentrated, sterile-filtered, and stored at −80° C. FcγRIIA-CH6 and FcγRIIIA-CH6 were analyzed by SDS-PAGE, Western blotting, N-terminal sequencing, and size exclusion multi-angle light scattering. Endotoxin levels were <1.0 endotoxin units/mL for each receptor preparation formulated at ˜20 mg/mL.

The nucleotide coding sequences for FcγRIIA-CH6 and FcγRIIIA-CH6 are shown in SEQ ID NO:32 and SEQ ID NO:34, respectively. The encoded polypeptide sequences for FcγRIIA-CH6 and FcγRIIIA-CH6 are shown in SEQ ID NO:33 and SEQ ID NO:35, respectively. N-terminal sequence analysis showed Gln-34 as the start site for mature FcγRIIA-CH6 and both Met-18 and Glu-21 as the start site for mature FcγRIIIA-CH6. Accordingly, the mature form the of FcγRIIA-CH6 polypeptide, without the signal sequence, corresponds to amino acid residues 34-222 of SEQ ID NO:33, while the mature forms of FcγRIIIA-CH6 correspond to amino acid residues 18-205 and 21-205 of SEQ ID NO:35.

### EXAMPLE 10

### Binding of Soluble His-Tagged FcγR (FcγRIA-CH6, FcγRIIA-CH6, and FcγRIIIA-CH6) to Immobilized Human IgG1

Measurements were performed using a Biacore 3000 instrument (Piscataway, N.J.). Activation of the sensor chip surface and covalent immobilization of the IgG1 antibody (Lambda from human myeloma plasma, Sigma-Aldrich, St. Louis, Mo.) was performed using 0.2 M N-ethyl-N′-(3-diethylamino-propyl) carbodiimide and 0.05 M N-hydroxysuccinamide and the Biacore Control Software. The human IgG1 antibody, diluted to 11 μg/mL in 10 mM sodium acetate, pH 5.0, was immobilized to prepare the specific binding flow cell, and a second flow cell was activated, but not exposed to IgG1 to prepare the reference flow cell. The un-reacted ester sites on both the specific binding and reference flow cells were blocked with 1 M ethanolamine hydrochloride.

For kinetic analysis of soluble FcγRIA binding, the IgG1 antibody was immobilized at a level of 458 resonance units (RU). FcγRIA-CH6 was injected over both the active and reference flow cells in series. For kinetic analysis of FcγRIA-CH6 binding, a concentration range of 0.16 to 10.3×10⁻⁹ M of FcγRIA-CH6 in HBS-EP (Biacore) assay buffer (10 mM Hepes, pH 7.4, 0.15M NaCl, 3.5 mM EDTA, 0.005% polysorbate 20) was used. FcγRIA-CH6 was injected at a flow rate of 40 μl/min for 3 minutes. Subsequently, the FcγRIA-CH6 solution was switched to HBS-EP buffer and dissociation was measured for 3 minutes. Each FcγRIA-CH6 concentration was tested in duplicate using a random sequence. Each measurement was followed by a single 30 second injection of 10 mM glycine-HCl, pH 1.8 at 50 μL/min to regenerate the IgG1 surface.

For equilibrium analyses of soluble FcγRIIA and FcγRIIIA binding, the IgG1 antibody was immobilized at a level of 1013 RU. A concentration range of 0.03-24×10⁻⁶ M of soluble FcγR was used. Each soluble FcγR (FcγRIIA-CH6 and FcγRIIIA-CH6) was injected at a flow rate of 10 μL/min for 1 minute. The dissociation time for each FcγR was 5 minutes. Each FcγRIIA-CH6 and FcγRIIIA-CH6 concentration was tested in duplicate using a random sequence. Each measurement was followed by a single 30 second injection of HBS-EP at 30 μL/min to regenerate the IgG1 surface.

Binding curves for all three soluble FcγRs were processed by subtraction of the reference surface curve from the specific binding surface curve, as well as subtraction of a buffer-injection curve. The processed binding curves were globally fitted to a 1:1 binding model and the resulting kinetic and equilibrium constants were evaluated using Biacore software.

The soluble FcγRs bound to immobilized human IgG1 in a manner that was best-fit by a 1:1 binding interaction. The IgG1 exhibited some loss of binding activity upon covalent immobilization and the activity of the surface ranged from 26-81% of the theoretical maximum. The association and dissociation phases of FcγRIA-CH6 binding to IgG1 were measurable over a time period of >200 seconds, allowing kinetic analysis of the binding curves. FcγRIA-CH6 bound to IgG1 with association (kₐ) and dissociation (k_{d}) rate constants of 2.8×10⁶ M⁻¹S⁻¹ and 4.6×10⁴ s⁻¹, respectively, yielding an equilibrium dissociation constant (K_{D}) of 1.7×10⁻¹⁰ M. The association/dissociation rates for FcγRIIA-CH6 and FcγRIIIA-CH6 were too fast to measure accurately, so the equilibrium dissociation constants were determined at steady state. Binding of FcγRIIIA-CH6 and FcγRIIA-CH6 to IgG1 was saturable and of low affinity with estimated K_{D}s of 0.63×10⁻⁶ M and 1.9×10⁻⁶ M, respectively. Each soluble FcγR bound to immobilized rabbit anti-OVA IgG with rates and affinities similar to that observed with human IgG1.

### EXAMPLE 11

### Purification of FcγRIA-Ig Fusion Protein

Protein A Affinity Purification: Delivered 0.22 μm filtered media assessed for expression of fusion protein using RP-HPLC and quantitative western blot probing against the heavy chain of the Ig tag. An appropriately sized Protein A column is constructed, assuming a binding capacity of no greater than 15 mg of fusion protein per mL of packed bed. The resin used can be either Poros A50 (AB Biosystems) or Recombinant Protein A Sepharose Fast Flow (GE Healthcare). The column is equilibrated in ZGI 1×PBS and the media is loaded over the column at 4 C, making sure not to exceed the maximum flow rate and pressure rating of the resin. Although the loading of the protein A resin can take place over multiple days, an overnight load is typical for 10-20L of delivered media. Once the load is complete, the column is washed with at least 10CV of ZGI 1×PBS, monitoring the absorbance at A280 nm and making sure that it is baseline stable for at least 10CV prior to elution. Elution of bound protein is accomplished via a pH shift using 0.1M Glycine at pH 3.0. Fractions are collected in the presence of enough 2M Tris pH 8.0 for immediate neutralization. Collected fractions are analyzed via RP-HPLC and SDS-PAGE and are pooled based upon the presence of Ig fusion protein.

If the Ig tag is based on a species other than human, then the appropriate resin to use (Protein A, G, or L) must be determined based upon the affinity of that species Ig subclass to the resin (literature). The capacity of Protein G (or L) Sepharose (GE Healthcare) for Ig fusions of species other than human would have to be determined empirically.

Downstream Processing: Normally, the affinity elution pool is pure enough to bypass any other conventional chromatographic techniques, but if it is not, further purification can be achieved via employing any one or a combination of a number of techniques. These techniques include, but are not limited to: anion exchange chromatography, cation exchange chromatography, hydrophobic interaction chromatography, lectin affinity chromatography, use of ceramic hydroxyapaptite, and heat aggregated IgG Sepharose affinity resin.

Size Exclusion Chromatography: The Protein A elution pool is concentrated at least 10-20× using an appropriate filtration set up. This set up can include, but is not limited to, the TFF labscale system equipped with 1×50 kD MWCO 0.1 cm² membrane (Millipore), a stirred cell system equipped with a YM30 membrane of appropriate diameter (Millipore), or a 30 kD MWCO Ultracel centrifugal membrane (Millipore). The volume of solution determines the set up used, with the above list going from large to small volume, respectively. The concentrate is injected over a Superdex 200 column (GE Healthcare) of appropriate size for the amount of volume and mass injected. Typically the volume injected does not exceed 7% of the column volume, and routinely multiple injections are performed to increase resolution. The column is equilibrated in formulation buffer, which is a phosphate buffered saline solution that is nearly isotonic. Solutions used have been: 50 mM NaPO₄, 109 mM NaCl, pH 7.3 and 35 mM NaPO₄, 120 mM NaCl pH 7.2. The column is eluted isocratically at a flow rate no greater than 45 cm/hr and fractions collected. Eluted fractions analyzed by SDS-PAGE and RP-HPLC and were pooled to yield the highest possible purity of Ig fusion protein. This step resolves the fusion protein from aggregates and any residual smaller molecular weight host cell contaminants.

Final Processing: Size exclusion pool concentrated to the appropriate concentration, which can be greater than 20 mg/mL. Concentration is performed using a stirred cell apparatus equipped with a YM30 membrane (30 kD MWCO). Final concentrate 0.22 μm filtered using a syringe filter (Millipore), aliquotted into the desired tubes (Starstedt) and stored at −80° C.

### EXAMPLE 12

### Construction of Mammalian Soluble FcγRIA Expression Constructs that Expresses Soluble Monomeric Untagged FcγRIA Protein

Two expression constructs containing the extracellular domain of human FcγRIA were constructed via PCR and homologous recombination using a DNA fragment encoding the extracellular domain of a short version FcγRIA (amino acids 1-282 of SEQ ID NO:2) and a long version FcγRIA (additional ten amino acids at the C-terminus) (amino acids 1-292 of SEQ ID NO:2) and the expression vector pZMP31.

PCR fragments encoding the short and long version of FcγRIA were constructed to contain a 5′ overlap with the pZMP31 vector sequence in the 5′ non-translated region, the FcγRIA extracellular domain coding region corresponding to SEQ ID NO:2 amino acid residues 1-282 or 1-292, respectively, and a 3′ overlap with the pZMP31 vector in the poliovirus internal ribosome entry site region. The PCR amplification reaction for both the short and long version used the 5′ oligonucleotide “zc57709” (ACTTTGCCTTTCTCTCCACAGGTGTCCAGGGAATTCATATAGGC CGGCCACCATGTGGTTCTTGACAACT; SEQ ID NO:36). The 3′ oligonucleotide “zc57710” (TGGGGTGGGTACAACCCCAGAGCTGTTTTAAGGCGCGCCTTTAGCCAAGCACTTGAAGC TCCA; SEQ ID NO:37) was used for the short version and the 3′ oligonucleotide “zc57712” (TGGGGTGGGTACAACCCCAGAGCTGTTTTAAGGCGCGCCTTTAATGAAACCAGACAGGA GT; SEQ ID NO:38) was used for the long version. The FcγRIA template was from a previously generated cDNA of FcγRIA.

The PCR amplification reaction conditions were as follows: 1 cycle, 95° C., 2 minutes; 30 cycles, 95° C., 15 seconds, followed by 55° C., 30 seconds, followed by 68° C., 1 minute. The PCR reaction mixture was run on a 1% agarose gel and the DNA fragment corresponding to the expected size was extracted from the gel using a GE Healthcare illustra GFX™ PCR DNA and Gel Band Purification Kit.

Plasmid pZMP31 is a mammalian expression vector containing an expression cassette having the chimeric CMV enhancer/MPSV promoter, an EcoRI site for linearization prior to yeast recombination, an internal ribosome entry element from poliovirus; an E. coli origin of replication and ampicillin selectable marker; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP31 was digested with EcoRI prior to recombination in yeast with each of the gel extracted FcγRIA PCR fragments of the short and long version. One hundred μl of competent yeast (S. cerevisiae) cells were combined with 20 μl of the FcγRIA short or long insert DNA and ˜100 ng of EcoRI digested pZMP31 vector. The mix was transferred to a 0.2 cm electroporation cuvette. The yeast/DNA mixture was electropulsed using power supply (BioRad Laboratories, Hercules, Calif.) settings of 0.75 kV (5 kV/cm), ∞ ohms, and 25 RF. Six hundred μl of 1.2 M sorbitol was added to the cuvette, and the yeast was plated in two 300 μl aliquots onto two URA-D plates and incubated at 30° C. After about 72 hours, the Ura+ yeast transformants from a single plate were resuspended in 800 μl H₂O and spun briefly to pellet the yeast cells. The cell pellet was resuspended in 0.5 ml of lysis buffer (2% Triton X-100, 1% SDS, 100 mM NaCl, 10 mM Tris, pH 8.0, 1 mM EDTA). The five hundred μl of the lysis mixture was added to an Eppendorf tube containing 250 μl acid-washed glass beads and 300 μl phenol-chloroform, was vortexed for 3 minutes, and spun for 5 minutes in an Eppendorf centrifuge at maximum speed. Three hundred μl of the aqueous phase was transferred to a fresh tube, and the DNA was precipitated with 600 μl ethanol, followed by centrifugation for 10 minutes at maximum speed. The tube was decanted and the pellet was washed with 1 mL of 70% ethanol, followed by centrifugation for 10 minutes at maximum speed. The tube was decanted and the DNA pellet was resuspended in 10 μl H₂O.

Transformation of electrocompetent E. coli host cells (DH10B) was done using 1 μl of the yeast DNA preparation and 20 μl of E. coli cells. The cells were electropulsed at 2.0 kV, 25 μF, and 400 ohms. Following electroporation, 600 μl SOC (2% Bacto™ Tryptone (Difco, Detroit, Mich.), 0.5% yeast extract (Difco), 10 mM NaCl, 2.5 mM KCl, 10 mM MgCl₂, 10 mM MgSO₄, 20 mM glucose) was added and the cells were plated in 50 μl and 550 μl aliquots on two LB AMP plates (LB broth (Lennox), 1.8% Bacto™ Agar (Difco), 100 mg/L Ampicillin).

The colonies were screened via colony PCR and the inserts of five DNA clones from each construct were subjected to sequence analysis. One clone containing the correct sequence was selected. DNA sequencing was performed using ABI PRISM BigDye Terminator v3.1 Cycle Sequencing Kit (Applied Biosystems, Foster City, Calif.). Sequencing reactions were purified using EdgeBioSystems Preforma Centriflex Gel Filtration Cartridges (Gaithersburg, Md.) and run on an Applied Biosystems 3730 DNA Analyzer (Applied Biosystems, Foster City, Calif.). Resultant sequence data was assembled and edited using Sequencher v4.6 software (GeneCodes Corporation, Ann Arbor, Mich.). One clone containing the correct sequence was selected and large-scale plasmid DNA was isolated using a commercially available kit (QIAGEN Plasmid Mega Kit, Qiagen, Valencia, Calif.) according to manufacturer's instructions.

The sequences of the short and long versions of the insert DNA are shown in SEQ ID NO:39 and SEQ ID NO:41, respectively. The corresponding encoded amino acid sequences for the short and long versions of untagged FcγRIA are shown in SEQ ID NO:40 and SEQ ID NO:42, respectively. The signal sequence for FcγRIA corresponds to amino acids 1-15 of SEQ ID NO:2 (residues 1-15 of SEQ ID NOs 40 and 42), thereby yielding a start site for the mature untagged FcγRIA proteins at position 16 of SEQ ID NOs 40 and 42.

### EXAMPLE 13

### Transfection and Expression of Soluble FcγRIA Receptor Expression Constructs that Express Untagged FcγRIA Protein

Two hundred μg of the soluble FcγRIA short and long version expression constructs were digested with 200 units of BstB1 at 37° C. for eighteen hours (overnight), washed with phenol/chloroform/isoamyl alcohol, precipitated with ethanol, and centrifuged in a 1.5 mL microfuge tube. The supernatant was decanted off the pellet, and the pellet was washed with 1 mL of 70% ethanol and allowed to incubate for 5 minutes at room temperature. The tube was spun in a microfuge for 10 minutes at 14,000 RPM and the supernatant was decanted off the pellet. The pellet was then resuspended in 200 μl of CHO cell tissue culture medium in a sterile environment, allowed to incubate at 37° C. for 30 minutes. Approximately 1×10⁷ CHO cells were pelleted and were resuspended using the DNA-medium solution. The DNA/cell mixtures were placed in a 0.4 cm gap cuvette and electroporated using the following parameters; 950 μF, high capacitance, at 300 V. The contents of the cuvettes were then removed, pooled, and diluted to 25 mL with CHO cell tissue culture medium and placed in a 125 mL shake flask. The flask was placed in an incubator on a shaker at 37° C., 5% CO₂ with shaking at 120 RPM.

The CHO cells were subjected to nutrient selection and amplification to 200 nM Methotrexate (MTX). Tagged protein expression was confirmed by Western blot, and the CHO cell pool was scaled-up for harvests for protein purification.

### EXAMPLE 14

### Purification of Untagged FcγRIA

The following method applies to the purification of untagged FcγR1a from CHO DXB11 cell conditioned media.

### A. IgG Affinity Chromatography

Undiluted (1×) media were harvested and were loaded over a column containing IgG Sepharose 6 Fast Flow resin (GE Healthcare) at a flow rate of 15 cm/h. For 10 L of conditioned media, a 5 cm diameter column containing 150 mL of packed resin was employed. After loading the media, the column was washed at 100 cm/h with 1.6 mM citric acid, 23 mM dibasic NaPO₄, 150 mM NaCl pH 7.0 until the absorbance at 215 nm and A280 nm returned to baseline for at least 2 column volumes (CV). Elution of bound protein was achieved using a 10 CV descending pH gradient of 20 mM citric acid, 5 mM dibasic NaPO₄, 0.05% Tween 20, pH 3.0 at a flow rate of 61 cm/h. Fractions containing FcγR1A were identified by SDS-PAGE and Western blotting, were neutralized by the addition of 2 M Tris pH 7.0 to a 0.2 M final concentration and brought to 100 mM NaCl by the addition of 4 M NaCl.

### B. Cation Exchange Chromatography

The Tween-20 was removed from the FcγR1A pool by HS50 chromatography. The FcγR1A elution pool was adjusted to 10 mM MES pH 6.0 using solid MES and HCl and was diluted to <5 mS/cm using 10 mM MES pH 6.0. The FcγR1A-containing pool was loaded over an HS50 column to achieve quantitative capture at a flow rate of 141 cm/h and the resin was washed at 382 cm/h with 10 mM MES pH 6.0 until A215 and A280 nm UV signals returned to baseline for at least 5 CV. Bound FcγR1A was eluted at 382 cm/hr with a gradient of increasing NaCl concentration using 5 CV to a maximum of 60% elution buffer which consisted of 10 mM MES, 2 M NaCl pH 6.0. Fractions were collected and FcγR1A was identified by SDS-PAGE and Western blotting.

### C. Size Exclusion Chromatography

The amount of protein as assessed by absorbance at 280 nm and the FcγR1A-containing fraction of the buffer-exchanged HS50 elution pool was concentrated using a 30 kD molecular weight cutoff (MWCO) Ultracel centrifugal concentrator or a YM30 63.5 mm stirred cell membrane depending on the amount of FcγR1A present. The final concentrate volume was no more than 3% of the volume of gel filtration column used. The concentrated FcγR1A pool was injected onto a Superdex 75 column (for <1 mg FcγR1A, the column size was 10/300 mm; for 1-10 mg, the column size was 16/60 mm; and for >10 mg, the column size was 26/60 mm) and the protein was eluted isocratically at a flow rate of 34-76 cm/h. The mobile phase used was 35 mM NaPO₄, 120 mM NaCl pH 7.2. Fractions were collected and FcγR1A was identified by SDS-PAGE and Western blotting. The FcγR1A-containing fractions were concentrated to 20 mg/mL final concentration as described above, passed through a 0.22 μm sterile-filter, and stored at −80° C. The identity of the FcγR1A was confirmed by N-terminal sequencing and amino acid analyses. N-terminal sequence analysis showed that the mature protein starts with a pyro-glutamic acid, which is post-translationally converted from the glutamine residue at amino acid position 16.

### EXAMPLE 15

### Anti-Inflammatory Activities of Soluble FcγRIA

### A. Immune Complex Precipitation

Chicken egg ovalbumin (OVA) was dissolved to a final concentration of 15.0 μg/mL in phosphate buffered saline (PBS) and combined with 300 μg rabbit polyclonal anti-OVA antibodies/mL in a final volume of 200 μl in the presence and absence of the indicated concentration of soluble FcγRIA. Immediately thereafter, turbidity of the reaction mixture was monitored at 350 nm every 30 seconds for 5-10 min at 37° C. with the aid of a spectrophotometer. Linear regression was used to calculate the slope of the linear portion of the turbidity curves and the FcγR-mediated inhibition of immune complex precipitation was expressed relative to incubations containing anti-OVA and OVA alone.

B. Cytokine Secretion from Mast Cells

Immune complexes were prepared by mixing 300 uL of rabbit polyclonal anti-OVA with 75.0 μl of 1 mg OVA/mL in PBS in a final volume of 5.0 mL of PBS. After incubation at 37° C. for 30-60′, the mixture was placed at 4° C. for 18-20 h. The immune complexes were collected by centrifugation at 12,000 rpm for 5.0 min, the supernatant fraction was removed and discarded, and the immune complex precipitate was resuspended 1.0 mL of ice cold PBS. After another wash, the immune complexes were resuspended in a final volume of 1.0 mL ice cold PBS. Protein concentration was determined using the BCA assay.

MC/9 cells were sub-cultured in Medium A (DMEM containing 10% fetal bovine serum, 50.0 μM B-mercaptoethanol, 0.1 mM non-essential amino acids, 1.0 mM sodium pyruvate, 36.0 μg/mL L-asparagine, 1.0 ng/mL rmIL-3, 5.0 ng/mL rmIL-4, 25.0 ng/mL rmSCF) to a density of 0.5-3×10⁶ cells/mL. Cells were collected by centrifugation at 1500 rpm for 5.0 min and the cell pellet was washed in Medium A (without cytokines) and resuspended in Medium A at 2.0×10⁶ cells/mL. Aliquots of cells (2.0×10⁵ cells) were incubated with 10.0 μg/well of OVA/anti-OVA immune complexes (IC's) in a final volume of 200 μl of Buffer A in a 96-well microtiter plate. After 4.0 h at 37° C., the media was removed and centrifuged at 1500 rpm for 5.0 min. The cell-free supernatant fractions were collected and aliquots were analyzed for the presence of IL-6, IL-13, TNFα, and MCP-1 cytokine release using a Luminex cytokine assay kit.

### C. Complement-Mediated Lysis of SRBCs

Antibody-sensitized SRBCs (Sigma-Aldrich, St. Louis, Mo.) were prepared and were incubated with different concentrations of soluble FcγRIA. After 15 minutes at 4° C., a 25 μl sample of a 1:50 dilution of rat serum (Sigma-Aldrich, St. Louis, Mo.) was added, and hemolysis was measured by monitoring the absorbance of the mixture at 540 nm as described by the manufacturer.

### D. Measurement of FcγRIA-CH6 Affinity for Human IgG1

The IgG1 antibody was immobilized to a single flow cell, utilizing a second non-derivatized cell as the blank reference. Immobilization of the IgG1 antibody was performed using an amine coupling kit (Biacore) and the standard Wizard Template for Surface Preparation, operated by the Biacore Control Software. Based on Wizard results for a pH scouting study, the IgG1 antibody solution was diluted to 11 μg/mL in sodium acetate, pH 5.0. The Wizard Template for amine coupling was used to immobilize the antibody to a single flow cell. The carboxyl groups on the sensor surfaces were then activated with an injection of a solution containing 0.2 M N-ethyl-N′-(3-diethylamino-propyl)carbodiimide (EDC) and 0.05 M N-hydroxysuccinimide (NHS). The antibody solution was then injected over the activated surface targeting a level of 150-200 RU. The immobilization procedure was completed by blocking remaining ester sites on the carboxymethyl dextran surface with 1 M ethanolamine hydrochloride.

The method for injection of the analyte solutions (FcγRIA-CH6) was written using the Biacore Wizard Template for kinetic analysis. The method was run at 25° C. and the samples stored in the autosampler at ambient temperature. It is noted that in using the Wizard Template, certain parameters optimal for kinetics, such as injection modes, are pre-defined by the Wizard program.

The method for analysis of FcγRIA was optimized for determination of kinetic rate constants, kₐ and k_{d}. The receptor was injected over both flow cells (i.e., 1 and 2, blank and antibody-derivatized, respectively) in series to allow for comparative analysis of binding of the FcγRIA to the human IgG1 antibody vs. binding of the FcγRIA to the non-modified control surface (binding to rabbit anti-OVA IgG not tested). The analyte was injected at a flow rate of 40 μl/min for 3 minutes (association time). The dissociation time for each analyte injection was 3 minutes. The analyte dose response curve range was 0.16-10.3 nM. For each dose response curve point, N=2 replicate injections were run. The sequence included injections of buffer for subtraction of instrument noise and drift. Dose response curve samples were injected in random mode. For kinetic analysis of FcγRIA, each dose response curve cycle was followed by a single 30 second injection of glycine, pH 1.75 at 50 μl/minute to regenerate the IgG antibody surface.

Data analysis was performed using Biacore Control, Evaluation and Simulation software. Baseline stability was first assessed to ensure that the regeneration step provided a consistent binding surface throughout the sequence of injections. The level of non-specific binding of the FcγRIA analyte to the control surface was checked and confirmed to be minimal. Binding curves were processed by subtraction of the control surface curve (i.e., flow cell 1) from the specific binding surface curve (i.e., flow cell 2), as well as subtraction of instrument noise and drift using a buffer injection curve. The data was checked for reproducibility of analyte injections and the resulting corrected binding curves were then globally fitted to binding models and the resulting fit and equilibrium constants evaluated.

### E. Cutaneous Reverse Passive Arthus Reaction in Mice

Ten-week old female C57BL/6 mice (n=8 mice per group) were anesthetized with isoflurane, their dorsal skin was shaved, and the back of each mouse was wiped with 70% alcohol. Each mouse received two intradermal injections of 0.02 mL each, at distinct sites in the dorsal skin. The injection solutions contained phosphate buffered saline (PBS) and either 40.0 μg of rabbit anti-ovalbumin (anti-OVA, heat-inactivated by incubation at 56° C. for 30-40 min) alone or 40.0 μg of anti-OVA and the indicated amount of FcγRIA-CH6. Mice in the control groups received two intradermal injections of 40.0 μg non-immune rabbit IgG (heat-inactivated as described above). Antibody preparations were centrifuged at 14,000 rpm for 10 min to remove particulates prior to injection. Immediately following the intradermal injections, each mouse was injected in the tail vein with 100.0 μl of a solution containing 10.0 mg OVA/mL and 10.0 mg Evan's Blue/mL. In some instances, the tail vein injection solution also contained dexamethazone at a dose of 1.0 mg/kg. Four hours after the injections, the mice were euthanized by CO₂ gas. Cutaneous edema was evaluated by measuring the area of vascular leak of Evan's Blue dye (mm²) and by measuring tissue weights (mg) of punch biopsies taken from the lesion sites. The tissue samples were then quickly frozen in liquid N₂ and stored at −80° C.

Neutrophil infiltration was assessed by measuring myeloperoxidase activity in the punch biopsy samples as described (Bradley et al., J. Invest. Dermatol. 78:206-209, 1982) using the Myeloperoxidase Assay Kit from Cytostore (Calgary, Alberta Canada).

Systemic administration of FcγRIA-CH6 in mice was performed by intravenous injection of either vehicle alone or vehicle containing the indicated concentrations of FcγRIA-CH6. Each mouse received the indicated dose of FcγRIA-CH6 in a 0.1 mL final volume of formulation buffer (35 mM sodium phosphate, 120 mM NaCl, pH 7.2) 1.0-h prior to initiating the Arthus reaction. The cutaneous Arthus reaction in mice was performed exactly as described above.

### F. Results and Discussion

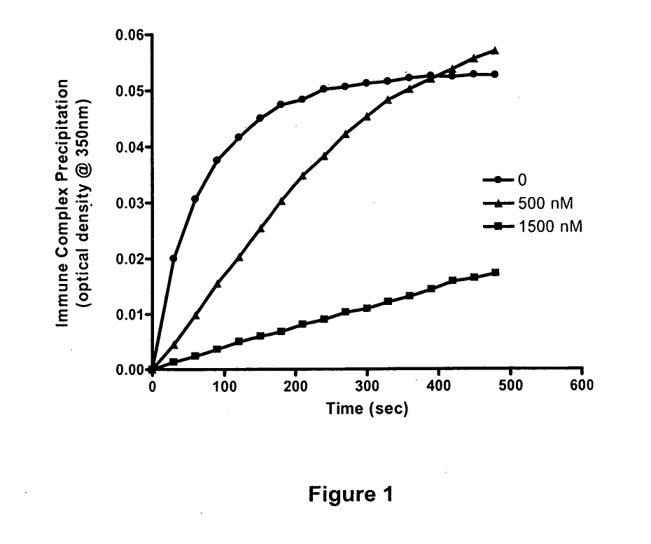

To evaluate whether FcγRIA-CH6 could block immune complex precipitation, an anti-OVA/OVA immune complex precipitation assay was established based on the methods of MØller (Immunology 38:631-640, 1979) and Gavin et al. (Clin. Exp. Immunol. 102:620-625, 1995). Incubation of anti-OVA and OVA at 37° C. produced a time-dependent increase in optical density of the solution mixture (FIG. 1, circles), an observation consistent with the formation of insoluble anti-OVA/OVA immune complexes. Addition of FcγRIA-CH6 at the start of the assay produced a dose-dependent reduction in immune complex precipitation (FIG. 1, triangles and squares). Immune complex precipitation was completely abolished by 1500 nM FcγRIA-CH6. Identical data were obtained when untagged, recombinant soluble FcγRIA was used. Since the precipitation of antigen:antibody immune complexes appears to be dependent on non-covalent interactions between the antibody Fc heavy chains (MØller, Immunology 38:631-640) and Fcγ receptors bind to the Fc portion of antibodies (Dijstelbloem H M et al., Trends Immunol. 22:510-516, 2001), these data suggest that soluble FcγRIA disrupts immune complex precipitation by binding to the Fc portion of the anti-OVA antibodies.

To directly evaluate the interaction of FcγRIA-CH6 with antibody Fc, the binding of FcγRIA-CH6 to immobilized human IgG1 was assessed by surface plasmon resonance analyses. A monoclonal human IgG1 antibody was immobilized to the sensor surface in a single flow cell at an RU (resonance units) level of 485, a density level within optimal levels for kinetic analysis of FcγRIA-CH6, presuming a binding stoichiometry of one FcγRIA molecule with one IgG1 molecule (Woof and Burton, Nature Rev. Immunol. 4:1-11, 2004). FcγRIA rapidly bound to immobilized IgG1 with rates of association and dissociation of 2.8×10⁶ M⁻¹s⁻¹ and 4.6×10⁻⁴ s⁻¹, respectively, values which yield a calculated equilibrium dissociation constant of 1.7×10⁻¹⁰ M. These data are similar to those reported previously (Paetz A et al., Biochem. Biophys. Res. Commun. 338:1811-1817, 2005) and demonstrate that FcγRIA-CH6 binds with high affinity to human IgG1.

Mast cells are thought to mediate immune complex-mediated inflammation in a variety of immune disorders such as type III hypersensitivity reactions (Ravetch, J. Clin. Invest. 110:1759-1761, 2002; Sylvestre and Ravetch, Immunity 5:387-390, 1996; Jancar and Crespo, Trends Immunology 26:48-55, 2005). Binding of immune complexes to mast cell Fcγ receptors is thought to induce the secretion of pro-inflammatory cytokines, such as IL-6 and TNFα (Ravetch, supra; Jancar and Crespo, supra), which subsequently leads to neutrophil infiltration and tissue damage. To evaluate whether cytokine secretion from mast cells could be stimulated by immune complexes, the murine mast cell line MC/9 was incubated in the presence and absence of preformed rabbit anti-OVA/OVA immune complexes. Incubation with anti-OVA/OVA immune complexes produced a time and concentration dependent increase in the accumulation of the inflammatory cytokines IL-6, IL-13, TNFα, and MCP-1 within the MC/9 cell conditioned media. Cytokine production was not altered, in contrast, when MC/9 cells were incubated with an equivalent concentration of rabbit anti-OVA IgG alone. These data demonstrate that MC/9 cells respond to immune complexes by the production of inflammatory cytokines.

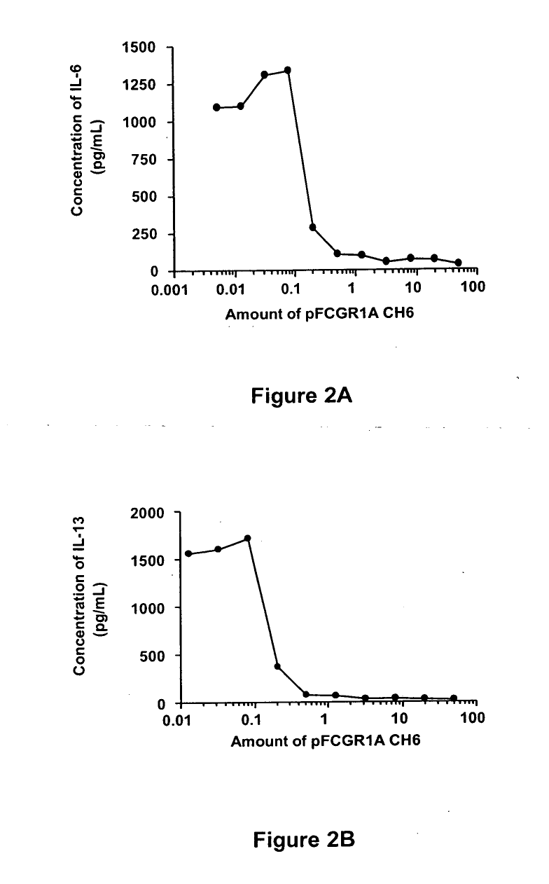

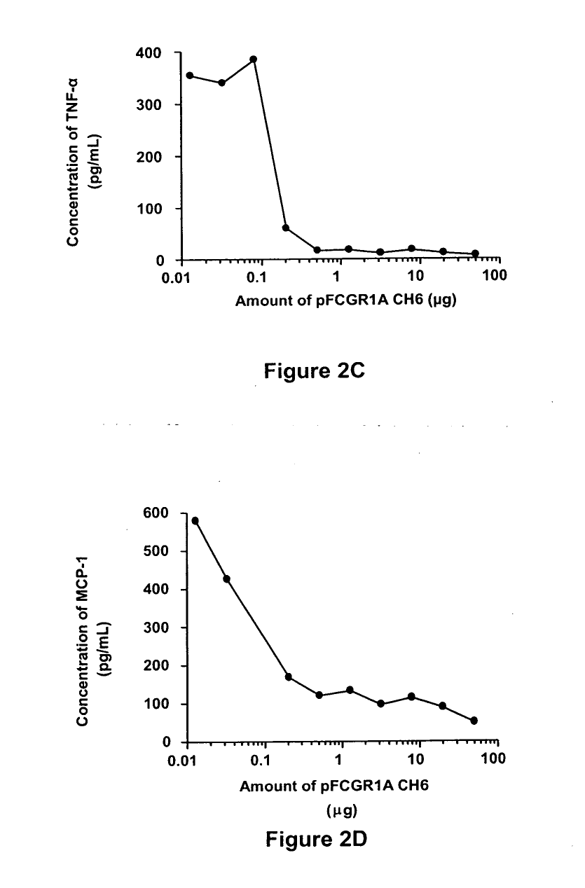

Incubation of MC/9 cells with anti-OVA/OVA immune complexes in the presence of increasing amounts of FcγRIA-CH6 produced dose-dependent reductions in the accumulation of IL-6 (FIG. 2A), IL-13 (FIG. 2B), TNFα (FIG. 2C) and MCP-1 (FIG. 2D). Identical data were obtained when untagged, recombinant soluble FcγRIA was used. These data demonstrate that soluble FcγRIA can block the binding and signalling of immune complexes in mouse mast cells.

Soluble FcγRIA was also evaluated for its effect on complement-mediated lysis of antibody-sensitized SRBCs. Incubation of antibody-sensitized SRBCs with rat serum at 37° C. resulted in complement activation and lysis of the SRBCs. Addition of FcγRIA-CH6 to the incubation mixtures blocked SRBC lysis in a dose-dependent manner. Little or no inhibition of hemolysis was observed, in contrast, with an unrelated control protein, TACI-Ig.

The findings described above demonstrate that FcγRIA-CH6 can block the formation of immune complexes in vitro, can inhibit immune complex-mediated signalling in mast cells, and can block IgG-mediated complement activity. These data suggest that FcγRIA-CH6 may be effective at blocking IgG- or immune complex-mediated inflammation in an in vivo setting. To test this, the cutaneous reversed passive Arthus reaction was established in mice and the effects of FcγRIA-CH6 on immune complex-mediated edema and neutrophil infiltration were assessed.

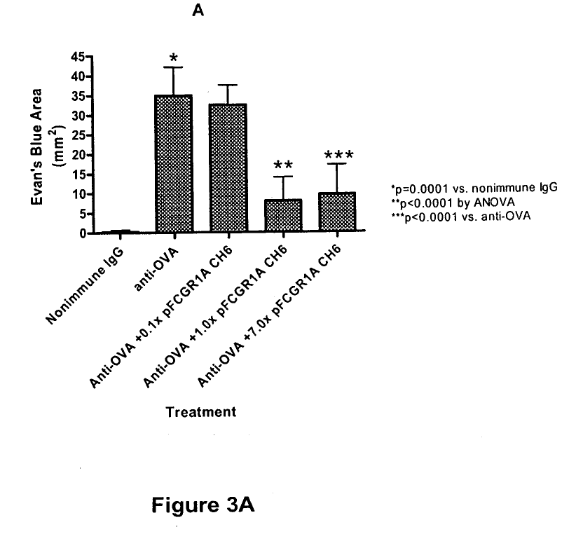

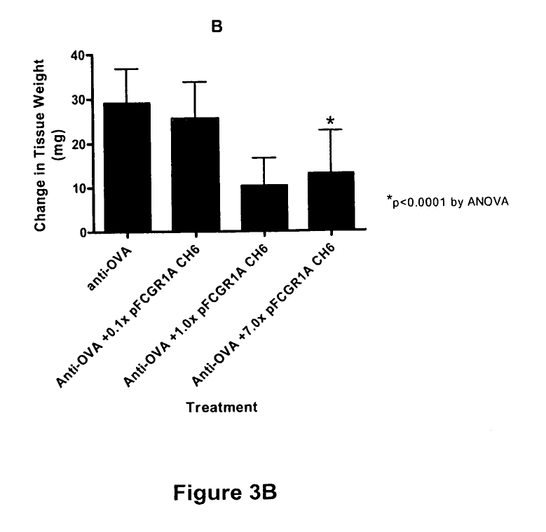

Relative to intradermal injection of an equivalent concentration of nonimmune IgG, injection of anti-OVA antibodies produced a time and concentration increase in edema within the skin of treated mice. Edema was evident as both an increase in the area of extravasation of Evan's blue dye (FIG. 3A) and in tissue weights (FIG. 3B). These effects were specific for immune complexes as no edema was observed in the absence of tail vein injection of OVA. Accumulation of neutrophils within the lesion site, measured by extractable activity of myeloperoxidase, was also increased (FIG. 3C).

Intradermal delivery of anti-OVA antibodies with increasing amounts of FcγRIA-CH6 produced a concentration-dependent reduction in edema, measured by either a decrease in Evan's blue area (FIG. 3A) or a decrease in tissue weight of the lesion site (FIG. 3B). Myeloperoxidase activity in the lesion biopsies was also significantly decreased by FcγRIA-CH6 (FIG. 3C). These data demonstrate that FcγRIA-CH6 was an effective inhibitor of immune complex-induced inflammation in the Arthus reaction in mice.

These data demonstrate that local delivery of FcγRIA-CH6 can block immune complex-mediated dermal inflammation in the Arthus reaction in mice.

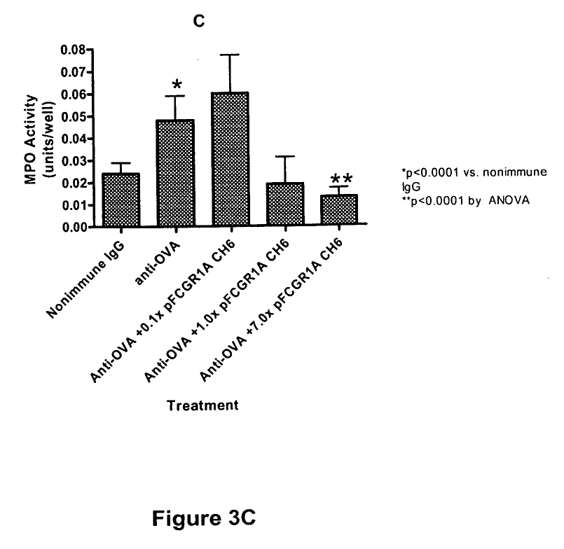

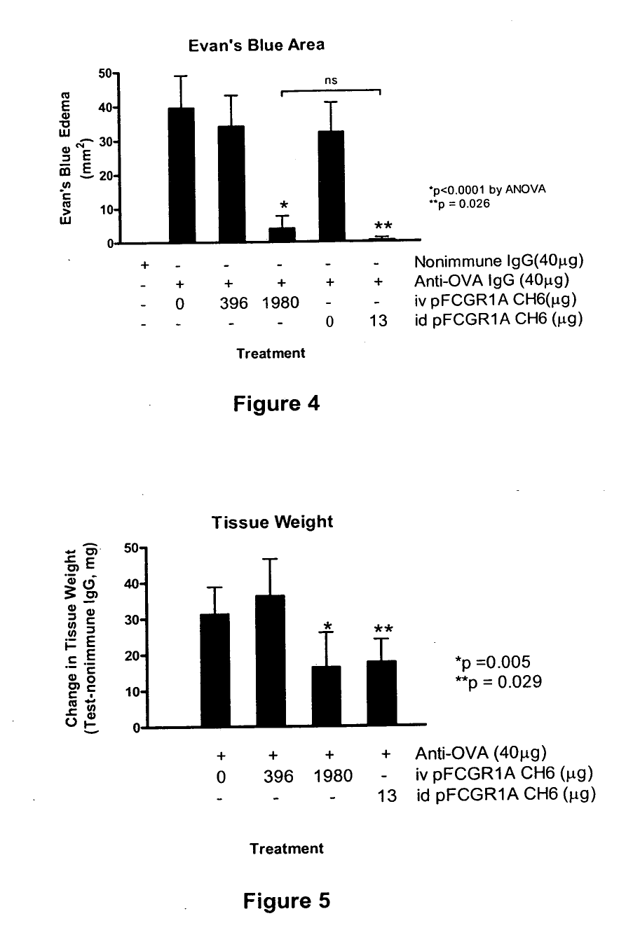

To evaluate whether systemic delivery of FcγRIA-CH6 could reduce cutaneous inflammation, mice were injected with FcγRIA-CH6 via the tail vein, 1.0-h prior to initiating the Arthus reaction. Compared to injection with vehicle alone, injection with FcγRIA-CH6 produced dose-dependent reductions in edema, measured either by the anti-OVA induced extravasation of Evan's Blue dye (FIG. 4) or by the anti-OVA induced increases in tissue weights of the lesion sites (FIG. 5). With the highest dose of FcγRIA-CH6, edema was virtually abolished (FIGS. 4 and 5). Similar to the data described above, intradermal delivery of 13.0 μg of FcγRIA-CH6 also reduced edema in this model (FIGS. 4 and 5). The reduction in edema seen with the highest dose of FcγRIA-CH6 given by the intravenous route was similar to that observed with intradermal delivery of 13.0 μg of FcγRIA-CH6 (FIGS. 4 and 5). Accumulation of neutrophils within the lesion sites, measured by extractable myeloperoxidase activity was also abolished in animals treated with FcγRIA-CH6.

### EXAMPLE 16

### Comparison of the Anti-Inflammatory Activities of Recombinant Human FcγRIA, FcγRIIA, and FcγRIIIA

In addition to the evaluation of monomeric FcγRIA-CH6 for anti-inflammatory activities (see Example 15, supra), monomeric FcγRIIA-CH6 and FcγRIIIA-CH6 (prepared as described above in Example 9) were also tested using the same in vitro and in vivo assays described in Example 15. Soluble FcγRIIA-CH6 and FcγRIIIA-CH6 were tested in parallel with FcγRIA-CH6 for their effects on immune complex precipitation, cytokine secretion from mast cells, and IgG-mediated complement activity. Similar to FcγRIA-CH6, both FcγRIIA-CH6 and FcγRIIIA-CH6 reduced immune complex precipitation, blocked complement-mediated lysis of antibody-sensitized red blood cells, and inhibited immune complex-mediated accumulations of IL-6, IL-13, MCP-1 and TNF-α in mast cell-conditioned media. The relative order of potency with respect to the reduction in immune complex precipitation was FcγRIIIA>FcγRIA>FcγRIIA, with maximal inhibition seen using 1-1.5 μM for each soluble FcγR, a molar ratio of FcγR:anti-OVA of approximately 1:1. The relative order of potency for both the blockade of complement-mediated lysis and inhibition of mast cell cytokine secretion was FcγRIA>FcγRIIIA>FcγRIIA. With respect to inhibition of cytokine secretion, for each soluble FcγR, the IC₅₀s were similar for each cytokine examined.

FcγRIIA-CH6 and FcγRIIIA-CH6 were also tested in parallel with FcγRIA-CH6 for their effects in vivo on edema and neutrophil infiltration in the cutaneous Arthus reaction in mice. In contrast to the reduction in inflammation observed with soluble FcγRIA-CH6, neither FcγRIIIA-CH6 nor FcγRIIA-CH6, used over a similar concentration range, reduced anti-OVA induced extravasation of Evan's blue dye, tissue weight, or tissue MPO activity (see FIG. 6, A-C).

Dimeric Fc5 fusion protein versions of FcγRIIA and FcγRIIIA, each containing two molecules of the extracellular domains of FcγRIIA or FcγRIIIA fused to an effector negative version of human Fc (Fc5), were also prepared and tested in the assays described above. The nucleotide and encoded amino acid sequences for FcγRIIA-Fc5 are shown in SEQ ID NO:43 and SEQ ID NO:44, respectively, while the nucleotide and encoded amino acid sequences for FcγRIIIA-Fc5 are shown in SEQ ID NO:45 and SEQ ID NO:46, respectively. N-terminal sequence analysis showed Gln-34 as the start site for mature FcγRIIA-Fc5 and Met-18 and Glu-21 as the start site for mature FcγRIIIA-Fc5. Each of the dimeric Fc5 fusion proteins had activities similar to the monomeric versions of each protein in all of the in vitro assays described above. Similar to their monomeric counterparts, and again in contrast to FcγRIA-CH6, neither FcγRIIA-Fc5 nor FcγRIIIA-Fc5 reduced inflammation or neutrophil infiltration in the reverse passive Arthus reaction in mice.

### EXAMPLE 17

### Collagen Antibody-Induced Model of Arthritis

Male DBA/1J mice (8 weeks old, n=8 mice per group) were administered 2 mg (in 200 uL) of the anti-Type II collagen antibody cocktail (Chemicon Intl. Arthrogen-CIA®) via intravenous tail injection on Day 0. The amount of mAb cocktail injected was based on literature values and on data from preliminary studies where 2.0 mg doses of Arthrogen-CIA® gave clear and consistent symptoms of arthritis in male DBA/1 mice. Three days later, mice received sub-cutaneous injections of either vehicle alone (PBS) or vehicle containing the indicated concentration (0, 0.67, or 2.0 mg) of FcγRIA-CH6. Three and one-half hours later, all mice received an intraperitoneal injection of 50 ug of LPS dissolved in a final volume of 50 uL of PBS, as provided in the Arthrogen kit. Mice were treated with vehicle or the indicated concentration of FcγRIA-CH6 every other day for a total of five doses.

Mice were scored (visual scores and caliper paw measurements) for arthritis on a daily basis starting on day 0, prior to injection of the Arthrogen-CIA® antibody cocktail. Mice were be sacrificed on day 11. Serum was collected and frozen at −80° C. Paws were collected into 10% NBF, and processed for histology.

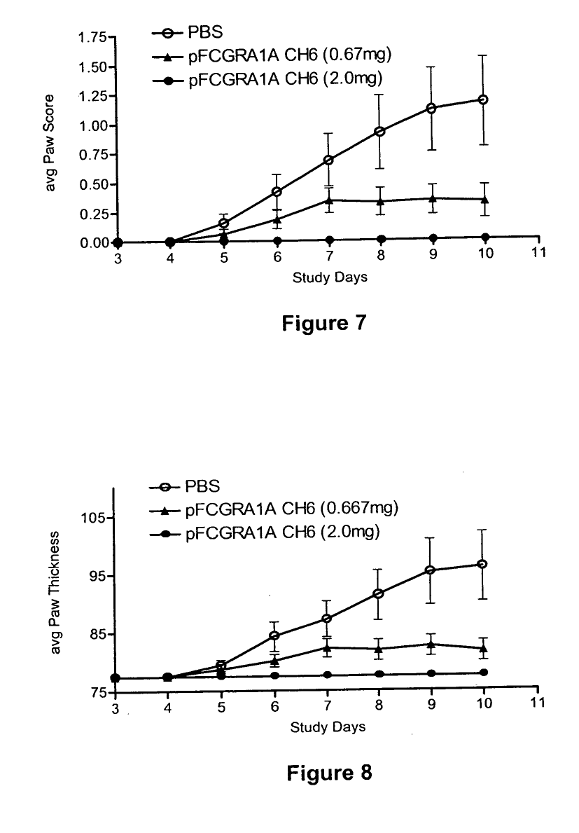

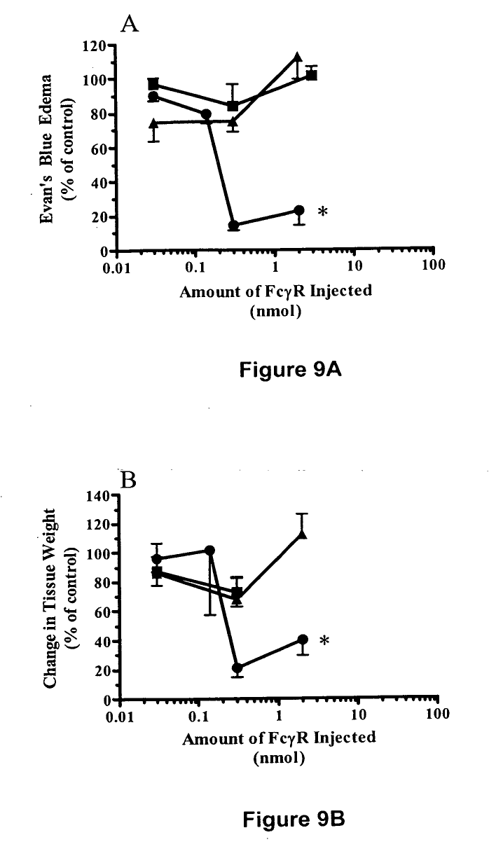

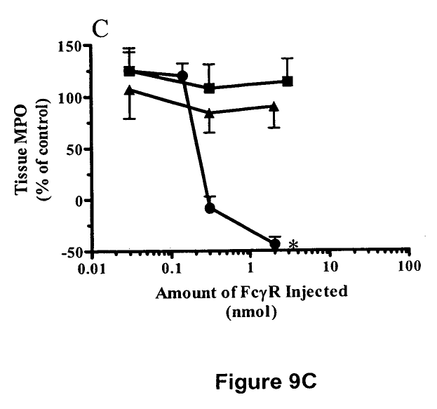

Treatment of mice with the Arthrogen-CIA® antibody cocktail, produced a time-dependent increase in paw inflammation, measured by either the visual paw score (FIG. 7, PBS treated) or by paw thickness (FIG. 8, PBS treated). The increase in arthritis score is easily observed in animals treated with vehicle alone (PBS). Treatment of animals with FcγRIA-CH6 produced a concentration-dependent reduction in paw inflammation. Antibody-induced inflammation, evident as the visual paw score (FIG. 7) or paw thickness (FIG. 8), was completely abolished by the highest dose of FcγRIA-CH6 administered. A less robust reduction in these parameters was seen with the 0.67 mg dose of FcγRIA-CH6 administered. These data demonstrate that FcγRIA-CH6 has potent anti-inflammatory properties in a setting of arthritis.

### EXAMPLE 18

### Treatment of Cryoglobulinemia with FcγRIA in TSLP Transgenic Mice

Mice over-expressing thymic stromal lymphopoietin (TSLP), an interleukin-7 (IL-7)-like cytokine with B-cell promoting properties, produce large amounts of circulating cryoglobulins of mixed IgG-IgM composition. (See Taneda et al., Am. J. Pathol. 159:2355-2369, 2001.) Development of mixed cryoglobulinemia in these animals is associated with systemic inflammatory disease involving kidneys, liver, lungs, spleen, and skin (see id.) due to immune complex deposition in these tissues. Kidney disease in these animals closely resembles human cryoglobulinemia glomerulonephritis as seen in patients with HCV infection. A role for Fcγ receptors in the disease process was shown by the exacerbation of renal injury with accelerated morbidity and mortality after deletion of the inhibitory receptor Fcγ receptor IIb (see Muhlfeld et al., Am. J. Pathol. 163:1127-1136, 2003). In view of these data, the studies described herein, demonstrating efficacy of soluble FcγRIA against immune-complex-mediated inflammation, suggest that TSLP-transgenic mice are a suitable model for evaluating efficacy of soluble FcγRIA for treating cryoglobulinemia.

Groups of ten TSLP-transgenic mice (three to six weeks of age) are treated with either vehicle alone, or vehicle containing 0.1, 0.3, 0.9, or 2.0 mg of soluble recombinant human FcγRIA by subcutaneous injections. Animals are dosed with either vehicle or vehicle with FcγRIA by a variety of dosing schedules (e.g., every other day over 21 days or every fourth day over 21 days).

At 21 days following dosing, a urine sample is collected for measurement of albuminuria, the animals are anesthetized with halothane, and blood is drawn by cardiac puncture. Spleen, kidneys, liver, ears, and lungs are removed and routinely processed for histology. For all organs, 4 μm sections from formalin-fixed and paraffin-embedded tissue are stained with hematoxylin and eosin (H&E) following routine protocols. From the kidney, 2 μm sections are stained with H&E, periodic acid Schiff reagent (PAS), and periodic acid methenamine silver stain.

Blood urea nitrogen is measured using a standard clinical chemistry analyzer and serum stored at 4° C. is assessed for the presence of cryoglobulins by visual inspection. Urine albumin to creatinine ratio is calculated to evaluate albuminuria by standard procedures.

Morphometry is performed on H&E-stained and silver-stained slides and kidney damage is assessed by measuring the number of glomerular nuclei and the glomerular tuft area on H&E stained slides, the area of glomerular matrix and glomerular tuft area on silver-stained slides, and the area of glomerular MAC-2 positive staining for macrophages and the glomerular tuft area. Results are expressed as the cell number per glomerulus, the cell number per glomerular tuft area, the matrix area of each glomerulus, the percentage of matrix, the area of macrophages per glomerulus, and the area of macrophages per glomerular area.

Efficacy of FcγRIA are measured as decreases in the glomerular tuft area, mean glomerular areas occupied by macrophages, and mean cell numbers per glomerulus, and by decreases in matrix area, compared to wild-type controls.

### EXAMPLE 19

### FcγRIA Decreases Disease Incidence and Progression in Mouse Collagen Induced Arthritis (CIA) Model

### A. Mouse Collagen Induced Arthritis (CIA) Model

The CIA model of arthritis is an appropriate and well-regarded model to evaluate therapeutic potential of drugs to treat human arthritis. Arthritis is a disease that is characterized by inflammation and/or inappropriate immune complex formation with the joints. The immune complexes are often composed of antibodies directed against type II collagen, an important hyaline cartilage matrix protein. Formation of immune complexes within the joint leads to the recruitment of immune cells to the joint space and the generation of inflammatory cytokines that lead to cartilage and bone destruction within the affected joint. Collagen induced arthritis in mice thus shares many biochemical, cellular, and structural similarities with rheumatoid arthritis in humans.

Eight to ten-week old male DBA/1J mice (25-30 g) were used for these studies. On day −21, animals were given an intra-dermal tail injection of 0.1 mL of 1 mg/ml chick Type II collagen formulated in Complete Freund's Adjuvant (prepared by Chondrex Inc., Redmond, Wash.). Three weeks later, on Day 0, mice were given the same injection except prepared in Incomplete Freund's Adjuvant. Animals began to show symptoms of arthritis following the second collagen injection, with most animals developing inflammation within 1 to 2 weeks. The extent of disease was evaluated in each paw by using a caliper to measure paw thickness, and by assigning a clinical score (0-3) to each paw (see description below for disease scoring).

### B. Monitoring Disease

Incidence of disease in this model was 95-100% with only a few (0-2) non-responders (determined after 6 weeks of observation). Animals are considered to have established disease only after marked, persistent paw swelling has developed. All animals were observed daily to assess the status of the disease in their paws, which was done by assigning a qualitative clinical score to each of the paws. Every day, each animal had its 4 paws scored according to its state of clinical disease. To determine the clinical score, the paw is thought of as having 3 zones, the toes, the paw itself (manus or pes), and the wrist or ankle joint. The extent and severity of the inflammation relative to these zones was noted including: observation of each toe for swelling; torn nails or redness of toes; notation of any evidence of edema or redness in any of the paws; notation of any loss of fine anatomic demarcation of tendons or bones; evaluation of the wrist or ankle for any edema or redness; and notation if the inflammation extends proximally up the leg. A paw score of 1, 2, or 3 was based first on the overall impression of severity, and second on how many zones are involved. The scale used for clinical scoring is shown below:

Clinical Score

0=Normal

0.5=One or more toes involved, but only the toes are inflamed

1=mild inflammation involving the paw (1 zone), and may include a toe or toes

2=moderate inflammation in the paw and may include some of the toes and/or the wrist/ankle (2 zones)

3=severe inflammation in the paw, wrist/ankle, and some or all of the toes (3 zones)

### C. Treatments

Established disease was defined as a qualitative score of paw inflammation ranking 1 or more. Once established disease was present, the date was recorded, designated as that animal's first day with “established disease,” and treatment started. Mice were treated with PBS, or one of the following doses of human FcγRIA (hFcγRIA; diluted in PBS to desired concentration) subcutaneously every other day for a total of 6 doses: 2 mg; 0.667 mg; 0.22 mg; or one of the following doses of hFcγRIA (diluted in PBS to desired concentration) subcutaneously every 4th day for a total of 3 doses: 2 mg; 0.667 mg.

Blood was collected at the end of the experimental period to monitor serum levels of anti-collagen antibodies, as well as serum immunoglobulin and cytokine levels. Animals were euthanized 48 hours following their last treatment. Blood was collected for serum, and all paws and selected tissues were collected into 10% NBF for histology. Serum was collected and frozen at −800 C for immunoglobulin and cytokine assays.

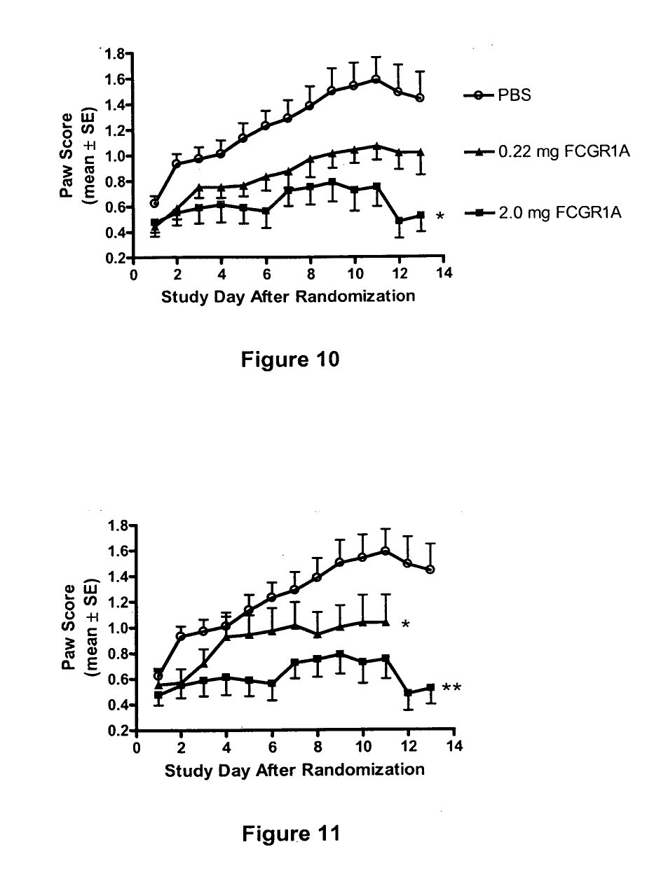

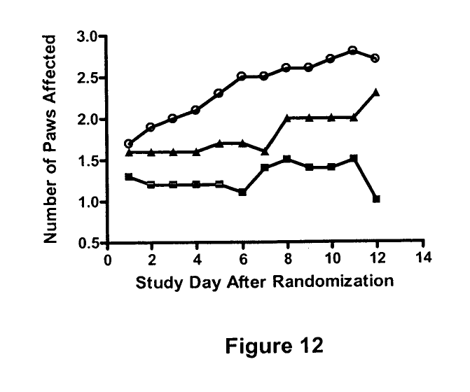

Mice injected with type II collagen and treated with vehicle developed paw swelling that was evident as higher disease scores (paw scores) with days after randomization (see FIG. 10, open circles). Treatment with FcγRIA every other day for 12 days produced a statistically significant, dose-dependent reduction in clinical scores (see FIG. 10, solid symbols). Treatment with the 0.22 mg dose produced a 50% reduction in disease progression, while the 2.0 mg dose reduced disease severity by 90%. Reduction in paw scores was also seen when FcγRIA was administered with an extended dose interval (see FIG. 11). Compared to treatment with vehicle alone (PBS), treatment with 2.0 mg of FcγRIA every fourth day for 9 days produced a 50% reduction in clinical scores, compared with the 90% reduction seen when FcγRIA was administered every other day (see FIG. 11). Mice treated with hFcγRIA also had a dose-dependent reduction in the number of affected paws (see FIG. 12).

In summary, these results indicate that in murine collagen-induced arthritis, administration of recombinant human FcγRIA can reduce disease incidence and progression. These data support the use of FcγRIA as a novel effective therapy for treatment of arthritis and other IgG- and immune complex-mediated diseases in humans.

### EXAMPLE 20

### FcγRIA Decreases Levels of IL-6 and Anti-type II Collagen Antibodies in Mouse Collagen Induced Arthritis (CIA) Model

In addition to monitoring disease development in the mouse CIA model by assessing the extent and severity of paw inflammation, mice used in the CIA study described above (see Example 19) were also assessed for levels of IL-6 and anti-type II collage antibodies, as summarized below.

### A. Methods

Quantitation of Serum Cytokines by Luminex Assay

The level of cytokines in mouse sera were quantitated using a Luminex cytokine assay kit from Upstate Biotechnology. Each plate was blocked with 0.2 mL of Assay Buffer for 10 min, the buffer was removed and the plate blotted. A 0.025 mL of each standard, control, blank, and test sample was added to the appropriate wells followed by a 0.025 mL sample of Serum Matrix. A 0.025 mL volume of Assay Buffer was added to each sample well followed by 0.025 mL of capture beads that were suspended by sonication. Each plate was sealed, covered in foil, and incubated on a shaker at 4° C. After 18-24 h, the well contents were removed by aspiration and the plate was blotted. Each plate was then washed 2-3 times with 0.2 mL of wash buffer, 0.025 mL of Detection Antibody Cocktail was added to each well and the plate was sealed, covered in foil, and incubated on a shaker at room temperature for 60 min. A 0.025 mL sample of Streptavidin-Phycoerythrin was added to each well, each plate was sealed, covered in foil, and incubated on a shaker at room temperature for 30 min. The contents of each well were removed by aspiration, each plate was blotted, and washed 2-3 times with 0.2 ml/well of wash buffer. A 0.1 ml sample of Sheath Buffer was added to each well and the absorbance of each sample was read on a Luminex instrument.

Quantitation of Anti-type II Collagen Antibodies

The level of anti-type II collagen antibodies in mouse sera were quantified using a Mouse IgG Anti-Type II Collagen Antibody Kit from Chondrex. Each plate was blocked with 0.1 mL of Blocking Buffer for 60 min at room temperature. The plates were washed three times with Wash Buffer and standards, samples, or blanks were added to the appropriate wells in a final volume of 0.1 mL. The plates were covered and incubated overnight at 4° C. The next day, each plate was washed six times with Wash Buffer and a 0.1 mL volume of secondary antibody was added to each well. The plates were then incubated at room temperature. After 2.0 h, each plate was washed and 0.1 mL of OPD solution was added to each well and incubated for 30 min at room temperature. The reactions were terminated by adding 0.05 mL of 2N sulfuric acid to each well and the absorbance of each well at 490 nm was determined.

### B. Results

Compared to non-arthritic mice that did not receive injections of type-II collagen, mice injected with type-II collagen had elevated serum levels of IL-6 at the time of sacrifice on day 15. Levels of IL-6 were below the level of detection in normal mice and increased to 320 pg/mL in mice that developed collagen-induced arthritis and were treated with vehicle alone. Treatment with soluble human FcγRIA (2.0 mg given every other day for two weeks) reduced the serum levels of IL-6 by 70% to 95 pg/mL on day 15.

In addition to reducing the levels of IL-6, treatment with soluble human FcγRIA also reduced the levels of anti-type II collagen antibodies in the sera of arthritic mice. Administration of 2.0 mg of FcγRIA every other day produced a 40-50% reduction in the amount of anti-type II collagen antibodies, relative to the levels observed in arthritic mice treated with vehicle alone, on day 15 at the time of sacrifice.

Although the foregoing invention has been described in detail for purposes of clarity of understanding, it will be appreciated that certain modifications may be practiced within the scope of the appended claims. All publications and patent documents cited herein are hereby incorporated by reference in their entirety for all purposes to the same extent as if each were so individually denoted.

What is claimed is:

1. A method of reducing IgG-mediated inflammation in a subject, the method comprising:
administering to a subject with IgG-mediated inflammation an effective amount of a soluble FcγRIA polypeptide, wherein the soluble FcγRIA polypeptide (i) comprises an amino acid sequence having at least 90% sequence identity with amino acid residues 16-282 of SEQ ID NO:2, and (ii) is capable of specifically binding the Fc region of IgG.

2. The method of claim 1, wherein the IgG-mediated inflammation is immune complex-mediated.

3. The method of claim 1, wherein the soluble FcγRIA polypeptide comprises an amino acid sequence having at least 95% sequence identity with amino acid residues 16-282 of SEQ ID NO:2.

4. The method of claim 1, wherein the soluble FcγRIA polypeptide comprises amino acid residues 16-282 of SEQ ID NO:2.

5. The method of claim 1, wherein the soluble FcγRIA polypeptide comprises amino acid residues 16-292 of SEQ ID NO:2.

6. The method of claim 1, wherein the soluble FcγRIA polypeptide consists of amino acid residues 16-X of SEQ ID NO:2, wherein X is an integer from 282 to 292, inclusive.

7. A method of treating an IgG-mediated inflammatory disease, the method comprising:
administering an effective amount of a soluble FcγRIA polypeptide to a subject having the IgG-mediated inflammatory disease, wherein the soluble FcγRIA polypeptide (i) comprises an amino acid sequence having at least 90% sequence identity amino acid residues 16-282 of SEQ ID NO:2, and (ii) is capable of specifically binding the Fc region of IgG.

8. The method of claim 7, wherein the soluble FcγRIA polypeptide comprises an amino acid sequence having at least 95% sequence identity with amino acid residues 16-282 of SEQ ID NO:2.

9. The method of claim 7, wherein the soluble FcγRIA polypeptide comprises amino acid residues 16-282 of SEQ ID NO:2.

10. The method of claim 7, wherein the soluble FcγRIA polypeptide comprises amino acid residues 16-292 of SEQ ID NO:2.

11. The method of claim 7, wherein the soluble FcγRIA polypeptide consists of amino acid residues 16-X of SEQ ID NO:2, wherein X is an integer from 282 to 292, inclusive.

12. The method of claim 7, wherein the IgG-mediated inflammatory disease is selected from the group consisting of rheumatoid arthritis (RA); systemic lupus erythematosus (SLE); mixed cryoglobulinemia; mixed connective tissue disease; a disease associated with an exonegous antigen; idiopathic thrombocytopenia purpura (ITP); Sjogren's syndrome; anti-phospholipid antibody syndrome; dermatomyositis; Guillain-Barre syndrome; and Goodpasture's syndrome.

13. The method of claim 7, wherein the IgG-mediated inflammatory disease is an immune complex-mediated disease.

14. The method of claim 13, wherein the immune-complex-mediated disease is selected from the group consisting of rheumatoid arthritis (RA); systemic lupus erythematosus (SLE); mixed cryoglobulinemia; mixed connective tissue disease; and a disease associated with an exonegous antigen.

15. The method of claim 14, wherein the disease associated with an exogenous antigen is hepatitis-B-associated polyarteritis nodosa.

16. The method of claim 7, wherein the IgG-mediated inflammatory disease is an autoimmune disease.

17. The method of claim 16, wherein the autoimmune disease is selected from the group consisting of rheumatoid arthritis (RA); systemic lupus erythematosus (SLE); mixed connective tissue disease; idiopathic thrombocytopenia purpura (ITP); Sjogren's syndrome; anti-phospholipid antibody syndrome; dermatomyositis; Guillain-Barre syndrome; and Goodpasture's syndrome.

18. A method of reducing IgG-mediated inflammation in a subject, the method comprising:
administering to a subject with IgG-mediated inflammation an effective amount of a soluble FcγRIA polypeptide, wherein the soluble FcγRIA polypeptide is a polypeptide produced by the method comprising
(a) culturing a cell into which has been introduced an expression vector comprising the following operably linked elements: (i) a transcription promoter; (ii) a DNA segment encoding a soluble polypeptide comprising an amino acid sequence having at least 90% sequence identity with amino acid residues 16-282 of SEQ ID NO:2, wherein the encoded polypeptide is capable of specifically binding the Fc region of IgG; and (iii) a transcription terminator; wherein the cell expresses the polypeptide encoded by the DNA segment; and
(b) recovering the expressed polypeptide.

19. The method of claim 18, wherein the IgG-mediated inflammation is immune complex-mediated.

20. The method of claim 18, wherein the encoded polypeptide comprises an amino acid sequence having at least 95% sequence identity with amino acid residues 16-282 of SEQ ID NO:2.

21. The method of claim 18, wherein the encoded polypeptide comprises amino acid residues 16-282 of SEQ ID NO:2.

22. The method of claim 18, wherein the encoded polypeptide comprises amino acid residues 16-292 of SEQ ID NO:2.

23. The method of claim 18, wherein the encoded polypeptide consists of amino acid residues 16-X of SEQ ID NO:2, wherein X is an integer from 282 to 292, inclusive.

24. A method of treating an IgG-mediated inflammatory disease, the method comprising:
administering an effective amount of a soluble FcγRIA polypeptide to a subject having the IgG-mediated inflammatory disease, wherein the soluble FcγRIA polypeptide is a polypeptide produced by the method comprising
(a) culturing a cell into which has been introduced an expression vector comprising the following operably linked elements: (i) a transcription promoter; (ii) a DNA segment encoding a soluble polypeptide comprising an amino acid sequence having at least 90% sequence identity with amino acid residues 16-282 of SEQ ID NO:2, wherein the encoded polypeptide is capable of specifically binding the Fc region of IgG; and (iii) a transcription terminator; wherein the cell expresses the polypeptide encoded by the DNA segment; and
(b) recovering the expressed polypeptide.

25. The method of claim 24, wherein the encoded polypeptide comprises an amino acid sequence having at least 95% sequence identity with amino acid residues 16-282 of SEQ ID NO:2.

26. The method of claim 24, wherein the encoded polypeptide comprises amino acid residues 16-282 of SEQ ID NO:2.

27. The method of claim 24, wherein the encoded polypeptide comprises amino acid residues 16-292 of SEQ ID NO:2.

28. The method of claim 24, wherein the encoded polypeptide consists of amino acid residues 16-X of SEQ ID NO:2, wherein X is an integer from 282 to 292, inclusive.

29. The method of claim 24, wherein the IgG-mediated inflammatory disease is selected from the group consisting of rheumatoid arthritis (RA); systemic lupus erythematosus (SLE); mixed cryoglobulinemia; mixed connective tissue disease; a disease associated with an exonegous antigen; idiopathic thrombocytopenia purpura (ITP); Sjogren's syndrome; anti-phospholipid antibody syndrome; dermatomyositis; Guillain-Barre syndrome; and Goodpasture's syndrome.

30. The method of claim 24, wherein the IgG-mediated inflammatory disease is an immune complex-mediated disease.

31. The method of claim 30, wherein the immune-complex-mediated disease is selected from the group consisting of rheumatoid arthritis (RA); systemic lupus erythematosus (SLE); mixed cryoglobulinemia; mixed connective tissue disease; and a disease associated with an exonegous antigen.

32. The method of claim 31, wherein the disease associated with an exogenous antigen is hepatitis-B-associated polyarteritis nodosa.

33. The method of claim 24, wherein the IgG-mediated inflammatory disease is an autoimmune disease.

34. The method of claim 33, wherein the autoimmune disease is selected from the group consisting of rheumatoid arthritis (RA); systemic lupus erythematosus (SLE); mixed connective tissue disease; idiopathic thrombocytopenia purpura (ITP); Sjogren's syndrome; anti-phospholipid antibody syndrome; dermatomyositis; Guillain-Barre syndrome; and Goodpasture's syndrome.

35. A method of treating an IgG-mediated inflammatory disease, the method comprising:
administering an effective amount of a soluble FcγRIA polypeptide to a subject having the IgG-mediated inflammatory disease, wherein the soluble FcγRIA polypeptide (i) comprises an amino acid sequence having at least 90% sequence identity amino acid residues 1-267 of SEQ ID NO:47, and (ii) is capable of specifically binding the Fc region of IgG.

36. The method of claim 35, wherein the soluble FcγRIA polypeptide comprises an amino acid sequence having at least 95% sequence identity with amino acid residues 1-267 of SEQ ID NO:47.

37. The method of claim 35, wherein the soluble FcγRIA polypeptide comprises amino acid residues 1-267 of SEQ ID NO:47.

38. The method of claim 35, wherein the soluble FcγRIA polypeptide comprises amino acid residues 1-277 of SEQ ID NO:47.

39. The method of claim 35, wherein the soluble FcγRIA polypeptide consists of amino acid residues 1-X of SEQ ID NO:47, wherein X is an integer from 267 to 277, inclusive.

40. The method of claim 35, wherein the IgG-mediated inflammatory disease is selected from the group consisting of rheumatoid arthritis (RA); systemic lupus erythematosus (SLE); mixed cryoglobulinemia; mixed connective tissue disease; a disease associated with an exonegous antigen; idiopathic thrombocytopenia purpura (ITP); Sjogren's syndrome; anti-phospholipid antibody syndrome; dermatomyositis; Guillain-Barre syndrome; and Goodpasture's syndrome.

41. The method of claim 35, wherein the IgG-mediated inflammatory disease is an immune complex-mediated disease.

42. The method of claim 41, wherein the immune-complex-mediated disease is selected from the group consisting of rheumatoid arthritis (RA); systemic lupus erythematosus (SLE); mixed cryoglobulinemia; mixed connective tissue disease; and a disease associated with an exonegous antigen.

43. The method of claim 42, wherein the disease associated with an exogenous antigen is hepatitis-B-associated polyarteritis nodosa.

44. The method of claim 35, wherein the IgG-mediated inflammatory disease is an autoimmune disease.

45. The method of claim 44, wherein the autoimmune disease is selected from the group consisting of rheumatoid arthritis (RA); systemic lupus erythematosus (SLE); mixed connective tissue disease; idiopathic thrombocytopenia purpura (ITP); Sjogren's syndrome; anti-phospholipid antibody syndrome; dermatomyositis; Guillain-Barre syndrome; and Goodpasture's syndrome.
